(19) United States
(12) Patent Application Publication
(10) Pub. No.: US 2012/0016390 A1
(43) Pub. Date: Jan. 19, 2012

(54) BIOADHESIVE COMPOUNDS AND METHODS OF SYNTHESIS AND USE

(75) Inventors: Bruce P. Lee, Madison, WI (US); John L. Murphy, Verona, WI (US); Laura Vollenweider, Lodi, WI (US); Jeffrey L. Dalsin, Verona, WI (US); Arinne N. Lyman, Fitchburg, WI (US)

(73) Assignee: KNC NER ACQUISITION SUB, INC., Wilmington, DE (US)

(21) Appl. No.: 13/184,234

(22) Filed: Jul. 15, 2011

Related U.S. Application Data
(60) Provisional application No. 61/365,049, filed on Jul. 16, 2010.

(51) Int. Cl. A61B 17/03; C08G 63/08; C08L 67/04; C08G 63/06

(52) U.S. Cl. 606/151; 528/332; 528/354; 525/450

(57) ABSTRACT

The invention describes new synthetic medical adhesives and antifouling coatings which exploit the key components of natural marine mussel adhesive proteins.

### CROSS REFERENCE TO RELATED APPLICATIONS

This application claims priority to U.S. Provisional application 61/365,049, filed Jul. 16, 2010, entitled “Bioadhesive Compounds and Methods of Synthesis and Use,” the contents of which is incorporated herein by reference for all purposes.

### REFERENCE TO FEDERAL FUNDING

The project was funded in part by NIH (1R43AR056519-01A1, 1R43DK083199-01, 2 R44DK083199-02, 1R43DK080547-01, 1R43DE017827-01, and 2R44DE017827-02), and NSF (IIP-0912221) grants. NMR characterization was performed at NMRFAM, which is supported by NIH(P41RR02301, P41GM66326, P41GM66326, P41RR02301, RR02781, RR08438) and NSF (DMB-8415048, OIA-9977486, BIR-9214394) grants. The government has certain rights in the invention.

### FIELD OF THE INVENTION

The invention relates generally to new synthetic medical adhesives which exploit the key components of natural marine mussel adhesive proteins. The method exploits a biological strategy to modify surfaces that exhibit adhesive properties useful in a diverse array of medical applications. Specifically, the invention describes the use of peptides that mimic natural adhesive proteins in their composition and adhesive properties. These adhesive moieties are coupled to a polymer chain, and provide adhesive and crosslinking (cohesive properties) to the synthetic polymer.

### BACKGROUND OF THE INVENTION

Mussel adhesive proteins (MAPs) are remarkable underwater adhesive materials secreted by certain marine organisms which form tenacious bonds to the substrates upon which they reside. During the process of attachment to a substrate, MAPs are secreted as adhesive fluid precursors that undergo a crosslinking or hardening reaction which leads to the formation of a solid adhesive plaque. One of the unique features of MAPs is the presence of L-3-4-dihydroxyphenylalanine (DOPA), an unusual amino acid which is believed to be responsible for adhesion to substrates through several mechanisms that are not yet fully understood. The observation that mussels adhere to a variety of surfaces in nature (metal, metal oxide, polymer) led to a hypothesis that DOPA-containing peptides can be employed as the key components of synthetic medical adhesives or coatings.

For example, bacterial attachment and biofilm formation are serious problems associated with the use of urinary stents and catheters as they often lead to chronic infections that cannot be resolved without removing the device. Although numerous strategies have been employed to prevent these events including the alteration of device surface properties, the application of anti-attachment and antibacterial coatings, host dietary and urinary modification, and the use of therapeutic antibiotics, no one approach has yet proved completely effective. This is largely due to three important factors, namely various bacterial attachment and antimicrobial resistance strategies, surface masking by host urinary and bacterial constituents, and biofilm formation. While the urinary tract has multiple anti-infective strategies for dealing with invading microorganisms, the presence of a foreign stent or catheter provides a novel, non-host surface to which they can attach and form a biofilm. This is supported by studies highlighting the ability of normally non-uropathogenic microorganisms to readily cause device-associated urinary tract infections. Ultimately, for a device to be clinically successful it must not only resist bacterial attachment but that of urinary constituents as well. Such a device would better allow the host immune system to respond to invading organisms and eradicate them from the urinary tract.

For example, bacterial attachment and subsequent infection and encrustation of uropathogenic E. coli (UPEC) cystitis is a serious condition associated with biofouling. Infections with E. coli comprise over half of all urinary tract device-associated infections, making it the most prevalent pathogen in such episodes.

Additionally, in the medical arena, few adhesives exist which provide both robust adhesion in a wet environment and suitable mechanical properties to be used as a tissue adhesive or sealant. For example, fibrin-based tissue sealants (e.g. Tisseel V H, Baxter Healthcare) provide a good mechanical match for natural tissue, but possess poor tissue-adhesion characteristics. Conversely, cyanoacrylate adhesives (e.g. Dermabond, ETHICON, Inc.) produce strong adhesive bonds with surfaces, but tend to be stiff and brittle in regard to mechanical properties and tend to release formaldehyde as they degrade.

Therefore, a need exists for materials that overcome one or more of the current disadvantages.

### BRIEF SUMMARY OF THE INVENTION

The present invention surprisingly provides multi-armed phenyl derivatives (PDs) comprising, for example, multihydroxy(dihydroxy)phenyl derivatives (DHPDs) having the general formula (I):

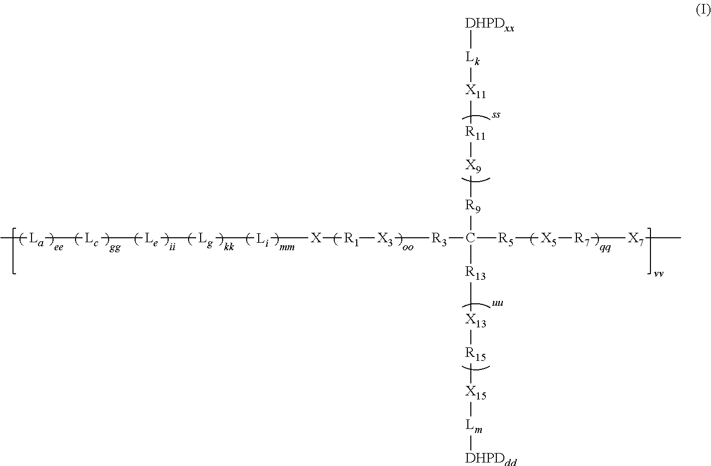

wherein

each Lₐ, L_{c}, Lₑ, L_{g} and Lᵢ, independently, is a linker;

each Lₖ and Lₘ, independently, is a linker or a suitable linking group selected from amine, amide, ether, ester, urea, carbonate or urethane linking groups;

each X, X₃, X₅, X₇, X₉, X₁₁, X₁₃ and X₁₅, independently, is an oxygen atom or NR;

R, if present, is H or a branched or unbranched C1-10 alkyl group;

each R₁, R₃, R₅, R₇, R₉, R₁₁, R₁₃ and R₁₅, independently, is a branched or unbranched C1-C15 alkyl group;

each DHPDₓₓ and DHPD_{dd}, independently, is a multihydroxy phenyl derivative residue;

ee is a value from 1 to about 80;

gg is a value from 0 to about 80:

ii is a value from 0 to about 80;

kk is a value from 0 to about 80;

mm is a value from 0 to about 80;

oo is a value from 1 to about 120;

qq is a value from 1 to about 120;

ss is a value from 1 to about 120;

uu is a value from 1 to about 120; and

vv is a value from 1 to about 80.

In one aspect, the compound of formula (I) Lₐ is a residue of succinic acid; Lₑ is a residue of a polycaprolactone or polylactic acid (thus forming an ester bond between terminal ends of the succinic acid and the hydroxyloxygen of the ring opened lactone); Lₑ is a residue of diethylene glycol (thus forming an ester bond between the ester portion of the lactone and one terminal hydroxyl group of the glycol); L_{g} is a residue of a polycaprolactone or a polylactic acid (therefore forming an ester linkage between a second terminal end of a hydroxyl group of the glycol and the ring opened caprolactone); L, is a residue of succinic acid or anhydride; X, X₇, X₁₁ and X₁₅ are each O or NH; R₁, R₇, R₁₁ and R₁₅ are each —CH₂CH₂— (thus forming a an amide or ester with the terminal end of an amine or hydroxyl terminated polyethylene glycol polyether); X₃, X₅, X₉ and X₁₃ are each 0; R₃, R₅, R₉ and R₁₃ are each —CH₂—; Lₖ and Lₘ form an amide linkage between the terminal end of the DHPD and the respective X; and DHPDₓₓ and DHPD_{dd} are 3,4-dihydroxyhydrocinnamic acid (DOHA) residues.

In another aspect, Lₐ is a residue of glycine; L_{c} is a residue of a polycaprolactone or a polylactic acid; Lₑ is a residue of diethylene glycol; L_{g} is a residue of a polycaprolactone or a polylactic acid; Lᵢ is a residue of glycine; X, X₇, X₁₁ and X₁₅ are each O or NH; R₁, R₇, R₁₁ and R₁₅ are each —CH₂CH₂—; X₃, X₅, X₉ and X₁₃ are each 0; R₃, R₅, R₉ and R₁₃ are each —CH₂—; Lₖ and Lₘ form a carbamate; and DHPDₓₓ and DHPD_{dd} are residues from 3,4 dihydroxyphenylethylamine.

In yet another aspect, Lₐ is a residue of a poly(ethyleneglycol) bis(carboxymethyl)ether; L_{c}, Lₑ, L_{g}, and Lᵢ are absent; ee is a value from 1 to about 11; gg, ii, kk, and mm are each independently 0; X, X₇, X₁₁ and X₁₅ are each O or NH; R₁, R₇, R₁₁ and R₁₅ are each —CH₂CH₂—; X₃, X₅, X₉ and X₁₃ are each 0; R₃, R₅, R₉ and R₁₃ are each —CH₂—; Lₖ and Lₘ form an amide; and DHPDₓₓ and DHPD_{dd} are residues from 3,4-dihydroxyhydrocinnamic acid (DOHA).

In still another aspect, FIG. 1 provides compounds I(a) through I(g) that depict certain embodiments of the invention.

Compound I(a), for example, has a Wt % DH (DOHA) of about 3.58+/−0.33%, Wt % PCL of about 12%, MW of about 97,650 g/mol with a PD of about 2.78.

Compound I(b), for example, has a Wt % DH of about 2.92+/−0.34%, Wt % PCL of about 20.7, MW of about 65,570 g/mol with a PD of about 4.414. MW's and PD were determined by gel permeation chromatography.

In one embodiment, the reaction products of the syntheses described herein are included as compounds or compositions useful as adhesives or surface treatment/antifouling aids. It should be understood that the reaction product(s) of the synthetic reactions can be purified by methods known in the art, such as diafiltration, chromatography, recrystallization/precipitation and the like or can be used without further purification.

In still another aspect, blends of the compounds of the invention described herein, can be prepared with various polymers. Polymers suitable for blending with the compounds of the invention are selected to impart non-covalent interactions with the compound(s), such as hydrophobic-hydrophobic interactions or hydrogen bonding with an oxygen atom on PEG and a substrate surface. These interactions can increase the cohesive properties of the film to a substrate. If a biopolymer is used, it can introduce specific bioactivity to the film, (i.e. biocompatibility, cell binding, immunogenicity, etc.).

Generally, there are four classes of polymers useful as blending agents with the compounds of the invention. Class 1 includes: Hydrophobic polymers (polyesters, PPG) with terminal functional groups (—OH, COOH, etc.), linear PCL-diols (MW 600-2000), branched PCL-triols (MW 900), wherein PCL can be replaced with PLA, PGA, PLAGA, and other polyesters.

Class 2 includes amphiphilic block (di, tri, or multiblock) copolymers of PEG and polyester or PPG, tri-block copolymers of PCL-PEG-PCL (PCL MW=500-3000, PEG MW=500-3000), tri-block copolymers of PLA-PEG-PLA (PCL MW=500-3000, PEG MW=500-3000). In other embodiments, PCL and PLA can be replaced with PGA, PLGA, and other polyesters. Pluronic polymers (triblock, diblock of various MW) and other PEG, PPG block copolymers are also suitable.

Class 3 includes hydrophilic polymers with multiple functional groups (—OH, —NH2, —COOH) along the polymeric backbone. These include, for example, PVA (MW 10,000-100,000), poly acrylates and poly methacrylates, and polyethylene imines.

Class 4 includes biopolymers such as polysaccharides, hyaluronic acid, chitosan, cellulose, or proteins, etc. which contain functional groups.

Abbreviations: PCL=polycaprolactone, PLA=polylactic acid, PGA=Polyglycolic acid, PLGA=a random copolymer of lactic and glycolic acid, PPG=polypropyl glycol, and PVA=polyvinyl alcohol.

It should be understood that the compounds of the invention can be coated multiple times to form bi, tri, etc. layers. The layers can be of the compounds of the invention per se, or of blends of a compound(s) and polymer, or combinations of a compound layer and a blend layer, etc.

Consequently, constructs can also include such layering of the compounds per se, blends thereof, and/or combinations of layers of a compound(s) per se and a blend or blends.

These adhesives of the invention described throughout the specification can be utilized for wound closure and materials of this type are often referred to as tissue sealants or surgical adhesives.

The compounds of the invention can be applied to a suitable substrate surface as a film or coating. Application of the compound(s) to the surface inhibits or reduces the growth of biofilm (bacteria) on the surface relative to an untreated substrate surface. In other embodiments, the compounds of the invention can be employed as an adhesive.

Exemplary applications include, but are not limited to fixation of synthetic (resorbable and non-resorbable) and biological membranes and meshes for hernia repair, void-eliminating adhesive for reduction of post-surgical seroma formation in general and cosmetic surgeries, fixation of synthetic (resorbable and non-resorbable) and biological membranes and meshes for tendon and ligament repair, sealing incisions after ophthalmic surgery, sealing of venous catheter access sites, bacterial barrier for percutaneous devices, as a contraceptive device, a bacterial barrier and/or drug depot for oral surgeries (e.g. tooth extraction, tonsillectomy, cleft palate, etc.), for articular cartilage repair, for antifouling or anti-bacterial adhesion.

In some embodiments, bioadhesives of the present invention are employed in constructs with polymer blends as described, for example in International Patent Application No. PCT/US2010/023382, International Filing Date: 5 Feb. 2010 entitled: “BIOADHESIVE CONSTRUCTS WITH POLYMER BLENDS”, incorporated by reference herein in its entirety.

While multiple embodiments are disclosed, still other embodiments of the present invention will become apparent to those skilled in the art from the following detailed description. As will be apparent, the invention is capable of modifications in various obvious aspects, all without departing from the spirit and scope of the present invention. Accordingly, the detailed descriptions are to be regarded as illustrative in nature and not restrictive.

### BRIEF DESCRIPTION OF THE DRAWINGS

FIG. 1 provides compounds I(a) through I(g) as embodiments of the present invention.

FIG. 2 depicts peak stress required to separate two pieces of adhered collagen sheets in burst strength test. Mode of failure: DNF=Did not fail; A=Adhesive failure; C=Cohesive failure. α=statistically different from Dermabond; β=statistically different from Medhesive-061. (p=0.05).

FIG. 3 provides a graphical representation of peak stress required to separate two pieces of adhered collagen sheets in lap shear mode. Mode of failure: A=Adhesive failure; C=Cohesive failure. α=statistically different from Dermabond; β=statistically different from Medhesive-061. (p=0.05).

FIG. 4 shows peak stress required to separate two pieces of adhered collagen sheets in lap shear mode. Mode of failure: A=Adhesive failure; C=Cohesive failure. β32 statistically different from Medhesive-054 (LN003135). (p=0.05).

FIG. 5 provides peak stress required to separate two pieces of adhered collagen sheets in lap shear mode. Mode of failure: A=Adhesive failure; C=Cohesive failure. β32 statistically different from Medhesive-054. (p=0.05).

FIG. 6 shows peak stress required to separate two pieces of adhered collagen sheets in lap shear mode. Mode of failure: A=Adhesive failure; C=Cohesive failure. α=statistically different from Dermabond; β=statistically different from Medhesive-061. (p=0.05).

FIG. 7 depicts the peak stress required to separate two pieces of adhered collagen sheets in lap shear mode. Mode of failure: A=Adhesive failure; C=Cohesive failure. α=statistically different from Dermabond. (p=0.05).

FIG. 8 provides a graphical representation of the work of adhesion required to separate two pieces of adhered collagen sheets in lap shear mode. Mode of failure: A=Adhesive failure; C=Cohesive failure. α=statistically different from Dermabond. (p=0.05).

FIG. 9 shows strain at failure for two pieces of adhered collagen sheets separated via lap shear mode. Mode of failure: A=Adhesive failure; C=Cohesive failure. α=statistically different from Dermabond. (p=0.05).

FIG. 10 depicts peak stress required to separate two pieces of adhered collagen sheets in lap shear mode. Mode of failure: A=Adhesive failure; C=Cohesive failure. α=statistically different from Medhesive-054. (p=0.05).

FIG. 11 shows bacterial adhesion on coated PVC.

FIG. 12 shows bacterial adhesion on coated Acetal.

FIG. 13 is a depiction of schematics of A) lap shear and B) burst strength test setups.

FIG. 14 shows the pressure required to burst through the adhesive joint sealed with adhesive-coated bovine pericardium. Dashed lines represent reported abdominal pressure range. Solid line represents statistical equivalence (p>0.05).

FIG. 15 shows the lap shear adhesive strength required to separate the adhesive joint formed using adhesive-coated bovine pericardium. Solid line represents statistical equivalence (p>0.05).

FIG. 16 provides schematics of A) control construct with 100% area coverage, B) a patterned construct with 8 circular uncoated areas (diameter=1.6 mm), and C), a patterned construct with 2 circular uncoated areas (diameter=0.5 mm).

FIG. 17 provides the lap shear adhesive strength required to separate the adhesive joint formed using adhesive-coated mesh applied to bovine pericardium.

FIG. 18 provides a mesh coated with adhesive pads.

FIG. 19 provides schematics of A) construct with 100% area coverage, B) a patterned construct with 2 circular uncoated areas with larger diameter, and C), a patterned construct with 8 circular uncoated areas with smaller diameter.

FIG. 20 shows degradation rate of Medhesive-096 and 054 at 55° C. in PBS.

FIG. 21 represents a schematic of multi-layer adhesive films.

FIG. 22 represents another schematic of multi-layer adhesive films.

FIG. 23 provides compound Medhesive-132, an embodiment of the present invention.

FIG. 24 provides compound Medhesive-136, an embodiment of the present invention.

FIG. 25 provides compound Medhesive-137, an embodiment of the present invention.

FIG. 26 provides compound Medhesive-138, an embodiment of the present invention.

FIG. 27 provides compound Medhesive-139, an embodiment of the present invention.

FIG. 28 provides compound Medhesive-140, an embodiment of the present invention.

FIG. 29 provides compound Medhesive-141, an embodiment of the present invention.

FIG. 30 provides compound Medhesive-142, an embodiment of the present invention.

FIG. 31 shows the percent dry mass remaining for 240 g/m² Medhesive-132 coated on PE mesh incubated in PBS (pH 7.4) at 37° C.

FIG. 32 provides a photograph of adhesive coated on a PTFE (Motif) mesh.

FIG. 33 shows peak lap shear stress of adhesive coated on PTFE mesh. Adhesive coating density is 150 g/m².

FIG. 34 shows peak lap shear stress of adhesive coated on PTFE mesh at a coating density of 240 g/m²

FIG. 35 shows peak lap shear stress of adhesive coated on human dermis at a coating density of 150 g/m². Adhesive joint area is 3 cm×1 cm.

FIG. 36 shows peak lap shear stress of adhesive coated on bovine pericardium.

FIG. 37 shows photographs of ovine rotator cuff primary repair augmented with A) sutured Biotape and B) Medhesive-137-coated Biotape construct.

FIG. 38 shows that formulations Medhesive-054 and -096 may be cytotoxic. L-929 cell viability is shown with un-crosslinked and crosslinked Medhesive-054 and Mehesive-096 before and after crosslinking with NaIO₃.

FIG. 39 shows that in dose response elution testing, sodium iodate (NaIO₃) may be cytotoxic at quantities greater than 1-10 mM. L-929 cell viability is shown to be a function of NaIO₃ dose.

FIG. 40 depicts polymers functionalized with a methoxy group at the meta-position (compound 2) compared to a dihydroxy catechol (compound 1). Chemical structures with (1) a catechol with —OH groups at 3 and 4 positions, and (2) 3-methoxy, 4-hydroxy-phenyl groups are shown.

FIG. 41 shows a cytotoxicity assay using the agarose overlay method (ISO 10993-5). Agarose overlay cytoxicity assays are performed on the negative HDPE and positive (Latex) controls. The arrow points to a zone of cell death.

FIG. 42 depicts a modification in chemical architecture, wherein a hydrolysable ester linkage is inserted between the hydrophilic PEG and adhesive molecule, DHP.

FIG. 43 shows a method to embed an oxidant using a multi-layer approach.

FIG. 44 shows that when a controlled amount of oxidant is delivered to the adhesive film and reduced to its benign form prior to contact with the abdominal wall, the adhesive retains adhesive performance and reproducibility using both PP and PE meshes.

FIG. 45 shows a segment of adhesive-coated mesh secured to the dorsal surface of the intact peritoneum in an “underlay” position on each side of an incision.

FIG. 46 shows the position of non-absorbable attachment sutures. Adhesives are coated onto segments of light-weight polyester mesh according to the pattern shown such that both ends of the segment of mesh are coated with adhesive, and the middle portion remains uncoated and accessible to tissue ingrowth. Fixation of certain coated meshes may be by adhesive alone, the adhesive fixation of other coated meshes may be reinforced on four sides with non-absorbable sutures (black dots).

FIG. 47 shows a photograph of a 4 cm×8 cm adhesive film (A) coated onto a 6 cm×8 cm segment of Biotape (B).

FIG. 48 shows close-up images of the gap formation during tensile testing of the sutured tendons loaded at A) 0 N (6 cm between grips), B) 50 N and C) 100 N, and D) sutured tendon augmented with adhesive-coated bovine pericardium loaded at 100 N. Solid arrows indicate gap formation for tendons repaired with suture alone.

FIG. 49 shows maximum lap shear strength using bovine pericardium as a test substrate. Both Tisseel and Dermabond were applied in situ to fix 2 pieces of bovine pericardium together following manufacturer's protocols. Mean lap shear strengths for AC1 and AC2 were significantly greater than for both Tisseel and Dermabond, and significantly less than for Dermabond (p<0.05).

FIG. 50 shows tensile failure testing of one tendon repaired with suture alone (A), and representative curves for each type of repaired tendon (B). (1) Toe region, (2) dashed line indicating the linear stiffness of the repaired tendon, (3) arrows indicating the first parallel suture being pulled off, which was considered to be failure of the repair (failure load), (4) energy to failure as calculated by the area under the curve up to the failure load, and (5) peak load where 3-loop suture begins to fail.

FIG. 51 shows that varying oxidant concentration (for n>12) demonstrate no statistical difference in average peak stress observed over the concentrations tested.

FIG. 52 shows the implantation sites of 2″×3″ polyester meshes meshes coated with adhesive in a pattern (75% coverage), and throughout the entirety of the mesh (100% coverage).

FIG. 53 shows patterns of tissue ingrowth.

FIG. 54 shows significant tissue ingrowth in the regions not coated with adhesive where the tissue remained attached to the mesh. A photograph of patterned adhesive-coated mesh viewed underneath a layer of peritoneum after 14-day implantation is shown. Arrows point to regions not coated with adhesive, with adhesive construct conforming to the tissue.

FIG. 55 shows significant tissue ingrowth (arrows) in the regions not coated with adhesive where the tissue remained attached to the mesh. A photograph of patterned adhesive-coated mesh after it was subjected to mechanical testing is shown. Arrows point to areas not coated with adhesive demonstrating significant amount of tissue ingrowth with tissue still remain attached to the mesh. A dashed line indicates where mesh has torn during tensile testing.

FIG. 56 shows patterns of 5-mm circles not coated with Medhesive-141 and Medhesive-142 for rapid tissue ingrowth. Dimensions of an adhesive-coated mesh with uncoated regions (10-mm diameter circles) are shown.

FIG. 57 shows patterns of 5-mm circles not coated with Medhesive-141 and Medhesive-142 for rapid tissue ingrowth on a PE mesh.

FIG. 58 shows an adhesive-coated patterned mesh inserted in between peritoneum and abdominal muscle wall. The adhesive was activated with the moisture in the tissue, which dissolved and released the oxidant during hydration.

FIG. 59 shows a photograph of in-situ activated adhesive-coated mesh with the construct conforming to the shape of the tissue.

FIG. 60 shows histology at day 14 after implantation to evaluated tissue response and initial tissue ingrowth.

FIG. 61 shows histology at day 14 after implantation to evaluated tissue response and initial tissue ingrowth.

### DETAILED DESCRIPTION

In the specification and in the claims, the terms “including” and “comprising” are open-ended terms and should be interpreted to mean “including, but not limited to . . . .” These terms encompass the more restrictive terms “consisting essentially of” and “consisting of.”

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, “characterized by” and “having” can be used interchangeably.

Unless defined otherwise, all technical and scientific terms used herein have the same meanings as commonly understood by one of ordinary skill in the art to which this invention belongs. All publications and patents specifically mentioned herein are incorporated by reference in their entirety for all purposes including describing and disclosing the chemicals, instruments, statistical analyses and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

“Alkyl,” by itself or as part of another substituent, refers to a saturated or unsaturated, branched, straight-chain or cyclic monovalent hydrocarbon radical derived by the removal of one hydrogen atom from a single carbon atom of a parent alkane, alkene or alkyne. Typical alkyl groups include, but are not limited to, methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl(allyl), cycloprop-1-en-1-yl; cycloprop-2-en-1-yl, prop-1-yn-1-yl, prop-2-yn-1-yl, etc.; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, etc.; and the like.

The term alkoxy (“OR”) includes groups where R is an hydrogen or an alkane chain linked to at least one oxygen.

The term “alkyl” is specifically intended to include groups having any degree or level of saturation, i.e., groups having exclusively single carbon-carbon bonds, groups having one or more double carbon-carbon bonds, groups having one or more triple carbon-carbon bonds and groups having mixtures of single, double and triple carbon-carbon bonds. Where a specific level of saturation is intended, the expressions “alkanyl,” “alkenyl,” and “alkynyl” are used. Preferably, an alkyl group comprises from 1 to 15 carbon atoms (C₁-C₁₅ alkyl), more preferably from 1 to 10 carbon atoms (C₁-C₁₀ alkyl) and even more preferably from 1 to 6 carbon atoms (C₁-C₆ alkyl or lower alkyl).

“Alkanyl,” by itself or as part of another substituent, refers to a saturated branched, straight-chain or cyclic alkyl radical derived by the removal of one hydrogen atom from a single carbon atom of a parent alkane. Typical alkanyl groups include, but are not limited to, methanyl; ethanyl; propanyls such as propan-1-yl, propan-2-yl(isopropyl), cyclopropan-1-yl, etc.; butanyls such as butan-1-yl, butan-2-yl(sec-butyl), 2-methyl-propan-1-yl(isobutyl), 2-methyl-propan-2-yl(t-butyl), cyclobutan-1-yl, etc.; and the like.

“Alkenyl,” by itself or as part of another substituent, refers to an unsaturated branched, straight-chain or cyclic alkyl radical having at least one carbon-carbon double bond derived by the removal of one hydrogen atom from a single carbon atom of a parent alkene. The group may be in either the cis or trans conformation about the double bond(s). Typical alkenyl groups include, but are not limited to, ethenyl; propenyls such as prop-1-en-1-yl, prop-1-en-2-yl, prop-2-en-1-yl(allyl), prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like.

“Alkyldiyl” by itself or as part of another substituent refers to a saturated or unsaturated, branched, straight-chain or cyclic divalent hydrocarbon group derived by the removal of one hydrogen atom from each of two different carbon atoms of a parent alkane, alkene or alkyne, or by the removal of two hydrogen atoms from a single carbon atom of a parent alkane, alkene or alkyne. The two monovalent radical centers or each valency of the divalent radical center can form bonds with the same or different atoms. Typical alkyldiyl groups include, but are not limited to, methandiyl; ethyldiyls such as ethan-1,1-diyl, ethan-1,2-diyl, ethen-1,1-diyl, ethen-1,2-diyl; propyldiyls such as propan-1,1-diyl, propan-1,2-diyl, propan-2,2-diyl, propan-1,3-diyl, cyclopropan-1,1-diyl, cyclopropan-1,2-diyl, prop-1-en-1,1-diyl, prop-1-en-1,2-diyl, prop-2-en-1,2-diyl, prop-1-en-1,3-diyl, cycloprop-1-en-1,2-diyl, cycloprop-2-en-1,2-diyl, cycloprop-2-en-1,1-diyl, prop-1-yn-1,3-diyl, etc.; butyldiyls such as, butan-1,1-diyl, butan-1,2-diyl, butan-1,3-diyl, butan-1,4-diyl, butan-2,2-diyl, 2-methyl-propan-1,1-diyl, 2-methyl-propan-1,2-diyl, cyclobutan-1,1-diyl; cyclobutan-1,2-diyl, cyclobutan-1,3-diyl, but-1-en-1,1-diyl, but-1-en-1,2-diyl, but-1-en-1,3-diyl, but-1-en-1,4-diyl, 2-methyl-prop-1-en-1,1-diyl, 2-methanylidene-propan-1,1-diyl, buta-1,3-dien-1,1-diyl, buta-1,3-dien-1,2-diyl, buta-1,3-dien-1,3-diyl, buta-1,3-dien-1,4-diyl, cyclobut-1-en-1,2-diyl, cyclobut-1-en-1,3-diyl, cyclobut-2-en-1,2-diyl, cyclobuta-1,3-dien-1,2-diyl, cyclobuta-1,3-dien-1,3-diyl, but-1-yn-1,3-diyl, but-1-yn-1,4-diyl, buta-1,3-diyn-1,4-diyl, etc.; and the like. Where specific levels of saturation are intended, the nomenclature alkanyldiyl, alkenyldiyl and/or alkynyldiyl is used. Where it is specifically intended that the two valencies are on the same carbon atom, the nomenclature “alkylidene” is used. In preferred embodiments, the alkyldiyl group comprises from 1 to 6 carbon atoms (C1-C6 alkyldiyl). Also preferred are saturated acyclic alkanyldiyl groups in which the radical centers are at the terminal carbons, e.g., methandiyl(methano); ethan-1,2-diyl(ethano); propan-1,3-diyl(propano); butan-1,4-diyl (butano); and the like (also referred to as alkylenos, defined infra).

“Alkyleno,” by itself or as part of another substituent, refers to a straight-chain saturated or unsaturated alkyldiyl group having two terminal monovalent radical centers derived by the removal of one hydrogen atom from each of the two terminal carbon atoms of straight-chain parent alkane, alkene or alkyne. The locant of a double bond or triple bond, if present, in a particular alkyleno is indicated in square brackets. Typical alkyleno groups include, but are not limited to, methano; ethylenos such as ethano, etheno, ethyno; propylenos such as propano, prop[1]eno, propa[1,2]dieno, prop[1]yno, etc.; butylenos such as butano, but[1]eno, but[2]eno, buta[1,3]dieno, but[1]yno, but[2]yno, buta[1,3]diyno, etc.; and the like. Where specific levels of saturation are intended, the nomenclature alkano, alkeno and/or alkyno is used. In preferred embodiments, the alkyleno group is (C1-C6) or (C1-C3) alkyleno. Also preferred are straight-chain saturated alkano groups, e.g., methano, ethano, propano, butano, and the like.

“Alkylene” by itself or as part of another substituent refers to a straight-chain saturated or unsaturated alkyldiyl group having two terminal monovalent radical centers derived by the removal of one hydrogen atom from each of the two terminal carbon atoms of straight-chain parent alkane, alkene or alkyne. The locant of a double bond or triple bond, if present, in a particular alkylene is indicated in square brackets. Typical alkylene groups include, but are not limited to, methylene (methano); ethylenes such as ethano, etheno, ethyno; propylenes such as propano, prop[1]eno, propa[1,2]dieno, prop[1]yno, etc.; butylenes such as butano, but[1]eno, but[2]eno, buta[1,3]dieno, but[1]yno, but[2]yno, buta[1,3]diyno, etc.; and the like. Where specific levels of saturation are intended, the nomenclature alkano, alkeno and/or alkyno is used. In preferred embodiments, the alkylene group is (C1-C6) or (C1-C3) alkylene. Also preferred are straight-chain saturated alkano groups, e.g., methano, ethano, propano, butano, and the like.

“Substituted,” when used to modify a specified group or radical, means that one or more hydrogen atoms of the specified group or radical are each, independently of one another, replaced with the same or different substituent(s). Substituent groups useful for substituting saturated carbon atoms in the specified group or radical include, but are not limited to —R^{a}, halo, —O⁻, ═O, —OR^{b}, —SR^{b}, —S⁻, ═S, —NR^{c}R^{c}, ═NR^{b}, ═N—OR^{b}, trihalomethyl, —CF₃, —CN, —OCN, —SCN, —NO, —NO₂, ═N₂, —N₃, —S(O)₂R^{b}, —S(O)₂O⁻, —S(O)₂OR^{b}, —OS(O)₂R^{b}, —OS(O)₂O⁻, —OS(O)₂OR^{b}, —P(O)(O⁻)₂, —P(O)(OR^{b})(O⁻), —P(O)(OR^{b})(OR^{b}), —C(O)R^{b}, —C(S)R^{b}, —C(NR^{b})R^{b}, —C(O)O⁻, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)⁻., —OC(O)OR^{b}, —OC(S)OR^{b}, —NR^{b}C(O)R^{b}, —NR^{b}C(S)R^{b}, —NR^{b}C(O)⁻, —NR^{b}C(O)OR^{b}, —NR^{b}C(S)OR^{b}, —NR^{b}C(O)NR^{c}R^{c}, —NR^{b}C(NR^{b})R^{b} and —NR^{b}C(NR^{b})NR^{c}R^{c}, where R^{a} is selected from the group consisting of alkyl, cycloalkyl, heteroalkyl, cycloheteroalkyl, aryl, arylalkyl, heteroaryl and heteroarylalkyl; each R^{b} is independently hydrogen or R^{a}; and each R^{c} is independently R^{b} or alternatively, the two R^{c}s are taken together with the nitrogen atom to which they are bonded form a 5-, 6- or 7-membered cycloheteroalkyl which may optionally include from 1 to 4 of the same or different additional heteroatoms selected from the group consisting of O, N and S. As specific examples, —NR^{c}R^{c} is meant to include —NH₂, —NH-alkyl, N-pyrrolidinyl and N-morpholinyl.

Similarly, substituent groups useful for substituting unsaturated carbon atoms in the specified group or radical include, but are not limited to, —R^{a}, halo, —O⁻, —OR^{b}, —SR^{b}, —S⁻, —NR^{c}R^{c}, trihalomethyl, —CF₃, —CN, —OCN, —SCN, —NO, —NO₂, —N₃, —S(O)₂R^{b}, —S(O)₂O⁻, —S(O)₂OR^{b}, —OS(O)₂R^{b}, —OS(O)₂O⁻, —OS(O)₂OR^{b}, —P(O)(O)₂, —P(O)(OR^{b})(O⁻), —P(O)(OR^{b})(OR^{b}), —C(O)R^{b}, —C(S)R^{b}, —C(NR^{b})R^{b}, —C(O)O⁻, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)O⁻, —OC(O)OR^{b}, —OC(S)OR^{b}, —NR^{b}C(O)R^{b}, —NR^{b}C(S)R^{b}, —NR^{b}C(O)O⁻, —NR^{b}C(O)OR^{b}, —NR^{b}C(S)OR^{b}, —NR^{b}C(O)NR^{c}R^{c}, —NR^{b}C(NR^{b})R^{b} and —NR^{b}C(NR^{b})NR^{c}R^{c}, where R^{a}, R^{b} and R^{c} are as previously defined.

Substituent groups useful for substituting nitrogen atoms in heteroalkyl and cycloheteroalkyl groups include, but are not limited to, —R^{a}, —O⁻, —OR^{b}, —SR^{b}, —S⁻., —NR^{c}R^{c}, trihalomethyl, —CF₃, —CN, —NO, —NO₂, —S(O)₂R^{b}, —S(O)₂O⁻, —S(O)₂OR^{b}, —OS(O)₂R^{b}, —OS(O)₂O⁻, —OS(O)₂OR^{b}, —P(O)(O⁻)₂, —P(O)(OR^{b})(O⁻), —P(O)(OR^{b})(OR^{b}), —C(O)R^{b}, —C(S)R^{b}, —C(NR^{b})R^{b}, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)OR^{b}, —OC(S)OR^{b}, —NR^{b}C(O)R^{b}, —NR^{b}C(S)R^{b}, —NR^{b}C(O)OR^{b}, —NR^{b}C(S)OR^{b}, —NR^{b}C(O)NR^{c}R^{c}, —NR^{b}C(NR^{b})R^{b} and —NR^{b}C(NR^{b})NR^{c}R^{c}, where R^{a}, R^{b} and R^{c} are as previously defined.

Substituent groups from the above lists useful for substituting other specified groups or atoms will be apparent to those of skill in the art.

The substituents used to substitute a specified group can be further substituted, typically with one or more of the same or different groups selected from the various groups specified above.

The identifier “PA” refers to a poly(alkylene oxide) or substantially poly(alkylene oxide) and means predominantly or mostly alkyloxide or alkyl ether in composition. This definition contemplates the presence of heteroatoms e.g., N, O, S, P, etc. and of functional groups e.g., —COOH, —NH₂, —SH, or —OH as well as ethylenic or vinylic unsaturation. It is to be understood any such non-alkyleneoxide structures will only be present in such relative abundance as not to materially reduce, for example, the overall surfactant, non-toxicity, or immune response characteristics, as appropriate, of this polymer. It should also be understood that PAs can include terminal end groups such as PA-O—CH₂—CH₂—NH₂, e.g., PEG-O—CH₂—CH₂—NH₂ (as a common form of amine terminated PA). PA-O—CH₂—CH₂—CH₂—NH₂, e.g., PEG-O—CH₂—CH₂—CH₂—NH₂ is also available as well as PA-O—(CH₂—CH(CH₃)—O)ₓₓ—CH₂—CH(CH₃)—NH₂, where xx is 0 to about 3, e.g., PEG-O—(CH₂—CH(CH₃)—O)ₓₓ—CH₂—CH(CH₃)—NH₂ and a PA with an acid end-group typically has a structure of PA-O—CH₂—COOH, e.g., PEG-O—CH₂—COOH or PA-O—CH, —CH, —COOH, e.g., PEG-O—CH₂—CH₂—COOH. These can be considered “derivatives” of the PA. These are all contemplated as being within the scope of the invention and should not be considered limiting.

Suitable PAs (polyalkylene oxides) include polyethylene oxides (PEOs), polypropylene oxides (PPOs), polyethylene glycols (PEGs) and combinations thereof that are commercially available from SunBio Corporation, JenKem Technology USA, NOF America Corporation or Creative PEGWorks. It should be understood that, for example, polyethylene oxide can be produced by ring opening polymerization of ethylene oxide as is known in the art.

In one embodiment, the PA can be a block copolymer of a PEO and PPO or a PEG or a triblock copolymer of PEO/PPO/PEO.

Suitable MW ranges of the PA's are from about 300 to about 8,000 daltons, 400 to about 5,000 daltons or from about 450 to about 3,500 daltons.

It should be understood that the PA terminal end groups can be functionalized. Typically the end groups are OH, NH₂, COOH, or SH. However, these groups can be converted into a halide (Cl, Br, I), an activated leaving group, such as a tosylate or mesylate, an ester, an acyl halide, N-succinimidyl carbonate, 4-nitrophenyl carbonate, and chloroformate with the leaving group being N-hydroxy succinimide, 4-nitrophenol, and Cl, respectively, etc.

The notation of “L” refers to either a linker or a linking group. A “linker” refers to a moiety that has two points of attachment on either end of the moiety. For example, an alkyl dicarboxylic acid HOOC-alkyl-COOH (e.g., succinic acid) would “link” a terminal end group of a PA (such as a hydroxyl or an amine to form an ester or an amide respectively) with a reactive group of the DHPD (such as an NH₂, OH, or COOH). Suitable linkers include an acyclic hydrocarbon bridge (e.g., a saturated or unsaturated alkyleno such as methano, ethano, etheno, propano, prop[1]eno, butano, but[1]eno, but[2]eno, buta[1,3]dieno, and the like), a monocyclic or polycyclic hydrocarbon bridge (e.g., [1,2]benzeno, [2,3]naphthaleno, and the like), a monocyclic or polycyclic heteroaryl bridge (e.g., [3,4]furano[2,3]furano, pyridino, thiopheno, piperidino, piperazino, pyrazidino, pyrrolidino, and the like) or combinations of such bridges, dicarbonyl alkylenes, etc. Suitable dicarbonyl alkylenes include, C2 through C15 dicarbonyl alkylenes such as malonic acid, succinic acid, etc. Additionally, the anhydrides, acid halides and esters of such materials can be used to effect the linking when appropriate and can be considered “activated” dicarbonyl compounds.

Other suitable linkers include moieties that have two different functional groups that can react and link with an end group of a PA. These include groups such as amino acids (glycine, lysine, aspartic acid, etc.), PA's as described herein, poly(ethyleneglycol) bis(carboxymethyl)ethers, polyesters such as polylactides, lactones, polylactones such as polycaprolactone, lactams, polylactams such as polycaprolactam, polyglycolic acid (PGLA), moieties such as tyramine or dopamine and random or block copolymers of 2 or more types of polyesters.

Linkers further include compounds comprising the formula Y₄—R₁₇—C(═O)—Y₆, wherein Y₄ is OH, NHR, a halide, or an activated derivative of OH or NHR; R₁₇ is a branched or unbranched C1-C15 alkyl group; and Y₆ is NHR, a halide, or OR, wherein R is defined above. The term “activated derivative” refers to moieties that make the hydroxyl or amine more susceptible to nucleophilic displacement or for condensation to occur. For example, a hydroxyl group can be esterified by various reagents to provide a more active site for reaction to occur.

A linking group refers to the reaction product of the terminal end moieties of the PA and DHPD (the situation where “b” is 0; no linker present) condense to form an amide, ether, ester, urea, carbonate or urethane linkage depending on the reactive sites on the PA and DHPD. In other words, a direct bond is formed between the PA and DHPD portion of the molecule and no linker is present.

The term “residue” is used to mean that a portion of a first molecule reacts (e.g., condenses or is an addition product via a displacement reaction) with a portion of a second molecule to form, for example, a linking group, such an amide, ether, ester, urea, carbonate or urethane linkage depending on the reactive sites on the PA and DHPD. This can be referred to as “linkage”.

The denotation “DHPD” refers to a multihydroxy phenyl derivative, such as a dihydroxy phenyl derivative, for example, a 3,4 dihydroxy phenyl moiety. Suitable DHPD derivatives include the formula:

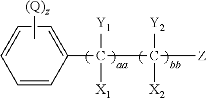

wherein Q is an OH;

“z” is 2 to 5;

each X₁, independently, is H, NH₂, OH, or COOH;

each Y₁, independently, is H, NH₂, OH, or COOH;

each X₂, independently, is H, NH₂, OH, or COOH;

each Y₂, independently, is H, NH₂, OH, or COOH;

Z is COOH, NH₂, OH or SH;

aa is a value of 0 to about 4;

bb is a value of 0 to about 4; and

optionally provided that when one of the combinations of X₁ and X₂, Y₁ and Y₂, X₁ and Y₂ or Y₁ and X₂ are absent, then a double bond is formed between the Cₐₐ and C_{bb}, further provided that aa and bb are each at least 1.

In one aspect, z is 3.

In particular, “z” is 2 and the hydroxyls are located at the 3 and 4 positions of the phenyl ring.

In one embodiment, each X₁, X₂, Y₁ and Y₂ are hydrogen atoms, aa is 1, bb is 1 and Z is either COOH or NH₂.

In another embodiment, X₁ and Y₂ are both hydrogen atoms, X₂ is a hydrogen atom, aa is 1, bb is 1, Y₂ is NH₂ and Z is COOH.

In still another embodiment, X₁ and Y₂ are both hydrogen atoms, aa is 1, bb is 0, and Z is COOH or NH₂.

In still another embodiment, aa is 0, bb is 0 and Z is COOH or NH₂.

In still yet another embodiment, z is 3, aa is 0, bb is 0 and Z is COOH or NH₂.

It should be understood that where aa is 0 or bb is 0, then X₁ and Y₁ or X₂ and Y₂, respectively, are not present.

It should be understood, that upon condensation of the DHPD molecule with the PA that a molecule of water, for example, is generated such that a bond is formed as described above (amide, ether, ester, urea, carbonate or urethane).

In particular, DHPD molecules include 3,4-dihydroxyphenethylamine (dopamine), 3,4-dihydroxy phenylalanine (DOPA), 3,4-dihydroxyhydrocinnamic acid, 3,4-dihydroxyphenyl ethanol, 3,4 dihydroxyphenylacetic acid, 3,4 dihydroxyphenylamine, 3,4-dihydroxybenzoic acid, etc.

The present invention surprisingly provides multi-armed, multihydroxy (dihydroxy)phenyl derivatives (DHPDs) having the general formula:

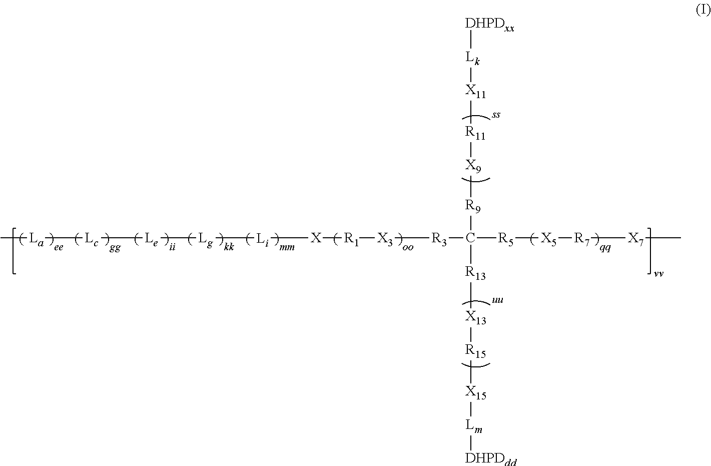

wherein

each Lₐ, L_{c}, Lₑ, L_{g} and Lᵢ, independently, is a linker;

each Lₖ and Lₘ, independently, is a linker or a suitable linking group selected from amine, amide, ether, ester, urea, carbonate or urethane linking groups;

each X, X₃, X₅, X₇, X₉, X₁₁, X₁₃ and X₁₅, independently, is an oxygen atom or NR;

R, if present, is H or a branched or unbranched C1-10 alkyl group;

each R₁, R₃, R₅, R₇, R₉, R₁₁, R₁₃ and R₁₅, independently, is a branched or unbranched C1-C15 alkyl group;

each DHPDₓₓ and DHPD_{dd}, independently, is a multihydroxy phenyl derivative residue;

ee is a value from 1 to about 80, in particular from 1 to about 50, more particularly, from 1 to about 20, and more particularly from 1 to about 10;

gg is a value from 0 to about 80, in particular from 1 to about 50, more particularly, from 1 to about 25, and more particularly from 1 to about 10;

ii is a value from 0 to about 80, in particular from 1 to about 50, more particularly, from 1 to about 25, and more particularly from 1 to about 15;

kk is a value from 0 to about 80, in particular from 1 to about 50, more particularly, from 1 to about 25, and more particularly from 1 to about 10;

mm is a value from 0 to about 80, in particular from 1 to about 50, more particularly, from 1 to about 20, and more particularly from 1 to about 10;

oo is a value from 1 to about 120, in particular from 1 to about 60, more particularly from 1 to about 30, and more particularly from 1 to about 10;

qq is a value from 1 to about 120, in particular from 1 to about 60, more particularly from 1 to about 30, and more particularly from 1 to about 10;

ss is a value from 1 to about 120, in particular from 1 to about 60, more particularly from 1 to about 30, and more particularly from 1 to about 10;

uu is a value from 1 to about 120, in particular from 1 to about 60, more particularly from 1 to about 30, and more particularly from 1 to about 10; and

vv is a value from 1 to about 80, in particular from 1 to about 50, more particularly, from 1 to about 20, and more particularly from 1 to about 10.

In one example, oo, qq, ss and uu are all about equal or equal.

For example, each Lₐ, L_{c}, Lₑ, L_{g} and Lᵢ, independently if present, is a linker selected from the residue of a C1-C15 alkyl anhydride or activated dicarbonyl moiety, a polyethylene glycol, a poly(ethyleneglycol) bis(carboxymethyl)ether, an amino acid, a C1-C15 alkyl lactone or lactam, a poly C1-C15 alkyl lactone or lactam, a polyester, a compound comprising the formula Y₄—R₁₇—C(═O)—Y₆, wherein Y₄ is OH, NHR, a halide, or an activated derivative of OH or NHR; R₁₇ is a branched or unbranched C1-C15 alkyl group; and Y₆ is NHR, a halide, or OR, wherein R is as described above, a residue of an C1-C15 alkylene diol, a C1-C15 alkylene diamine, a poly(alkylene oxide) polyether or derivative thereof or —O—CH₂CH₂—O—CH₂CH₂—O—.

In certain embodiments, Lₐ, when present, is a residue of a C1-C15, alkyl anhydride or activated dicarbonyl moiety, a poly(ethyleneglycol) bis(carboxymethyl)ether or an amino acid, wherein the activated dicarbonyl moiety is a residue of succinic acid or the amino acid is glycine.

In certain embodiments, L_{c}, when present, is a residue of a C1-C15 alkyl lactone or lactam, a poly C1-C15 alkyl lactone or lactam, a polyester, or a compound comprising the formula Y₄—R₁₇—C(═O)—Y₆, wherein Y₄ is OH, NHR, a halide, or an activated derivative of OH or NHR; R₁₇ is a branched or unbranched C1-C15 alkyl group; and Y₆ is NHR, a halide, or OR, wherein R is as described above. In particular, the polylactone is a polycaprolactone or the polyester is a polylactide (polylactic acid).

In certain embodiments, Lₑ when present, is a residue of an alkylene diol, such as a polyethylene glycol, an alkylene diamine or a poly(alkylene oxide) polyether or derivative thereof. In particular, Lₑ is a poly(alkylene oxide) or —O—CH₂CH₂—O—CH₂CH₂—O—.

In certain embodiments, L_{g}, when present, is a residue of a C1-C15 alkyl lactone or lactam, a poly C1-C15 alkyl lactone or lactam, or a compound comprising the formula Y₄—R₁₇—C(═O)—Y₆, wherein Y₄ is OH, NHR, a halide, or an activated derivative of OH or NHR; R₁₇ is a branched or unbranched C1-C15 alkyl group; and Y₆ is NHR, a halide, or OR, where R is described above. In particular, the polylactone is a polycaprolactone or the polyester is a polylactide (polylactic acid).

In certain embodiments, Lᵢ, when present, is a residue of a C1-C15 alkyl anhydride or activated dicarbonyl moiety, a poly(ethyleneglycol) bis(carboxymethyl)ether or an amino acid, wherein the activated dicarbonyl moiety is a residue of succinic acid or the amino acid is glycine.

In certain embodiments, X, X₇, X₁₁ and X₁₅ are each O or NH.

In certain embodiments, R₁, R₇, R₁₁ and R₁₅ are each —CH₂CH₂

In certain embodiments, X₃, X₅, X₉ and X₁₃ are each —O.

In certain embodiments, R₃, R₅, R₉ and R₁₃ are each —CH₂—.

In certain embodiments, Lₖ and Lₘ form/are an amide, ester or carbamate.

In certain embodiments, Lₐ as a residue of a poly(ethyleneglycol) bis(carboxymethyl)ether is not included as a linker.

It should be understood that a person having ordinary skill in the art would select appropriate combinations of linkers to provide an array of condensation products embodied and described herein.

In certain embodiments an oxidant is included with the bioadhesive film layer. The oxidant can be incorporated into the polymer film or it can be contacted to the film at a later time. A solution could be sprayed or brushed onto either the adhesive surface or the tissue substrate surface. Alternatively, the construct can be dipped or submerged in a solution of oxidant prior to contacting the tissue substrate. In any situation, the oxidant upon activation, can help promote crosslinking of the multihydroxy phenyl groups with each other and/or tissue. Suitable oxidants include periodates and the like.

The invention further provides crosslinked bioadhesive constructs or hydrogels derived from the compositions described herein. For example, two PD moieties from two separate polymer chains can be reacted to form a bond between the two PD moieties. Typically, this is an oxidative/radical initiated crosslinking reaction wherein oxidants/initiators such as NaIO₃, NaIO₄, Fe III salts, (FeCl₃), Mn III salts (MnCl₃), H₂O₂, oxygen, an inorganic base, an organic base or an enzymatic oxidase can be used. Typically, a ratio of oxidant/initiator to DHDP containing material is between about 0.1 to about 10.0 (on a molar basis) (oxidant:PD). In one particular embodiment, the ratio is between about 0.5 to about 5.0 and more particularly between about 1.0 to about 3.0. It has been found that periodate is very effective in the preparation of crosslinked hydrogels of the invention. Additionally, it is possible that oxidation “activates” the PD(s) which allow it to form interfacial crosslinking with appropriate surfaces with functional group (i.e., biological tissues with —NH2, —SH, etc.)

The compositions of the invention can be utilized by themselves or in combination with polymers to form a blend. Suitable polymers include, for example, polyesters, PPG, linear PCL-diols (MW 600-2000), branched PCL-triols (MW 900), wherein PCL can be replaced with PLA, PGA, PLGA, and other polyesters, amphiphilic block (di, tri, or multiblock) copolymers of PEG and polyester or PPG, tri-block copolymers of PCL-PEG-PCL (PCL MW=500-3000, PEG MW=500-3000), tri-block copolymers of PLA-PEG-PLA (PCL MW=500-3000, PEG MW=500-3000), wherein PCL and PLA can be replaced with PGA, PLGA, and other polyesters. Pluronic polymers (triblock, diblock of various MW) and other PEG, PPG block copolymers are also suitable. Hydrophilic polymers with multiple functional groups (—OH, —NH₂, —COOH) contained within the polymeric backbone such as PVA (MW 10,000-100,000), poly acrylates and poly methacrylates, polyvinylpyrrolidone, and polyethylene imines are also suitable. Biopolymers such as polysaccharides (e.g., dextran), hyaluronic acid, chitosan, gelatin, cellulose (e.g., carboxymethyl cellulose), proteins, etc. which contain functional groups can also be utilized.

Abbreviations: PCL=polycaprolactone, PLA=polylactic acid, PGA=Polyglycolic acid, PLGA=a random copolymer of lactic and glycolic acid, PPG=polypropyl glycol, and PVA=polyvinyl alcohol.

Typically, blends of the invention include from about 0 to about 99.9% percent (by weight) of polymer to composition(s) of the invention, more particularly from about 1 to about 50 and even more particularly from about 1 to about 30.

The compositions of the invention, either a blend or a compound of the invention per se, can be applied to suitable substrates using conventional techniques. Coating, dipping, spraying, spreading and solvent casting are possible approaches.

In one embodiment, adhesive compounds of the present invention provide a method of adhering a first surface to a second surface in a subject. In some embodiments, the first and second surfaces are tissue surfaces, for example, a natural tissue, a transplant tissue, or an engineered tissue. In further embodiments, at least one of the first and second surfaces is an artificial surface. In some embodiments, the artificial surface is an artificial tissue. In other embodiments, the artificial surface is a device or an instrument. In some embodiments, adhesive compounds of the present invention seal a defect between a first and second surface in a subject. In other embodiments, adhesive compounds of the present invention provide a barrier to, for example, microbial contamination, infection, chemical or drug exposure, inflammation, or metastasis. In further embodiments, adhesive compounds of the present invention stabilize the physical orientation of a first surface with respect to a second surface. In still further embodiments, adhesive compounds of the present invention reinforce the integrity of a first and second surface achieved by, for example, sutures, staples, mechanical fixators, or mesh. In some embodiments, adhesive compounds of the present invention provide control of bleeding. In other embodiments, adhesive compounds of the present invention provide delivery of drugs including, for example, drugs to control bleeding, treat infection or malignancy, or promote tissue regeneration.

The present invention surprisingly provides unique bioadhesive constructs that are suitable to repair or reinforce damaged tissue.

The present invention also surprisingly provides unique antifouling coatings/constructs that are suitable for application in, for example, urinary applications. The coatings could be used anywhere that a reduction in bacterial attachment is desired: dental unit waterlines, implantable orthopedic devices, cardiovascular devices, wound dressings, percutaneous devices, surgical instruments, marine applications, food preparation surfaces and utensils.

The constructs include a suitable support that can be formed from a natural material, such as collagen, pericardium, dermal tissues, small intestinal submucosa or man made materials such as polypropylene, polyethylene, polybutylene, polyesters, PTFE, PVC, polyurethanes and the like. The support can be a film, a membrane, a mesh, a non-woven and the like. The support need only help provide a surface for the bioadhesive to adhere. The support should also help facilitate physiological reformation of the tissue at the damaged site. Thus the constructs of the invention provide a site for remodeling via fibroblast migration, followed by subsequent native collagen deposition. For biodegradable support of either biological or synthetic origins, degradation of the support and the adhesive can result in the replacement of the bioadhesive construct by the natural tissues of the patient.

The constructs of the invention can include a compound of the invention or mixtures thereof or a blend of a polymer with one or more of the compounds of the invention. In one embodiment, the construct is a combination of a substrate, to which a blend is applied, followed by a layer(s) of one or more compounds of the invention.

In another embodiment, two or more layers can be applied to a substrate wherein the layering can be combinations of one or more blends or one or more compositions of the invention. The layering can alternate between a blend and a composition layer or can be a series of blends followed by a composition layer or vice versa.

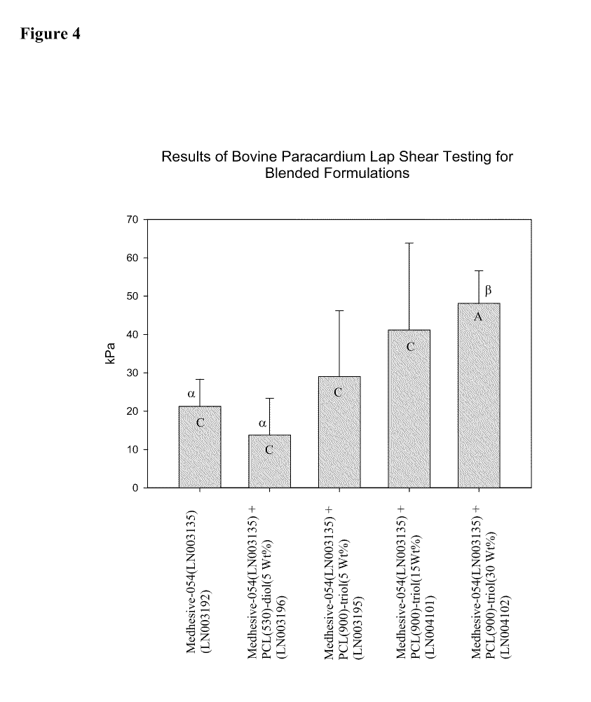

Not to be limited by theory, it is believe that to improve the overall adhesive strength of the present adhesives, two separate properties require consideration: 1) interfacial binding ability or “adhesion” to a substrate and 2) bulk mechanical properties or “cohesion”. It is possible that some polymers may generally fail cohesively, meaning that their adhesive properties are better than their cohesive properties. That is one basis why blending with a hydrophobic polymer increases the bulk cohesive properties. For example, an increase in the overall adhesive strength (FIG. 4) was found and we also a change in the mode of failure mode was also noted. For example, at the highest PCL content (30%), the blend failed adhesively, which supports the hypothesis that blending of PCL increases cohesive properties.

It has interestingly been found that use of a blend advantageously has improved adhesion to the substrate surface. For example, a blend of a hydrophobic polymer with a composition of the invention of Formula (I) has improved overall cohesive properties of Formula (I) and thus the overall strength of the adhesive joint. Subsequent application of a composition of Formula I to the blend layer then provides improved interfacial adhesion between the blend and provides for improved adhesive properties to the tissue to be adhered to as the hydrophobic polymer is not in the outermost layer.

Typically the loading density of the coating layer is from about 0.001 g/m² to about 400 g/m², more particularly from about 5 g/m² to about 150 g/m², and more particularly from about 10 g/m² to about 100 g/m². Thus, typically a coating has a thickness of from about 1 to about 200 nm. More typically for an adhesive, the thickness of the film is from about 1 to about 200 microns.

In some embodiments of the present invention, a bilayer comprises a non-reactive polymer (e.g., Medhesive-142) which comprises an oxidant, and a reactive adhesive layer (e.g., Medhesive-141). The reactive adhesive layer may have, for example, a density of 240 g/m², and the non-reactive layer comprising an oxidant may have, for example, a density of 120 g/m², for a total thin film density of 360 g/m².

The following paragraphs enumerated consecutively from 1 through 37 provide for various aspects of the present invention. In one embodiment, in a first paragraph (1), the present invention provides a compound comprising the formula (I)

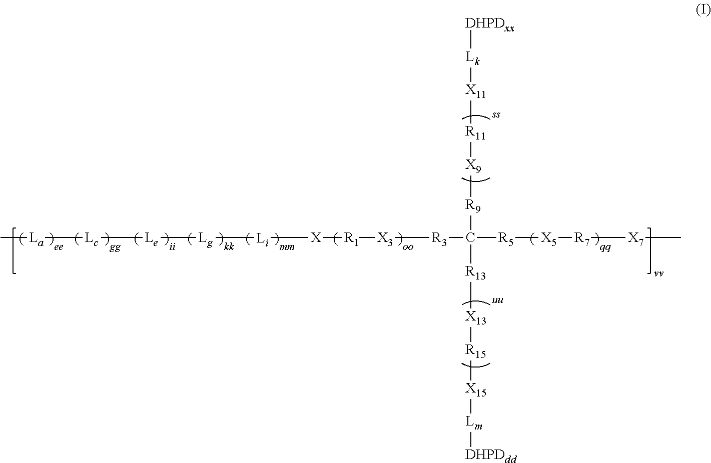

wherein

each Lₐ, L_{c}, Lₑ, L_{g} and Lᵢ, independently, is a linker;

each Lₖ and Lₘ, independently, is a linker or a suitable linking group selected from amine, amide, ether, ester, urea, carbonate or urethane linking groups;

each X, X₃, X₅, X₇, X₉, X₁₁, X₁₃ and X₁₅, independently, is an oxygen atom or NR;

R, if present, is H or a branched or unbranched C1-10 alkyl group;

each R₁, R₃, R₅, R₇, R₉, R₁₁, R₁₃ and R₁₅, independently, is a branched or unbranched C1-C15 alkyl group;

each DHPDₓₓ and DHPD_{dd}, independently, is a multihydroxy phenyl derivative residue;

ee is a value from 1 to about 80;

gg is a value from 0 to about 80:

ii is a value from 0 to about 80;

kk is a value from 0 to about 80;

mm is a value from 0 to about 80;

oo is a value from 1 to about 120;

qq is a value from 1 to about 120;

ss is a value from 1 to about 120;

uu is a value from 1 to about 120; and

vv is a value from 1 to about 80.

2. The compound of paragraph 1, wherein Lₐ is a residue of a C1-C15, alkyl anhydride or activated dicarbonyl moiety, a poly(ethyleneglycol) bis(carboxymethyl)ether, polyethylene glycol or an amino acid.

3. The compound of paragraph 2, wherein the dicarbonyl moiety is a residue of succinic acid or the amino acid is glycine.

4. The compound of any of paragraphs 1 through 3, wherein L_{c} is a residue of a C1-C15 alkyl lactone or lactam, a poly C1-C15 alkyl lactone or lactam, a polyester, or a compound comprising the formula Y₄—R₁₇—C(═O)—Y₆,

wherein Y₄ is OH, NHR, a halide, or an activated derivative of OH or NHR;

R₁₇ is a branched or unbranched C1-C15 alkyl group; and

Y₆ is NHR, a halide, or OR.

5. The compound of paragraph 4, wherein the polylactone is a polycaprolactone.

6. The compound of any of paragraphs 1 through 5, wherein Lₑ is a residue of an alkylene diol, an alkylene diamine or a poly(alkylene oxide) polyether or derivative thereof.

7. The compound of paragraph 6, wherein Lₑ is a poly(alkylene oxide) or —O—CH₂CH₂—O—CH₂CH₂—O—.

8. The compound of any of paragraphs 1 through 7, wherein L_{g} is a residue of a C1-C15 alkyl lactone or lactam, a poly C1-C15 alkyl lactone or lactam, or a compound comprising the formula Y₄—R₁₇—C(═O)—Y₆,

wherein Y₄ is OH, NHR, a halide, or an activated derivative of OH or NHR;

R₁₇ is a branched or unbranched C1-C15 alkyl group; and

Y₆ is NHR, a halide, or OR.

9. The compound of paragraph 8, wherein the polylactone is polycaprolactone.

10. The compound of any of paragraphs 1 through 9, wherein L, is a residue of a C1-C15 alkyl anhydride or activated dicarbonyl moiety, a poly(ethyleneglycol) bis(carboxymethyl)ether or an amino acid.

11. The compound of paragraph 10, wherein Lᵢ is a residue of succinic acid or glycine.

12. The compound of any of paragraphs 1 through 11, wherein X, X₇, X₁₁ and X₁₅ are each O or NH.

13. The compound of any of paragraphs 1 through 12, wherein R₁, R₇, R₁₁ and R₁₅ are each —CH₂CH₂—.

14. The compound of any of paragraphs 1 through 13, wherein X₃, X₅, X₉ and X₁₃ are each 0.

15. The compound of any of paragraphs 1 through 14, wherein R₃, R₅, R₉ and R₁₃ are each —CH₂—.

16. The compound of any of paragraphs 1 through 15, wherein Lₖ and Lₘ form an amide, ester or carbamate.

17. The compound of any of paragraphs 1 through 16, wherein each DHPD_{XX} and DHPD_{dd}, independently, is a residue of a formula comprising:

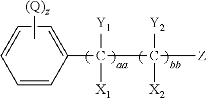

wherein Q is an OH;

“z” is 2 to 5;

each X₁, independently, is H, NH₂, OH, or COOH;

each Y₁, independently, is H, NH₂, OH, or COOH;

each X₂, independently, is H, NH₂, OH, or COOH;

each Y₂, independently, is H, NH₂, OH, or COOH;

Z is COOH, NH₂, OH or SH;

aa is a value of 0 to about 4;

bb is a value of 0 to about 4; and

optionally provided that when one of the combinations of X₁ and X₂, Y₁ and Y₂, X₁ and Y₂ or Y₁ and X₂ are absent, then a double bond is formed between the Cₐₐ and C_{bb}, further provided that aa and bb are each at least 1 to form the double bond when present.

18. The compound of any of paragraphs 1 through 17, wherein DHPD_{XX} and DHPD_{dd} residues are from 3,4-dihydroxy phenylalanine (DOPA), 3,4-dihydroxyhydrocinnamic acid (DOHA), 3,4-dihydroxyphenyl ethanol, 3,4 dihydroxyphenylacetic acid, 3,4 dihydroxyphenylamine, or 3,4-dihydroxybenzoic acid.

19. The compound of paragraph 1, wherein

Lₐ is a residue of succinic acid;

L_{c} is a residue of a polycaprolactone, a caprolactone, a polylactic acid, a polylactone or a lactic acid or lactone;

Lₑ is a residue of a polyethylene glycol, e.g., diethylene glycol;

L_{g} is a residue of a polycaprolactone, a caprolactone, a polylactic acid, a polylactone or a lactic acid or lactone;

Lᵢ is a residue of succinic anhydride;

X, X₇, X₁₁ and X₁₅ are each O or NH;

R₁, R₇, R₁₁ and R₁₅ are each —CH₂CH₂—;

X₃, X₅, X₉ and X₁₃ are each O;

R₃, R₅, R₉ and R₁₃ are each —CH₂—;

Lₖ and Lₘ, form an amide; and

DHPDₓ, and DHPD_{dd} are residues from 3,4-dihydroxyhydrocinnamic acid (DOHA).

20. The compound of paragraph 1, wherein

Lₐ is a residue of glycine;

L_{c} is a residue of a polycaprolactone;

Lₑ is a residue of a polyethylene glycol, e.g., diethylene glycol;

L_{g} is a residue of a polycaprolactone;

Lᵢ is a residue of glycine;

X, X₇, X₁₁ and X₁₅ are each O or NH;

R₁, R₇, R₁₁ and R₁₅ are each —CH₂CH₂—;

X₃, X₅, X₉ and X₁₃ are each O;

R₃, R₅, R₉ and R₁₃ are each —CH₂—;

Lₖ and Lₘ form a carbamate; and

DHPDₓₓ and DHPD_{dd} are residues from 3,4 dihydroxyphenylethylamine.

21. The compound of paragraph 1, wherein

Lₐ is a residue of a poly(ethyleneglycol) bis(carboxymethyl)ether;

L_{c}, Lₑ, L_{g}, and Lᵢ are absent;

ee is a value from 1 to about 11;

gg, ii, kk, and mm are each independently 0;

X, X₇, X₁₁ and X₁₅ are each O or NH;

R₁, R₇, R₁₁ and R₁₅ are each —CH₂CH₂—;

X₃, X₅, X₉ and X₁₃ are each O;

R₃, R₅, R₉ and R₁₃ are each —CH₂—;

Lₖ and Lₘ form an amide; and

DHPDₓₓ and DHPD_{dd} are residues from 3,4-dihydroxyhydrocinnamic acid (DOHA).

22. A bioadhesive construct, comprising:

a support suitable for tissue repair or reconstruction; and

a coating comprising a multihydroxyphenyl (DHPD) functionalized polymer (DHPp) of any of paragraphs 1 through 21.

23. The bioadhesive construct of paragraph 22, further comprising an oxidant.

24. The bioadhesive construct of either of paragraphs 22 or 23, wherein the oxidant is formulated with the coating.

25. The bioadhesive construct of either of paragraphs 22 or 23, wherein the oxidant is applied to the coating.

26. The bioadhesive construct of any of paragraphs 22 through 25, wherein the support is a film, a mesh, a membrane, a nonwoven or a prosthetic.

27. A blend of a polymer and a compound of any of paragraphs 1 through 21.

28. The blend of paragraph 27, wherein the polymer is present in a range of about 1 to about 50 percent by weight.

29. The blend of paragraph 28, wherein the polymer is present in a range of about 1 to about 30 percent by weight.

30. A bioadhesive construct comprising:

a support suitable for tissue repair or reconstruction; and

a coating comprising any of the blends of paragraphs 27 through 29.

31. The bioadhesive construct of paragraph 30, further comprising an oxidant.

32. The bioadhesive construct of either of paragraphs 30 or 31, wherein the oxidant is formulated with the coating.

33. The bioadhesive construct of either of paragraphs 30 or 31, wherein the oxidant is applied to the coating.

34. The bioadhesive construct of any of paragraphs 30 through 33, wherein the support is a film, a mesh, a membrane, a nonwoven or a prosthetic.

35. A bioadhesive construct comprising:

a support suitable for tissue repair or reconstruction;

a first coating comprising a multihydroxyphenyl (DHPD) functionalized polymer (DHPp) of any of paragraphs 1 through 21 and a polymer; and

a second coating coated onto the first coating, wherein the second coating comprises a multihydroxyphenyl (DHPD) functionalized polymer (DHPp) of any of paragraphs 1 through 21.

36. A bioadhesive construct comprising:

a support suitable for tissue repair or reconstruction;

a first coating comprising a first multihydroxyphenyl (DHPD) functionalized polymer (DHPp) of any of paragraphs 1 through 21 and a first polymer; and

a second coating coated onto the first coating, wherein the second coating comprises a second multihydroxyphenyl (DHPD) functionalized polymer (DHPp) of any of paragraphs 1 through 21 and a second polymer, wherein the first and second polymer may be the same or different and wherein the first and second DHPp can be the same or different.

37. A bioadhesive construct comprising:

a support suitable for tissue repair or reconstruction;

a first coating comprising a first multihydroxyphenyl (DHPD) functionalized polymer (DHPp) of any of paragraphs 1 through 21; and

a second coating coated onto the first coating, wherein the second coating comprises a second multihydroxyphenyl (DHPD) functionalized polymer (DHPp) of any of paragraphs 1 through 21, wherein the first and second DHPp can be the same or different.

The present invention surprisingly provides multi-armed phenyl derivatives (PDs) comprising, for example, multi-methoxy phenyl derivatives. The following paragraphs enumerated consecutively from 1 through 34 provide for various aspects of the present invention. In one embodiment, in a first paragraph (1), the present invention provides a compound comprising the formula (I)

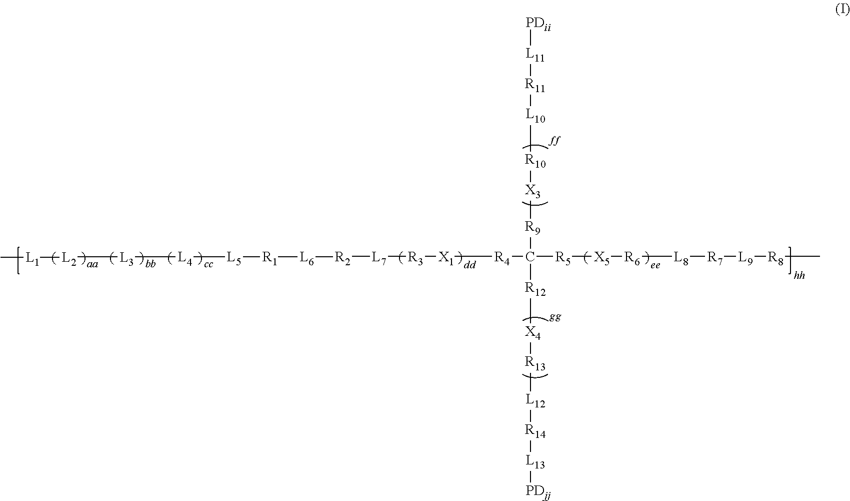

Wherein
each L₂, L₃ and L₄ independently, is a linker;
each L₁, L₅, L₆, L₇, L₈, L₉, L₁₀, L₁₁ L₁₂ and L₁₃, independently, is a linker or a suitable linking group selected from amine, amide, ether, ester, urea carbonate or urethane linking groups;
each X₁, X₂, X₃ and X₄ independently, is an oxygen atom or NR;

R, if present, is H or a branched or unbranched C1-C10 alkyl group;
each R₁, R₂, R₃, R₄, R₅, R₆, R₇, R₈, R₉, R₁₀, R₁₁, R₁₂, R₁₃ and R₁₄ independently, is a branched or unbranched C1-C15 alkyl group;
each PDᵢᵢ and PDⱼⱼ, independently, is a phenyl derivative residue;
aa is a value from 0 to about 80;

bb is a value from 0 to about 80;

cc is a value from 0 to about 80;

dd is a value from 1 to about 120;

ee is a value from 1 to about 120;

ff is a value from 1 to about 120;

gg is a value from 1 to about 120; and

hh is a value from 1 to about 80.

2. The compound of paragraph 1, wherein L₂ is a residue of a C1-C15 alkyl lactone or lactam, a poly C1-C15 alkyl lactone or lactam, a polyester, or a compound comprising the formula Y₄—R₁₇—C(═O)—Y₆, wherein Y₄ is OH, NHR, a halide, or an activated derivative of OH or NHR; R₁₇ is a branched or unbranched C1-C15 alkyl group; and Y₆ is NHR, a halide, or OR.

3. The compound of paragraph 2, wherein the polylactone is a polycaprolactone.

4. The compound of any of paragraphs 1 through 3, wherein L₃ is a residue of an alkylene diol, an alkylene diamine or a poly(alkylene oxide) polyether or derivative thereof.

5. The compound of paragraph 4, wherein L₃ is a poly(alkylene oxide) or —O—CH₂CH₂—O—CH₂CH₂—O—.

6. The compound of any of paragraphs 1 through 5, wherein L₂ or L₄ is a residue of a C1-C15 alkyl lactone or lactam, a poly C1-C15 alkyl lactone or lactam, or a compound comprising the formula Y₄—R₁₇—C(═O)—Y₆, wherein Y₄ is OH, NHR, a halide, or an activated derivative of OH or NHR; R₁₇ is a branched or unbranched C1-C15 alkyl group; and Yₒ is NHR, a halide, or OR.

7. The compound of paragraph 6, wherein the polylactone is polycaprolactone.

8. The compound of any of paragraphs 1 through 7, wherein X₁, X₂, X₃ and X₄ are each O or NH.

9. The compound of any of paragraphs 1 through 8, wherein R₃, R₆, R₁₀ and R₁₃ are each —CH₂CH₂—.

10. The compound of any of paragraphs 1 through 9, wherein X₁, X₂, X₃ and X₄ are each O.

11. The compound of any of paragraphs 1 through 10, wherein R₄, R₅, R₉ and R₁₂ are each —CH₂—.

12. The compound of any of paragraphs 1 through 11, wherein R₁, R₂, R₇, R₈, R₁₁ and R₁₄ are a branched or unbranched alkane.

13. The compound of paragraph 16, wherein R₁, R₂, R₇, R₈, R₁₁ and R₁₄ are —CH₂—CH₂— or CH₂—CH₂—CH₂—.

14. The compound of any of paragraphs 1 through 13, wherein L₁, L₅, L₆, L₇, L₈, L₉, L₁₀, L₁₁, L₁₂, and L₁₃ form an amide, ester or carbamate.

15. The compound of any of paragraphs 1 through 18, wherein each PDₓₓ and PD_{dd}, independently, is a residue of a formula comprising:

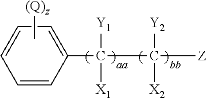

wherein Q is an OH or OCH3;

“z” is 1 to 5;

Each X₁, independently, is H, NH₂, OH, or COOH;

Each Y₁, independently, is H, NH₂, OH, or COOH;

Each X₂, independently, is H, NH₂, OH, or COOH;

Each Y₂, independently, is H, NH₂, OH, or COOH;

Z is COOH, NH₂, OH or SH;

aa is a value of 0 to about 4;

bb is a value of 0 to about 4; and

optionally provided that when one of the combinations of X₁ and X₂, Y₁ and Y₂, X₁ and Y₂ or

Y₁ and X₂ are absent, then a double bond is formed between the Cₐₐ and C_{bb}, further provided that aa and bb are each at least 1 to form the double bond when present.

16. The compound of any of paragraphs 1 through 19, wherein PDₓₓ and PD_{dd} residues are selected from the group consisting of 3,4-dihydroxyphenylalanine (DOPA), 3,4-dihydroxyphenethylamine (dopamine), 3,4-dihydroxyhydrocinnamic acid (DOHA), 3,4-dihydroxyphenyl ethanol, 3,4-dihydroxyphenylacetic acid, 3,4-dihydroxyphenylamine, 3,4-dihydroxybenzoic acid, 3-(3,4-dimethoxyphenyl)propionic acid, 3,4-dimethoxyphenylalanine, 2-(3,4-dimethoxyphenyl)ethanol, 3,4-dimethoxyphenethylamine, 3,4-dimethoxybenzylamine, 3,4-dimethoxybenzyl alcohol, 3,4-dimethoxyphenylacetic acid, 3-(3,4-dimethoxyphenyl)-2-hydroxypropanoic acid, 3,4-dimethoxybenzoic acid, 3,4-dimethoxyaniline, 3,4-dimethoxyphenol, 3-(4-Hydroxy-3-methoxyphenyl)propionic acid, homovanillyl alcohol, 3-methoxytyramine, 3-methoxy-L-tyrosine, homovanillic acid, 4-hydroxy-3-methoxybenzylamine, vanillyl alcohol, vanillic acid, 5-amino-2-methoxyphenol, 2-methoxyhydroquinone, 3-hydroxy-4-methoxyphenethylamine, 3-hydroxy-4-methoxyphenylacetic acid, 3-hydroxy-4-methoxyphenylacetic acid, 3-hydroxy-4-methoxybenzylamine, 3-hydroxy-4-methoxybenzyl alcohol, isovanillic acid.

17. The compound of paragraph 1, wherein

L₂ is a residue of a polycaprolactone, a caprolactone, a polylactic acid, a polylactone or a lactic acid or lactone;

L₃ is a residue of polyethylene glycol;

L₄ is a residue of a polycaprolactone, a caprolactone, a polylactic acid, a polylactone or a lactic acid or lactone;

X₁, X₂, X₃ and X₄ are each O or NH;

R₁, R₃, R₆, R₈, R₁₀, and R₁₃ are each —CH₂CH₂—;

R₄, R₅, R₉ and R₁₂ are each —CH₂—;

R₂, R₇, R₁₁ and R₁₄ are each —(CH₂)ₙ—, wherein n is 3;

L₁, L₅, L₇, L₈, L₁₀, L₁₂ form an ester;

L₆, L₉, L₁₁, and L₁₃ form an amide; and

PDₓₓ and PD_{dd} are residues selected from the group consisting of 3,4-dihydroxyhydrocinnamic acid (DOHA), hydroferulic acid (HFA), or 3,4-dimethoxyhydrocinnamic acid (3,4-DMHCA).

18. The compound of paragraph 1, wherein

L₂ is a residue of a polycaprolactone, a caprolactone, a polylactic acid, a polylactone or a lactic acid or lactone;

L₃ is a residue of polyethylene glycol;

L₄ is a residue of a polycaprolactone, a caprolactone, a polylactic acid, a polylactone or a lactic acid or lactone;

X₁, X₂, X₃ and X₄ are each O or NH;

R₃, R₆, R₁₀, and R₁₃ are each —CH₂CH₂—;

R₁, R₈, R₄, R₅, R₉ and R₁₂ are each —CH₂—;

R₂, R₇, R₁₁ and R₁₄ are each —(CH₂)ₙ—, wherein n is 2 or 3;

L₁, L₅, L₇, L₈, L₁₀, L₁₂ form an ester;

L₆, L₉, L₁₁, and L₁₃ form an amide; and

PDₓₓ and PD_{dd} are residues selected from the group consisting of 3,4-dihydroxyphenylethylamine, 3-methoxytyramine.

19. A bioadhesive construct, comprising:

a support suitable for tissue repair or reconstruction; and

a coating comprising a phenyl derivative (PD) functionalized polymer (PDp) of any of paragraphs 1 through 18.

20. The bioadhesive construct of paragraph 19, further comprising an oxidant.

21. The bioadhesive construct of either of paragraphs 19 or 20, wherein the oxidant is formulated with the coating.

22. The bioadhesive construct of either of paragraphs 19 or 20, wherein the oxidant is applied to the coating.

23. The bioadhesive construct of any of paragraphs 19 through 22, wherein the support is a film, mesh, a membrane, a nonwoven or a prosthetic.

24. A blend of a polymer and a compound of any of paragraphs 1 through 18.

25. The blend of paragraph 24, wherein the polymer is present in a range of about 1 to about 50 percent by weight.

26. The blend of paragraph 25, wherein the polymer is present in a range of about 30 percent by weight.

27. A bioadhesive construct comprising:

a support suitable for tissue repair or reconstruction; and

a coating comprising any of the blends of paragraphs 24 through 26.

28. The bioadhesive construct of paragraph 27, further comprising an oxidant.

29. The bioadhesive construct of either of paragraphs 27 or 28, wherein the oxidant is formulated with the coating.

30. The bioadhesive construct of either of paragraphs 27 or 28, wherein the oxidant is applied to the coating.

31. The bioadhesive construct of any of paragraphs 27 through 30, wherein the support is a film, a mesh, a membrane, a nonwoven or a prosthetic.

32. A bioadhesive construct comprising:

a support suitable for tissue repair or reconstruction;

a first coating comprising a phenyl derivative (PD) functionalized polymer (PDp) of any of paragraphs 1 through 18 and a polymer; and
a second coating coated onto the first coating, wherein the second coating comprises a phenyl derivative (PD) functionalized polymer (PDp) of any of paragraphs 1 through 18.

33. A bioadhesive construct comprising:

a support suitable for tissue repair or reconstruction;

a first coating comprising a first phenyl derivative (PD) functionalized polymer (PDp) of any of paragraphs 1 through 18 and a first polymer; and

a second coating coated onto the first coating, wherein the second coating comprises a second phenyl derivative (PD) functionalized polymer (PDp) of any of paragraphs 1 through 18 and a second polymer, wherein the first and second polymer may be the same or different and wherein the first and second PDp can be the same or different.

34. A bioadhesive construct comprising:

a support suitable for tissue repair or reconstruction;

a first coating comprising a first phenyl derivative (PD) functionalized polymer (PDp) of

any of paragraphs 1 through 18; and

a second coating coated onto the first coating, wherein the second coating comprises a second phenyl derivative (PD) functionalized polymer (PDp) of any of paragraphs 1 through 18, wherein the first and second PDp can be the same or different.

In some embodiments of the present invention, PDs comprise one, two or more hydroxy phenyl derivatives. In other embodiments, PDs comprise one, two or more methoxy phenyl derivatives. In still further embodiments, PDs comprise at least one hydroxyl and at least one methoxy phenyl derivatives.

In some embodiments, the polymer may be configured to desired biodegradability by eliminating one or more ester linking groups binding PEG to PD or PCL.

The invention will be further described with reference to the following non-limiting Examples. It will be apparent to those skilled in the art that many changes can be made in the embodiments described without departing from the scope of the present invention. Thus the scope of the present invention should not be limited to the embodiments described in this application, but only by embodiments described by the language of the claims and the equivalents of those embodiments. Unless otherwise indicated, all percentages are by weight.

Experimental Examples

### Example 1

### Synthesis of Surphys-029

10 g of 4-arm PEG-NH₂ (10,000 MW; 1 mmol), 600 mg of poly(ethyleneglycol) bis(carboxymethyl)ether (PEG-bCME, Mn ˜600, 1 mol), and 456 mg of 3,4-dihydroxyhydrocinnamic acid (DOHA, 2.5 mmol) was dissolved with 40 ml chloroform and 20 ml DMF in a round bottom flask equipped with an addition funnel. 676 mg of HOBt (5 mmol), 1.9 g of HBTU (5 mmol), and 560 μL of triethylamine (4 mmol) in 30 mL of DMF were added dropwise to the round bottom flask over a period of 90 minutes. The mixture was stirred at room temperature for 2 hours and added to 600 mL of diethyl ether. The precipitate was collected via vacuum filtration and dried. The crude product was further purified through dialysis (15,000 MWCO) in deionized H₂O (acidified to pH 3.5) for 24 hrs. After lyophilization, 6.3 g of Surphys-029 was obtained. ¹H NMR (400 MHz, D₂O): δ6.85-6.67 (m, 3H, C₆H₃(OH)₂—), 4.09 (s, 2H, PEG—CH₂—O—C(O)—NH—), 3.75-3.28 (m, PEG), 2.8 (t, 2H, C₆H₃(OH)₂—CH₂—CH₂—C(O)—NH—), 2.51 (t, 2H, C₆H₃(OH)₂—CH₂—CH₂—C(O)—NH—). UV-vis spectroscopy: 0.21±0.019 μmole DH/mg polymer (3.5±0.32 wt % DH). GPC: Mw=140,000, Mn=43,000, PD=3.3.

### Example 2

### Synthesis of PCL1.25 k-diSA

10 g of polycaprolactone-diol (PCL-diol, MW=1,250, 8 mmol), 8 g of succinic anhydride (SA, 80 mmol), 6.4 mL of pyridine (80 mmol), and 100 mL of chloroform were added to a round bottom flask (250 mL). The solution was refluxed in a 75-85° C. oil bath with Ar purging for overnight. The reaction mixture was allowed to cool to room temperature and 100 mL of chloroform was added. The mixture was washed successively with 100 mL each of 12.1 mM HCl, saturated NaCl, and deionized water. The organic layer was dried over magnesium sulfate and then the volume of the mixture was reduced by half by rotary evaporator. After pouring the mixture into 800 mL of a 1:1 hexane and diethyl ether, the polymer was allowed to precipitate at 4° C. for overnight. The polymer was collected and dried under vacuum to yield 8.1 g of PCL1.25 k-diSA. ¹H NMR (400 MHz, DMSO/TMS): δ 12.2 (s, 1H, COOH—), 4.1 (s, 2H, PCL-CO—CH₂—CH₂—COOH—) 4.0 (s, 12H, O—(CO—CH₂—(CH₂)₄—O)₆CO—CH₂—CH₂—COOH), 3.6 (s, 2H, PCL-CO—CH₂—CH₂—COOH—) 3.3 (s, 2H, —CH₂-PCL₆-SA), 2.3 (t, 12H, O—(CO—CH₂—(CH₂)₃—CH₂—O)₆CO—CH₂—CH₂—COOH), 1.5 (m, 24H, O—(CO—CH₂—CH₂—CH₂—CH₂—CH₂—O)₆CO—CH₂—CH₂—COOH), 1.3 (m, 12H, O—(CO—CH₂—CH₂—CH₂—CH₂—CH₂—O)₆CO—CH₂—CH₂—COOH). Similarly, PCL2k-diSA was synthesized using the procedure with 2,000 MW PCL-diol.

### Example 3

### Synthesis of PCL2k-diGly

10 g of polycaprolactone-diol (5 mmole, MW 2000) with 2.63 g of Boc-Gly-OH (15 mmole) was dissolved in 60 mL chloroform and purged with argon for 30 minutes. 3.10 g of DCC (15 mmole) and 61.1 mg of DMAP (0.5 mmole) were added to the reaction mixture and the reaction was allowed to proceed overnight with argon purging. The solution was filtered into 400 mL of diethyl ether along with 40 mL of chloroform. The precipitate was collected through filtration and dried under vacuum to yield 4.30 g of PCL2k-di-BocGly. ¹H NMR (400 MHz, CDCl₃/TMS): δ 5.1 (s, 1H, CH₂NHCOOC(CH₃)₃—), 4.2 (t, 2H, CH₂NHCOOC(CH₃)₃—) 4.0 (t, 16H, O—(CO—CH₂—(CH₂)₃CH₂—O)₈CO—CH₂—CH₂—COOH), 3.8 (t, 2H, O—CH₂CH₂—O—CO-PCL), 3.6 (t, 2H, O—CH₂CH₂—O—CO-PCL), 2.3 (t, 16H, O—CH₂CH₂—O—CO—CH₂(CH₂)₄—OCO), 1.7 (m, 32H, O—CH₂CH₂—O—CO—CH₂CH₂CH₂CH₂CH₂—OCO), 1.5 (s, 9H, CH₂NHCOOC(CH₃)₃), 1.3 (m, 16H, O—CH₂CH₂—O—CO—CH₂CH₂CH₂CH₂CH₂—OCO).

A Boc protecting group on PCL2k-di-BocGly was removed by reacting the polymer in 14.3 mL of chloroform and 14.3 mL of trifluoroacetic acid for 30 minutes. After precipitating twice in ethyl ether, the polymer was dried under vacuum to yield 3.13 g of PCL2k-diGly. ¹H NMR (400 MHz, CDCl₃/TMS): δ 4.2 (m, 4H, CH₂NH₂—) 4.0 (t, 16H, O—(CO—CH₂—(CH₂)₃CH₂—O)₈CO—CH₂—CH₂—COOH), 3.8 (t, 2H, O—CH₂CH₂—O—CO-PCL), 3.6 (t, 2H, O—CH₂CH₂—O—CO-PCL), 2.3 (t, 16H, O—CH₂CH₂—O—CO—CH₂(CH₂)₄—OCO), 1.7 (m, 32H, O—CH₂CH₂—O—CO—CH₂CH₂CH₂CH₂CH₂—OCO), 1.3 (m, 16H, O—CH₂CH₂—O—CO—CH₂CH₂CH₂CH₂CH₂—OCO). PCL1.25 k-diGly was synthesized using a similar procedure while using 1,250 MW PCL-diol.

### Example 4

### Synthesis of Medhesive-054

5 grams of 4-arm PEG-Amine-10k (0.5 mmole) was dissolved in 20 mL of DMF with 0.625 grams of PCL 1250-diSA (0.5 mmole), and 0.228 g of DOHA (1.25 mmole) in a round bottom flask. To this mixture, HOBt (0.338 grams; 2.5 mmole), HBTU (0.95 grams; 2.5 mmole), and Triethylamine (280 uL; 2.0 mmole) in 20 mL of chloroform and 30 mL of DMF was added dropwise over 60 minutes. After the reaction mixture was stirred for 2 hours, 0.0455 g of DOHA (0.25 mmole) was added and the mixture was further stirred at room temperature for 1 hour. This solution was filtered into diethyl ether and allowed to precipitate at 4° C. for overnight. The precipitate was collected by vacuum filtration and dried under vacuum for 24 hours. The polymer was dissolved in 75 mL of 50 mM HCl and 75 mL of methanol and dialyzed in 4 L of water (acidified to pH 3.5) for 2 using a 15,000 MWCO tube. 3.8 g of Medhesive-054 was obtained after lyophilization. ¹H NMR (400 MHz, DMSO/TMS): δ 8.7-8.5 (s, 1H, C₆H₃(OH)₂—), 7.9 (d, 2H, C₆H₃(OH)₂—), 6.5 (dd, 1H, C₆H₃(OH)₂—), (dd, 1H, C₆H₃(OH)₂—CH₂—CH₂—CONH—CH₂—CH₂—O—), 4.1 (s, 2H, PCL-CO—CH₂—CH₂—COOH—) 4.0 (s, 12H, O—(CO—CH₂—(CH₂)₄—O)₆CO—CH₂—CH₂—COOH), 3.6 (s, 2H, PCL-CO—CH₂—CH₂—COOH—) 3.3 (s, 2H, —CH₂-PCL₆-SA), 2.3 (t, 12H, O—(CO—CH₂—(CH₂)₃—CH₂—O)₆CO—CH₂—CH₂—COOH), 1.5 (m, 24H, O—(CO—CH₂—CH₂—CH₂—CH₂—CH₂—O)₆CO—CH₂—CH₂—COOH), 1.3 (m, 12H, O—(CO—CH₂—CH₂—CH₂—CH₂—CH₂—O)₆CO—CH₂—CH₂—COOH). UV-vis spectroscopy: 0.22±0.020 mole DH/mg polymer (3.6±0.33 wt % DH). GPC: Mw=98,000; Mn=35,000; PD=2.8. (DH=DOHA)

### Example 5

### Synthesis of Medhesive-061 (PEG20k-(DMu)₈)

50 g of 8-armed PEG-OH (20,000 MW; 20 mmol —OH) was dried via azeotropic evaporation of toluene, followed by drying in a vacuum dessicator. PEG was redissolved in 400 mL toluene, then a53 mL of phosgene solution (20% phosgene in toluene; 100 mmol phosgene) was added. The mixture was stirred at 55° C. for 4 hours with a NaOH solution trap to trap escaped phosgene. Toluene was evaporated and dried with vacuum overnight. 350 mL of chloroform and 3.46 g of N-hydroxysuccinimide (30 mmol) was added to the phosgene-activated PEG, followed by the addition of 4.18 mL (30 mmol) of triethylamine in 30 mL chloroform dropwise. The mixture was stirred under Argon for 4 hours. To the reaction mixture, a7.58 g dopamine-HCl (40 mmol), 11.16 mL triethylamine (80 mmol) and 120 mL DMF were added, then reaction was stirred at room temperature for overnight. The reaction mixture was added to diethyl ether, then the precipitate was collected via filtration and dried. The crude product is then purified further using dialysis (3500 MWCO) in deionized water (acidified to pH 3.5) for 24 hours. PEG20k-(DMu)₈[Medhesive-061] ¹H NMR (400 MHz, DMSO/TMS): δ 8.73-8.63 (d, 2H, C₆H₃(OH)₂—), 7.2 (m, 1H, PEG-C(O)—NH—), 6.62-6.42 (m, 3H, C₆H₃(OH)₂—), 4.04-4.02 (s, 2H, PEG-CH₂—O—C(O)—NH—), 3.68 (m, 2H, C₆H₃(OH)₂—CH₂—CH₂—NH—C(O)—O—), 3.62-3.41 (m, PEG), 3.07 (m, 2H, C₆H₃(OH)₂—CH₂—CH₂—NH—C(O)—O—). UV-vis spectroscopy: 0.375±0.01 μmole DM/mg polymer (6.84±0.18 wt % DM).

### Example 6

### Synthesis of Medhesive-096

C10 g of 10K, 4-arm PEG-OH (1 mmole) was combined with toluene (180 mL) in a 500 mL round bottom flask equipped with a condenser, Dean-Stark Apparatus and Argon inlet. While purging with argon, the mixture was stirred in a 140-150° C. oil bath until 90 mL of toluene was removed. The reaction was cooled to room temperature and 10.6 mL (20 mmole) of the 20% phosgene solution in toluene was added. The mixture was further stirred at 50-60° C. for 4 hours while purged with argon while using a 20 Wt % NaOH in a 50/50 water/methanol trap. Toluene was removed via rotary evaporation with a 20 Wt % NaOH solution in 50/50 water/methanol in the collection trap. The polymer was dried under vacuum for overnight. 691 mg (6 mmole) of NHS and 65 mL of chloroform was added to PEG and the mixture was purge with argon for 30 minutes. 840 μl (6 mmole) of triethylamine in 10 mL chloroform was added dropwise, and the reaction mixture was stirred with argon purging for 4 hours. After which, 427 mg (2.2 mmole) of dopamine hydrochloride in 25 mL of DMF and 307 μl (2.2 mmole) of triethylamine was added and the mixture was stirred for 4 hours. 2.4 g (1 mmole) of PCL-Gly along with 280 uL (2 mmole) of triethylamine was added and the mixture was further stirred for overnight. 133 mg (0.7 mmole) of dopamine hydrochloride was added to cap the reaction along with 98 μl (0.7 mmole) of triethylamine. The mixture was precipitated in ethyl ether and the collected precipitated was dried under vacuum. The crude polymer was dissolved in 150 mL of methanol and 100 mL 50 mM HCl and dialyzed (15000 MWCO dialysis tubing) in 4 L of water at pH 3.5 for 2 days with changing of the water at least 4 times a day. Lyophilization yielded the product. ¹H NMR (400 MHz, DMSO/TMS): δ 8.7-8.5 (s, 1H, C₆H₃(OH)₂—), 7.6 (t, 1H, -PCL-O—CH₂—CH₂—NHCOO—CH₂—CH₂—O—)), 7.2 (t, 1H, —O—CH₂—CH₂—NHCOO—CH₂—CH₂—C₆H₃(OH)₂), 6.7 (d, 1H, C₆H₃(OH)₂—), 6.5 (s, 1H, C₆H₃(OH)₂—), 6.4 (s, 1H, C₆H₃(OH)₂—), 4.0 (t, 16H, O—(CO—CH₂—(CH₂)₃CH₂—O)₈CO—CH₂—CH₂—COOH), 3.5 (m, PEG, —O—CH₂—CH₂—O—), 2.3 (t, 16H, —O—CH₂CH₂—O—CO—CH₂(CH₂)₄—OCO—), 1.7 (m, 32H, —O—CH₂CH₂—O—CO—CH₂CH₂CH₂CH₂CH₂—OCO—), 1.3 (m, 16H, —O—CH₂CH₂—O—CO—CH₂CH₂CH₂CH₂CH₂—OCO—); DH Wt %=2.34%; PCL Wt %=20.7%. UV-vis spectroscopy: 0.211±0.069 mole DH/mg polymer (2.92±0.34 wt % DH). GPC: Mw=65,570; Mn=14,850; PD=4.4.

### Example 7

### Synthesis of Medhesive-104

1.02 g of PCL2k-diSA (0.46 mmole) was dissolved with 5 g of, 10k, 4-arm-PEG-NH₂ (0.5 mmol) and 0.228 g of DOHA (1.25 mmol) in a 250 mL round bottom flask containing 20 mL of DMF. 0.338 g (2.5) of HOBt, 0.95 g (2.5 mmol) HBTU, and 280 uL (2 mmole) of triethylamine was dissolved in 35 mL of DMF followed by the addition of 20 mL of chloroform. The HOBt/HBTU/TEA solution was added dropwise over a period of 40 minutes. This was then allowed to stir for an additional 2 hours. A second addition of 0.045 g (0.25 mmol) of DOHA was added to the solution and allowed to react for an addition 30 minutes. The solution was filtered into diethyl ether, placed at 4 C for 24 hours to filter the precipitate and dried in a dessicator for an additional 24 hours. The polymer was dissolved in 75 mL of 100 mM HCl and 100 mL of MeOH. The solution was filtered using coarse filter paper and dialyzed (15000 MWCO dialysis tubing) in 4 L of water at pH 3.5 for 2 days with changing of the water at least 4 times a day. Lyophilization yielded the product. ¹H NMR (400 MHz, DMSO/TMS): δ 8.7-8.5 (s, 1H, C₆H₃(OH)₂—), 7.9 (d, 2H, C₆H₃(OH)₂—), 6.5 (dd, 1H, C₆H₃(OH)₂—), (dd, 1H, C₆H₃(OH)₂—CH₂—CH₂—CONH—CH₂—CH₂—O—), 4.1 (s, 2H, PCL-CO—CH₂—CH₂—COOH—) 4.0 (s, 16H, O—(CO—CH₂—(CH₂)₄—O)₆CO—CH₂—CH₂—COOH), 3.6 (s, 2H, PCL-CO—CH₂—CH₂—COOH—) 3.3 (s, 2H, —CH₂-PCL₆-SA), 2.3 (t, 16H, O—(CO—CH₂—(CH₂)₃—CH₂—O)₆CO—CH₂—CH₂—COOH), 1.5 (m, 32H, O—(CO—CH₂—CH₂—CH₂—CH₂—CH₂—O)₆CO—CH₂—CH₂—COOH), 1.3 (m, 16H, O—(CO—CH₂—CH₂—CH₂—CH₂—CH₂—O)₆CO—CH₂—CH₂—COOH); DH Wt %=1.17%; PCL Wt %=27.5%. UV-vis spectroscopy: 0.091±0.009 mole DH/mg polymer (1.49±0.15 wt % DH).

### Example 8

### Synthesis of Medhesive-105

40 g of 10K, 4-arm PEG-OH (4 mmole) was combined with toluene (240 mL) in a 500 mL round bottom flask equipped with a condenser, Dean-Stark Apparatus and Argon inlet. While purging with argon, the reaction was heated to 140-150° C. and stirred until half the volume has been removed. The reaction was cooled to room temperature. 42.4 mL (80 mmole) of the phosgene solution was added using a syringe. The mixture was stirred at 50-60 C for 4 hours while purging with argon using a 20 Wt % NaOH in a 50/50 water/methanol trap. Toluene was removed via rotary evaporation with a 20 Wt % NaOH solution in 50/50 water/methanol in the collection trap and the mixture was dried under vacuum overnight.

2.77 g (24 mmole) of NHS was added to PEG followed by addition of 260 mL chloroform. The mixture was purged with argon for 30 minutes and 3.36 mL(24 mmole) of triethylamine in 40 mL chloroform added dropwise. The mixture was stirred with argon purging for 4 hours.

To PEG-NHS solution 1.71 g (8.8 mmole) of dopamine hydrochloride in 75 mL DMF along was added along with 1.23 mL (8.8 mmole) of triethylamine and allowed to react for 4 hours.

6.0 g (4.2 mmole) of PCL1250-(Gly)₂ was added along with 1.12 mL (8 mmole) of triethylamine and allowed to react for 16 hours. An additional 532 mg (2.8 mmole) was dissolved in 25 mL DMF along with 392 μL triethylamine and stirred for 3.5 hours. The reaction mixture was added to 1.6 L diethyl ether and place into 4° C. for overnight. The solution was suction filtered and dried under vacuum for several days. This was then dissolved in 600 mL of methanol and 400 mL 50 mM HCl. This was then filtered using coarse filter paper and dialyzed (15000 MWCo dialysis tubing) in 10.5 L of water at pH 3.5 for 2 days with changing of the water at least 4 times a day. The solution was then freeze dried and placed under a vacuum for 4-24 hours. After drying, ¹H NMR, GPC and UV-VIS were used to determine purity and coupling efficiency of the catechol. P(CL1.25EG10kb-g-DH2) [Medhesive-105] L/N 003281. NMR (400 MHz, DMSO/TMS): DH:PEG:PCL=2:1.23:1.09. UV-vis spectroscopy: 0.237±0.008 μmole DH/mg polymer (3.92±0.14 wt % DH). GPC: Mw=320,000 Da; PD=6.892

### Example 9

### Synthesis of HO-PCL-PEG(600)-PCL-OH

26.3 g of PEG-diol (43.8 mmol, MW 600) and 200 mL of toluene was added and the mixture and was heated in 155-165° C. oil bath with Argon purging until 50 mL of toluene was collected. 100 g of ε-caprolactone (876 mmol) was added and heated until 20 mL of toluene was evaporated. 1.135 μL (3.50 mmol) of tin(II) 2-ethylhexanoate was added. The mixture was stirred for another 20 hrs in a 155-165° C. oil bath with Argon purging. The clued polymer was purified by ether precipitation twice to yield 54.2 g of polymer. Based on ¹H NMR, each PCL block consists of 21.2 caprolactone units with the overall number average MW of the polymer calculated to be 5,400 Da.

### Example 10

### Synthesis of SA-PCL-PEG(600)-PCL-SA (Medhesive-112 Starting Material)

25 g of HO-PCL-PEG(600)-PCL-OH (MW ˜5400; 4.63 mmole) was added with 4.63 g of succinic anhydride (46.3 mmole) and 3.74 mL of pyridine (46.3 mmole) to chloroform (250 mL) in a round bottom flask (500 mL). The solution was refluxed at 75-85° C. in an oil bath with argon purging for 24 hours, allowed to cool to room temperature, and another 250 mL of chloroform added to the solution. The mixture was washed with 250 mL of 12.1 mM HCl, followed by 250 mL of saturated NaCl, followed by 250 mL of DI water. The solution was dried with magnesium sulfate for 24 hours. The magnesium sulfate was filtered with coarse filter paper and the volume of the filtrate reduced by half using the roto evaporator. The mixture was filtered into 4 L of a 1:1 mixture of hexane and diethyl ether and sat at 4° C. for 24 hours. The solution was suction filtered and allowed to dry under vacuum for 24 hours. The dried sample was weighed and dissolved in 250 mL of chloroform and precipitate into 2.4 L of a 1:1 mixture of hexane and diethyl ether and let sat at 4° C. for 24 hours. The solution was suction filtered, allowed to dry under vacuum for 24 hours, and weighed.

17.5 g of product from the previous reaction, along with 4.63 g of succinic anhydride (46.3 mmole) was dissolved in 500 mL of chloroform. 3.74 mL of pyridine (46.3 mmol) was added and the solution refluxed at 75-85° C. in an oil bath with argon purging for 18 hours. The reaction was allowed to cool to room temperature. The mixture was washed with 250 mL of 12.1 mM HCl, followed by 250 mL of saturated NaCl, followed by 250 mL of DI water. The solution was dried with magnesium sulfate for at least 24 hours. The magnesium sulfate was filtered with coarse filter paper and the volume of the filtrate reduced by half using the roto evaporator. The mixture was filtered into 3.6 L of a 1:1 mixture of hexane and diethyl ether and let sit at 4° C. for 24 hours. The solution was suction filtered, allowed to dry under vacuum for 24 hours and weighed. HOOC-PCL-PEG(600)-PCL-COOH L/N 004973. ¹H NMR (400 MHz, CDCl3): δ 4.1 (s, 2H, PCL-CO—CH₂—CH₂—COOH—) 4.0 (s, 42H, O—(CO—CH₂—(CH₂)₄—O)₂₁CO—CH₂—CH₂—COOH), 3.6 (s, 2H, PCL-CO—CH₂—CH₂—COOH—) 3.3 (s, 2H, —CH₂-PCL₂₁-SA), 2.3 (t, 42H, O—(CO—CH₂—(CH₂)₃—CH₂—O)₂₁CO—CH₂—CH₂—COOH), 1.5 (m, 24H, O—(CO—CH₂—CH₂—CH₂—CH₂—CH₂—O)₂₁CO—CH₂—CH₂—COOH), 1.3 (m, 12H, O—(CO—CH₂—CH₂—CH₂—CH₂—CH₂—O)₂₁ CO—CH₂—CH₂—COOH).

### Example 11

### Synthesis of Medhesive-112

21.43 grams of 4-arm PEG-Amine-10k (2.14 mmole) was dissolved in 100 mL of DMF and 45 mL of chloroform with 12 grams of HOOC-PCL-PEG(600)-PCL-COOH (2.14 mmole), and 0.977 g of DOHA (5.36 mmole) in a round bottom flask. HOBt (1.45 grams; 10.7 mmole), HBTU (4.06 grams; 10.7 mmole), and triethylamine (2.075 mL; 14.97 mmole) was dissolved in 85 mL of chloroform and 130 mL of DMF. The HOBt/HBTU/Triethylamine solution was added dropwise to the PEG/PCL/DOHA reaction over a period of 30-60 minutes. The reaction was stirred for 24 hours. 0.594 grams of DOHA (3.26 mmole) was added to the reaction and let it stir for 4 hour. This solution was filtered into 3.6 L of diethyl ether and placed at 4° C. for 16-24 hours. The precipitate was suction filtered and dried under vacuum for 16-24 hours. The polymer was dissolved in 400 mL of methanol and 120 mL of DMF, and dialyzed using 15000 MWCO dialysis tubing against 10 L of water acidified to pH 3.5 for 3 days. The acidified water was changed at least 4 times daily. The solution was then freeze dried and placed under a vacuum for 4-24 hours. After drying, ¹H NMR and UV-VIS were used to determine purity and coupling efficiency of the catechol. P(CL5.4(EG600)EG10kb-g-DH2) [Medhesive-112] L/N's 005504. ¹H NMR (400 MHz, DMSO/TMS): δ 8.7-8.5 (s, 1H, C₆H₃(OH)₂—), 7.9 (s, 1H, C₆H₃(OH)₂—CH₂—CH₂—CONH—CH₂—CH₂—O—), 7.8 (s, 1H, -PCL-COO—CH₂—CH₂—CONH—CH₂—CH₂—O—), 6.6 (d, 1H, C₆H₃(OH)₂—), 6.5 (s, 1H, C₆H₃(OH)₂—), 6.4 (d, 1H, C₆H₃(OH)₂—CH₂—CH₂—CONH—CH₂—CH₂—O—), 4.1 (s, 2H, PCL-CO—CH₂—CH₂—CONH—CH₂—CH₂—O-PEG) 4.0 (s, 84H, O—(CO—CH₂—(CH₂)₄—O)₂₁CO—CH₂—CH₂—CONH), 3.6 (m, 278H, PEG), 1.5 (m, 168H, O—(CO—CH₂—CH₂—CH₂—CH₂—CH₂—O)₂₁CO—CH₂—CH₂—CONH), 1.3 (m, 84H, O—(CO—CH₂—CH₂—CH₂—CH₂—CH₂—O)₂₁CO—CH₂—CH₂—CONH). NMR: Wt % DOHA=1.81%; Wt % PCL=24.7%. UV-vis spectroscopy: 0.124±0.002 μmole DH/mg polymer (2.05±0.03 wt % DH).

### Example 12

### Synthesis of HO-PLA-PEG(600)-PLA-OH

14.9 g of PEG-diol (24.8 mmol, MW 600) was azeotropically dried with rotary evaporation using 50 mL of toluene twice and dried with vacuum pump for overnight. 50 g of L-lactide (347 mmol) and 100 mL of toluene was added and the mixture was heated in 155-165° C. oil bath with Argon purging until 50 mL of toluene was collected. The mixture was allowed to cool for 10 min and then 643 μL (1.98 mmol) of tin(II) 2-ethylhexanoate was added. The mixture was stirred for another 24 hrs in a 155-165° C. oil bath with Argon purging. The clued polymer was purified by ether precipitation twice to yield 35.7 g of polymer. Based on ¹H NMR, each PLA block consists of 25.0 lactide unit with the overall number average MW of the polymer calculated to be 4,200 Da.

### Example 13

### Synthesis of SA-PLA-PEG(600)-PLA-SA

25 g of HO-PLA-PEG(600)-PLA-OH (MW 4,200; 6 mmole) with 11.91 g of succinic anhydride (119 mmole) and 9.63 mL of pyridine (119 mmole) was added to chloroform (250 mL) in a round bottom flask (500 mL). The solution was refluxed at 75-85° C. in an oil bath with Argon purging for 24 hours. The reaction was allowed to cool to room temperature and another 250 mL of chloroform was added to the solution. The mixture was washed with 250 mL of 12.1 mM HCl, followed by 250 mL of saturated NaCl, followed by 250 mL of DI water. The solution was dried with magnesium sulfate for 24 hours. The magnesium sulfate was filtered with coarse filter paper and the volume of the filtrate reduced by half using the roto evaporator. The mixture was filtered into 2.4 L of a 1:1 mixture of hexane and diethyl ether and let sit at 4° C. for 24 hours. The solution was suction filtered, allowed to dry under vacuum for 24 hours and weighed. SA-PLA-PEG(600)-PLA-SA L/N 005525. ¹H NMR (400 MHz, CDCl₃): δ 5.2 (q, 25H, (OCHCH₃CO)₂₅—), 4.3 (m, 2H, PLA-COO—CH₂—CH₂—O-PEG-) 3.7-3.6 (m, 56H, PLA-(O—CH₂—CH₂)₁₄-PLA), 2.7-2.6 (m, 4H, PLA-CO—CH₂—CH₂—COOH—) 1.6-1.5 (d, 75H, (OCHCH₃CO)₂₅—).

### Example 14

### Synthesis of Medhesive-116

45 grams of 4-arm PEG-Amine-10k (4.5 mmole) was dissolved in 180 mL of DMF with 19.8 grams of SA-PLA-PEG(600)-PLA-SA (4.5 mmole), and 2.05 g of DOHA (11.3 mmole) in a round bottom flask. HOBt (3.04 grams; 22.5 mmole), HBTU (8.53 grams; 22.5 mmole), and Triethylamine (4.356 mL; 31.4 mmole) were dissolved in 180 mL of chloroform and 270 mL of DMF. HOBt/HBTU/Triethylamine solution was added dropwise to the PEG/PCL/DOHA reaction over a period of 30-60 minutes. The reaction was stirred for 24 hours. 1.25 g of DOHA (6.8 mmole) was added to the reaction and stirred for 4 hour. This solution was filtered into 3.2 L of diethyl ether and place at 4° C. for 24 hours. The precipitate was suction filtered and dried under vacuum for 16-24 hours. The polymer was dissolved in 350 mL of DMF. Once completely dissolved, mL of methanol was slowly added. This was then placed in 15000 MWCO dialysis tubing and dialyzed in 20 L of water at pH 3.5 for 3 days with changing of the water at least 4 times a day. The solution was then freeze dried and placed under a vacuum for 4-24 hours. After drying, ¹H NMR and UV-VIS were used to determine purity and coupling efficiency of the catechol. P(LA4.2(EG600)EG10kb-g-DH2) [Medhesive-116] L/N's 003104. ¹H NMR (400 MHz, DMSO/TMS): δ 8.7-8.5 (s, 1H, C₆H₃(OH)₂—), 7.9 (s, 1H, C₆H₃(OH)₂—CH₂—CH₂—CONH—CH₂—CH₂—O—), 7.8 (s, 1H, -PLA-COO—CH₂—CH₂—CONH—CH₂—CH₂—O—), 6.6 (d, 1H, C₆H₃(OH)₂—), 6.5 (s, 1H, C₆H₃(OH)₂—), 6.4 (d, 1H, C₆H₃(OH)₂—CH₂—CH₂—CONH—CH₂—CH₂—O—), 5.2 (q, 50H, (PEG-(OCHCH₃CO)₂₅)₂—), 4.2 (s, 2H,NH—CH₂—CH₂—O-PEG-), 3.7-3.1 (m, 278H, PEG), 2.6-2.2 (m, 4H, -PLA-COO—CH₂—CH₂—CONH—CH₂—CH₂—O—), 2.6-2.2 (m, 4H, C₆H₃(OH)₂—CH₂—CH₂—CONH—CH₂—CH₂—O—), 1.6-1.5 (d, 150H, (PEG-(OCHCH₃CO)₂₅)₂—). ¹H NMR: 2.77 Wt % DOHA; 21.02 Wt % PLA. UV-vis spectroscopy: 0.147±0.004 μmole DH/mg polymer (2.43±0.07 wt % DH).

### Example 15

### Burst Strength and Lap Shear Testing

1.0 Molecular Weight Determination using Gel

Permeation Chromatography (GPC)

Molecular weight of polymers described herein were determined by gel permeation chromatography in concert with triple-angle laser light scattering on a Optilab® rEX (Wyatt Technology) refractive index detector and a miniDAWN™ TREOS (Wyatt Technology) triple-angle light scattering detector using Shodex-OH Pak columns (SB-804 HQ and SB-802.5 HQ) in a mobile phase of 50:50 mixture of methanol and phosphate buffered saline. For the molecular weight calculation, the experimentally determined reflective index (dn/dc) value of the polymer was used.

2.1. Materials Used

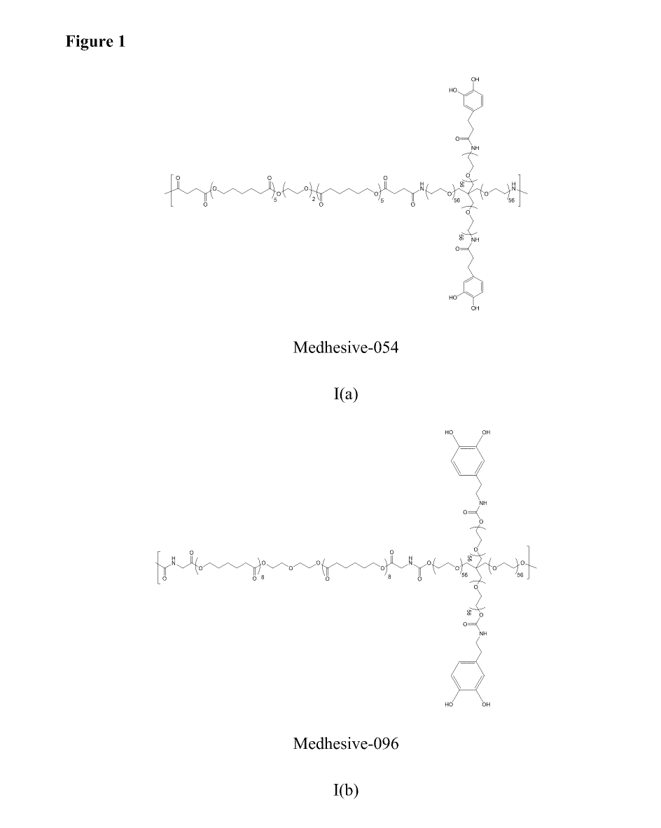

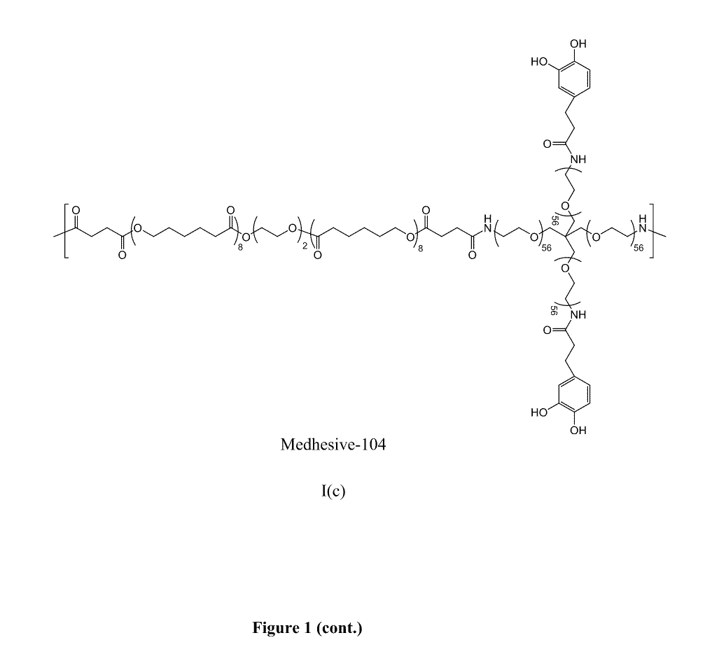

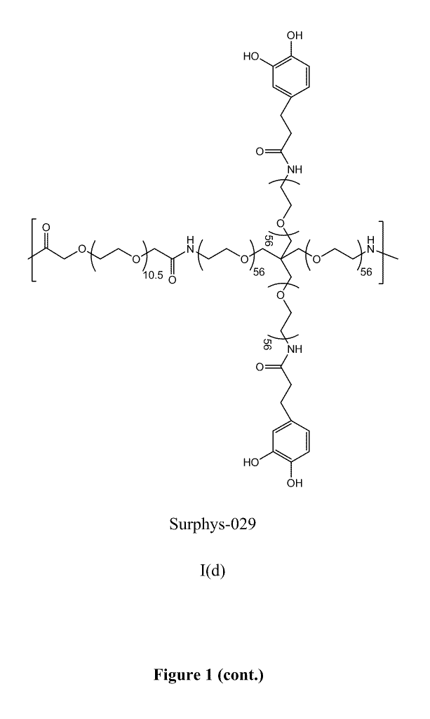

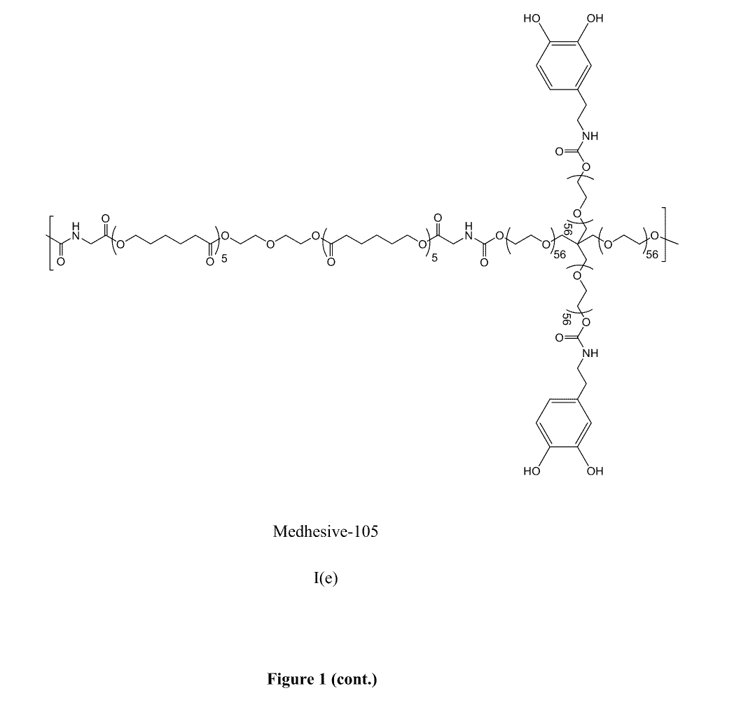

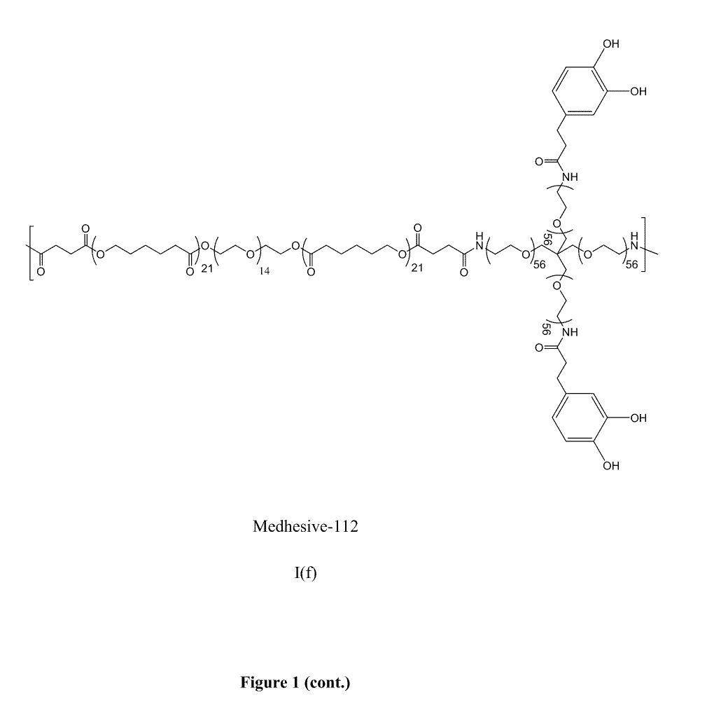

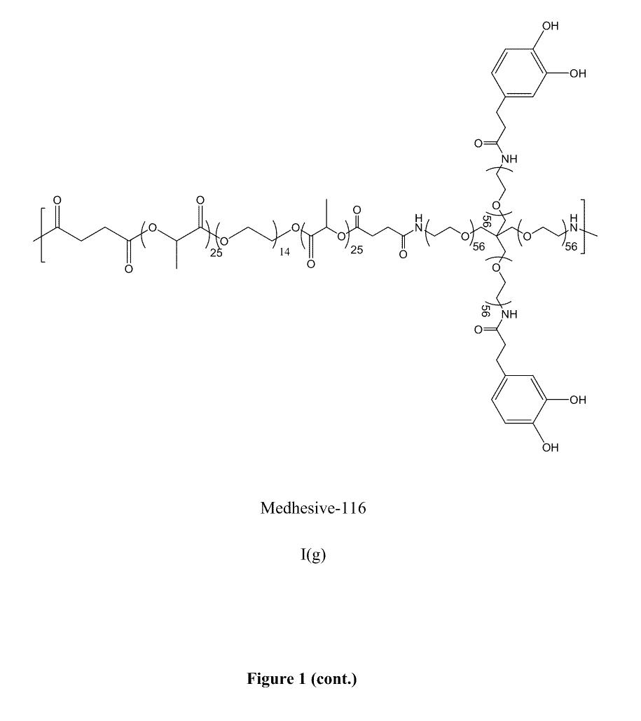

Medhesive-054 and Medhesive-096 were prepared as described above and their corresponding structure and composition can be seen in FIG. 1. ACS certified methanol and chloroform, along with 100×15 mm Fisherbrand petri dishes and concentrated phosphate buffered saline powder (diluted to 1× with 10 L of nanopure water) were obtained from Fisher Scientific. Bovine pericardium was obtained from Nirod Corporation, while the sodium periodate, 99.8+%, A.C.S. reagent was acquired from Sigma-Aldrich. A large number of 91×91 cm cover chip trays were purchased from Entegris, Inc. Poly(vinyl alcohol), 99+% hydrolyzed (89,000-98,000 MW) and poly(caprolactone)-diol (1250 MW) was purchased from Sigma-Aldrich, while poly(caprolactone)-triol (900 MW) was purchased from Acros.

2.2. Method for Coating Bioadhesive Polymer onto Bovine

Pericardium Backing for Burst Strength Testing

The bovine pericardium was cut so as to fit in an 88 mm diameter petri dish. Once placed inside the petri dish the bovine pericardium was flattened so that a smooth surface to coat was obtained and was placed in the fridge for 1 hour.

To the bovine pericardium was added ˜371 mg of Medhesive-054 or Medhesive-096, in 5 mL of methanol or 5 mL of chloroform, respectively, to obtain a coating thickness of ˜61 g/m². The variations in solvents were due to different solubility properties. Both bioadhesive polymers coated on bovine pericardium were then placed at 37° C. for 1 hour to remove most of the methanol or chloroform. This was then placed in the dessicator for at least 4 hours to ensure all methanol or chloroform was removed.

2.3. Method for Preparing Bovine Pericardium Defects for Burst Strength Testing

Bovine pericardium was cut into squares ˜40 cm in length and width and to these a 3 mm defect was punched in the center.

2.4. Preparation of Collagen Defects for Burst Strength Testing

A PTFE sheet was coated with a thin layer of petroleum jelly, to which, the bovine pericardium defect was placed on and smoothed out. Surgical gauze was then placed over the bovine pericardium defects so that the defects were allowed to stay hydrated but did not contain any excess moisture that could interfere with the adhesion of the bioadhesive-coated bovine pericardium backing.

2.5 Method of Preparing Bioadhesive-Coated Bovine Pericardium Sheets for Burst Strength Tests

Once the bioadhesive-coated bovine pericardium backing was dry it was cut into 10 mm circles. To the bovine pericardium defect was placed 31.7 uL of a 20 mg/mL solution of NaIO₄. The 10 mm circles of bioadhesive-coated bovine pericardium backing were then placed over the bovine pericardium defects. A glass plate was placed over the top of two of these substrates with the subsequent addition of a 100 gram weight to the top of the glass plate. After 2 hours the weight and glass plate are removed and the corresponding substrates were placed in PBS1× buffer for 1 hour at 37° C. Following this burst strength tests were performed with the results being reported in Section 3.1.

2.6. Method for Coating Bioadhesive Polymer onto Bovine Pericardium Backing for Lap Shear Testing.

The coating of the bovine pericardium backing with the bioadhesive polymer was performed in the following manner. The bovine pericardium was cut so as to fit in a 91×91 mm cover chip tray. Once placed inside the petri dish the bovine pericardium was flattened so that a smooth surface to coat was obtained.

To the bovine pericardium was added ˜505 mg of Medhesive-054 or Medhesive-096, in 5 mL of methanol or 5 mL of chloroform, respectively, to obtain a coating thickness of ˜61 g/m². The variations in solvents were due to different solubility properties. Both bioadhesive polymers coated on bovine pericardium were then placed at 37° C. for 1 hour to remove most of the methanol or chloroform. This was then placed in the dessicator for at least 4 hours to ensure all methanol or chloroform was removed.

The coating was cut in a 4×6 inch sheet of bovine pericardium and placed so that the middle portion was in a 1×4 inch groove. To this, 154 mg of the bioadhesive polymer in 2 mL of methanol and chloroform was poured on the surface and evaporated as described earlier, or they were coated as in Section 2.2. In addition a film applicator may be used to coat the backings.

2.7. Method for Preparing Bovine Pericardium Substrates for Lap Shear Testing

Bovine pericardium was cut into 1″×3″ rectangles.

2.8. Method of Preparing Bioadhesive-Coated Bovine

Pericardium Sheets for Lap Shear Tests

Once the bioadhesive-coated bovine pericardium backing was dry it was cut into 1×3 inch circles. To the bovine pericardium substrate was placed 40 uL of a 20 mg/mL solution of NaIO₄. The 1×3 inch bioadhesive-coated bovine pericardium backing was then placed over the 1×3 inch bovine pericardium substrates such that there was a 1 cm by 1 inch overlap for a total overlapping area of 0.000254 m². A glass plate was placed over the top of these substrates with the subsequent addition of a 100 gram weight to the top of the glass plate. After 2 hour the weight and glass plate were removed and the corresponding substrates were placed in PBS1× buffer for 1 hour at 37° C. Following this burst strength tests were performed with the results being reported in Section 3.2.

2.9. Method of Preparing Blended Bioadhesive/PCL-Coated Bovine Pericardium Sheets for Lap Shear Tests

The samples were prepared in the same fashion as described in Section 2.6 through 2.8. The major difference being that chloroform was used as a solvent and PCL-diol (MW=530) or PCL-triol (MW=900) was used along with Medhesive-054 at given weight percents. Medhesive-054 was placed at a coating weight of 61 g/m².

3.0. Method of Preparing Blended Bilayer Bioadhesive/PCL-Coated Bovine Pericardium Sheets for Lap Shear Tests

The samples were prepared as in Section 2.9, however, after the evaporation of chloroform a second addition of 50.5 mg of Medhesive-054 in 5 mL of water was added to the bovine pericardium. The water was evaporated off and the bilayer bioadhesive/PCL-coated bovine pericardium sheet was placed in the dessicator overnight.

3.1. Method of Preparing Blended Trilayer Bioadhesive/PCL-Coated Bovine Pericardium Sheets for Lap Shear Tests

To the bovine pericardium was added ˜50.5 mg of Medhesive-054 in 5 mL of water to obtain a coating thickness of ˜6.1 g/m². After this, the solvent was allowed to partially evaporate at 37° C., an addition of 505 mg and 252.5 mg of M-054 and PCL-triol, respectively, in chloroform was then added and the solvent was again allowed to evaporate off at 37° C. Following this, a third and final addition of 50.5 mg of Medhesive-054 in 5 mL of water was added and the solvent was once again allowed to evaporate off at 37° C. Once the solvent had been evaporated off the trilayer bioadhesive/PCL-coated bovine pericardium was placed in the dessicator overnight.

3.2. Method of Preparing Blended Bioadhesive/PVA-Coated Bovine Pericardium Sheets for Lap Shear Tests

PVA is insoluble in methanol and can only be dissolved through heating in water. Once dissolved in water it remains in solution at room temperature. In contrast, Medhesive-054 is relatively insoluble in water and soluble in methanol. If a solution of 2.5 mL of Medhesive-054 in methanol is placed in a solution of 2.5 mL of PVA in water the PVA precipitates out of solution. To combat this, PVA was dissolved in 1.25 mL of water through heating. After this, methanol was added in 0.25 mL increments with heating between each increment until the final volume was 2.5 mL. Medhesive-054 was subsequently dissolved in 1.25 mL of methanol. Once dissolved, water was added in 0.25 mL increments with sonication between each addition until the final concentration equaled 2.5 mL. If the two solutions are added together, PVA and Medhesive-054 begin to precipitate. To overcome this the PVA solution is added in 0.25 mL increments to the Medhesive-054 solution along with 0.25 mL of water with sonication after each addition. After these additions the final volume is 7.5 mL. This volume does not fully cover the surface area so water and methanol can be added in 0.25 mL increments to the solution such that the final volume is 10 mL with 6.25 mL being water and 3.75 mL being methanol. The solvent was then evaporated off at 37° C. and placed in the dessicator overnight.

3.3. Method of Preparing Blended Trilayer Bioadhesive/PVA-Coated Bovine Pericardium Sheets for Lap Shear Tests.

To the bovine pericardium was added ˜50.5 mg of Medhesive-054 in 5 mL of methanol to obtain a coating thickness of ˜6.1 g/m². This was then placed at 37° C. for 1 hour to remove most of the methanol. After this a second addition as described in section 3.2 was added. Once the solvent had been evaporated off a third and final addition of 50.5 mg of Medhesive-054 was added in 5 mL of water. The solvent was then evaporated off at 37° C. and placed in the dessicator overnight.

3.4. Method of Statistical Analysis

Statistical analysis was performed with SPSS using Oneway Anova by means of Post Hoc Testing using Tukey. All statistical analysis was performed at the 95% confidence interval with the positive control being Dermabond and the negative control being Tisseal in the case for lap shear testing. With burst strength testing the positive control was Dermabond and the negative control is Medhesive-061. For lap shear testing with blended and multi-layered formulations, Dermabond and the single-layered formulation of Medhesive-054 are used as the positive and negative control, respectively.

Results and Discussions

4.1. Burst Strength Testing

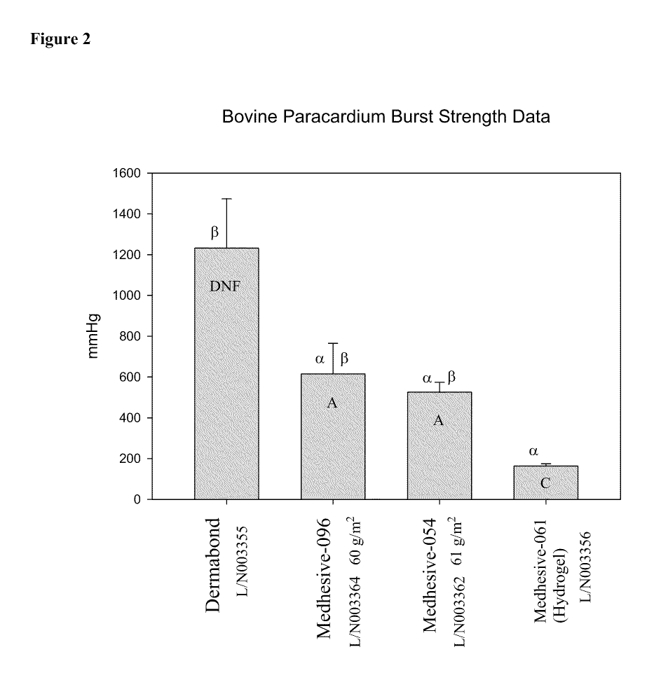

Results for burst strength testing of thin filmed bioadhesive-coated bovine pericardium backings showed performances 4 times better than normal catechol cross linked hydrogels (Medhesive-061) as shown in FIG. 2. However, when compared to Dermabond, there are significant differences in that Medhesive-054 and Medhesive-096 failed adhesively, while Dermabond did not break due to fear of breaking the burst strength tester, which was only accurate up to 800 mmHg.

4.2. Lap Shear Testing

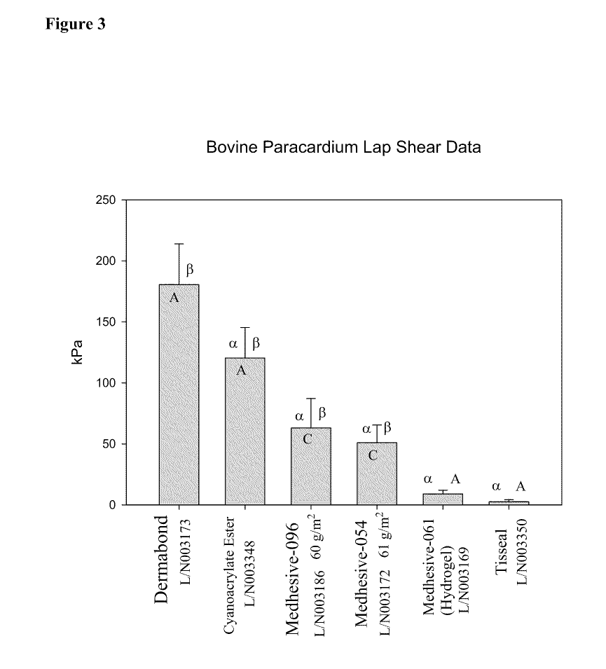

As shown in FIG. 3, lap shear adhesion strength of our thin-film bioadhesive performed 6-8 times better than adhesive hydrogels (Medhesive-061; failure at 8.9 kPa). Both Medhesive-054 and Medhesive-096 failed cohesively with the lap shear strength of 51 kPa and 63 kPa, respectively. Cyanoacrylate ester failed adhesively at 120 kPa while Dermabond performed the best, failing adhesively at 180 kPa. Tisseal performed the worst with a value of 2.6 kPa.

4.3. Lap Shear Testing on Blending Medhesive-054 with PCL

A blend of Medhesive-054 and either PCL-diol (MW=530) or PCL-triol (900 MW) were coated onto the pericardium and the maximum lap shear strength was determined. As shown in FIG. 4, PCL-diol did not increase the lap shear strength. However, lap shear strength increased with increasing PCL-triol content. At the highest concentration of PCL-triol tested (30 wt %), the formulation failed at the adhesive substrate interface as oppose to cohesive failure. The results here indicated that the cohesive properties of the adhesive film and the overall strength of the adhesive joint can be increased by incorporation of PCL-triol.

4.4. Lap Shear Testing Comparison of Blending Bilayer Formulations using PCL

Upon addition of the second coating a result was observed for the PCL blended formulations. FIG. 5 demonstrates that adding a second coating quadruples the peak stress value as compared to Medhesive-054 by itself. In addition, the primary mode of failure returned to a cohesive failure. Furthermore the statistical difference between the blended formulations from previous data were not statistically different than the control (Medhesive-054), however, the bilayer coating was statistically different.

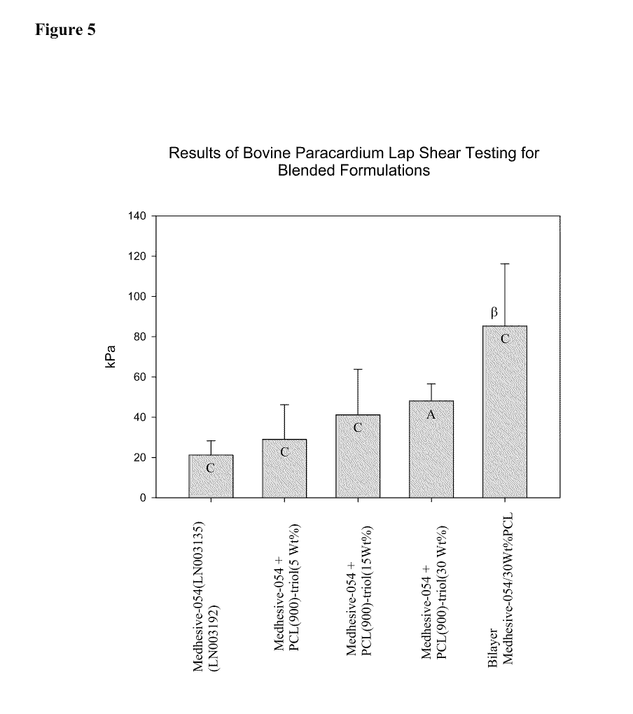

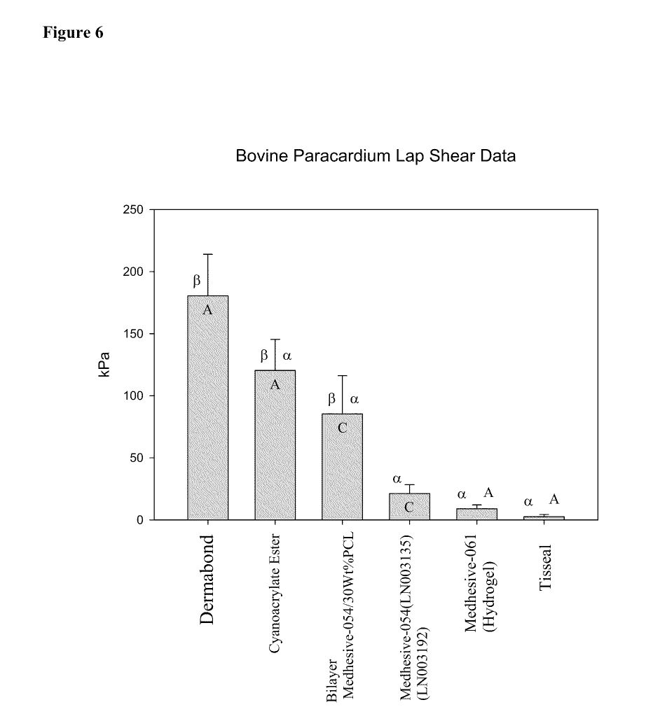

In FIG. 6, the data from FIG. 5 is compared to Medhesive-061 as the negative control and Dermabond as the positive control. These data points can be lumped into three distinct groups during statistical analysis which are as follows:

Group 1: Dermabond

Group 2: Cyanoacrylate Ester, Bilayer Medhesive-054/30 Wt % PCL

Group 3: Medhesive-054, Medhesive-061, and Tisseal

This data demonstrates that these blended bilayer formulations are statistically the same as cyanoacrylate ester, also known as crazy glue.

4.5. Lap Shear Testing Comparison of Blending Trilayer Formulations using PVA and PCL

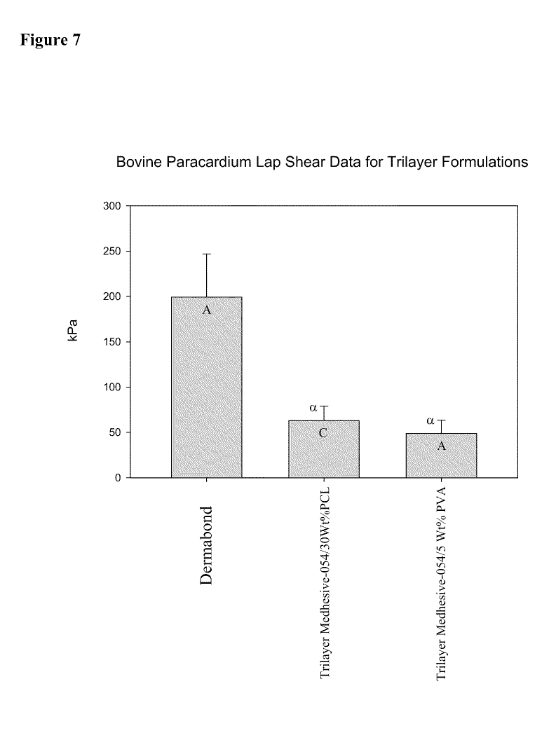

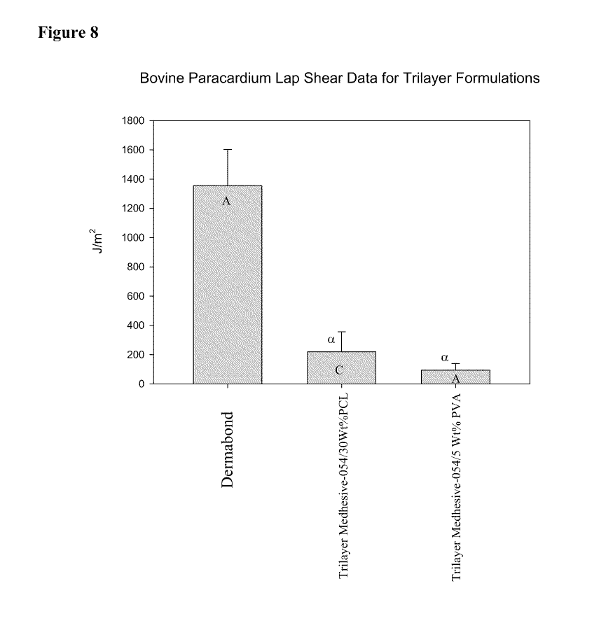

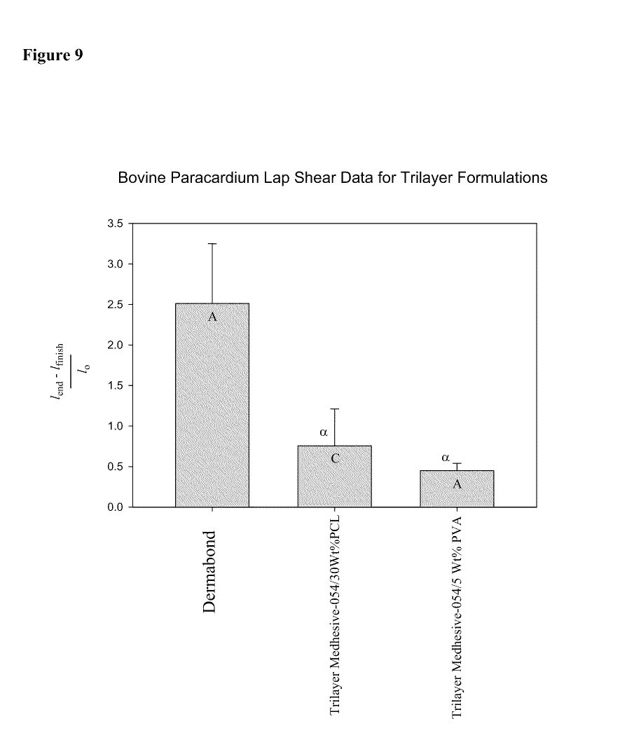

The data shown in FIGS. 7, 8 and 9 demonstrates the influence of using a trilayer formulation with PCL-triol or PVA. All data was stopped when the adhesive had lost 99% of its strength meaning it was possible to accurately calculate the energy needed to break the adhesive bond as well as the failure strain. The results show that blended thin-film adhesives are statistically different than Dermabond in all categories. Trilayer of Medhesive-054/5 Wt % PVA failed at the bovine pericardium backing-adhesive interface. It may be possible to add more Medhesive-054 in the first coating to create better adhesion. Overall, the relative amounts with each layer should be optimized to achieve maximum adhesive and cohesive properties.

4.6. Lap Shear Testing Comparison of Blending Trilayer Formulations using PVA

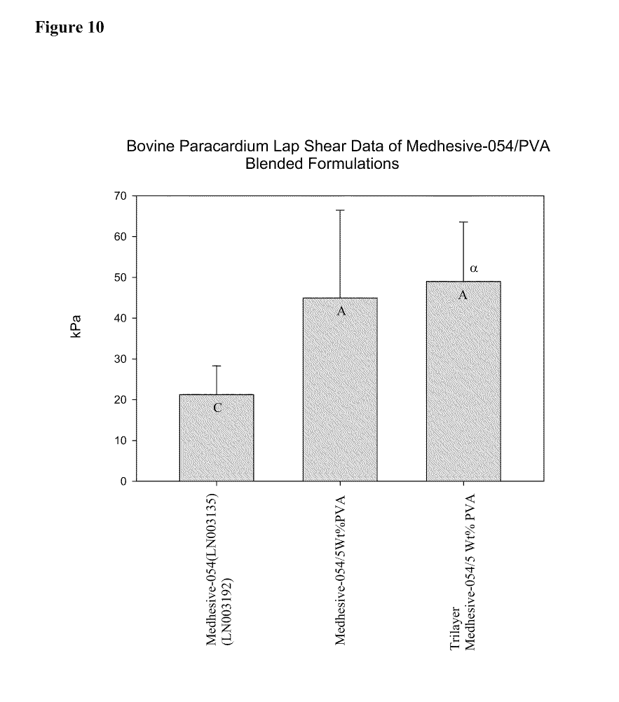

The results shown in FIG. 10 that the amount of stress that can be achieved is not statistically different for the trilayer blending versus the blending formulation. However, there is a statistical difference between the unblended Medhesive-054 and the trilayer coating. Improvements may be possible by increasing or decreasing the amount of Medhesive-054 or PVA in any of the three layers.

4.7 Burst Strength Tests performed using Strattice as Backing Material

72 mg of Medhesive-054 in 4 mL of methanol was coated onto Strattice, a dermal allograft from Lifecell corporation, and dried. Burst strength test was performed as specified above, using bovine pericardium as the test substrate. A burst pressure of 326+/−54 mmHg was recorded.

### Example 16

### Results for Surphys-029

Formation of Surphys-029 Hydrogel

Surphys-029 was dissolved in phosphate buffered saline (PBS, pH 7.4, at two times the normal concentration) at 300 mg/mL. The polymer solution was mixed with equal volume of NaIO₄ (12-48 mM) solution in a test tube lightly agitated. When the polymeric solution ceased to flow, the solution was considered fully cured. Table 1 shows that the minimum curing time occurs at around 28 seconds at a periodate:DOHA molar ratio of 0.33 to 0.5. This result demonstrated that Surphys-029 can cure rapidly and can potentially be used as an in situ curable tissue adhesive or sealant.

TABLE 1

| Periodate:DOHA | |
|---|---|
| Molar Ratio | Curing Time (s) |
| 1.00 | 150 |
| 0.75 | 50 |
| 0.66 | 37 |
| 0.50 | 28 |
| 0.33 | 28 |
| 0.25 | 38 |

5.3 Surphys-029 as an Anti-Fouling Coating

The substrate surfaces were cleaned by 10 minute sonication in 2-propanol. Test materials were coated with antifouling polymer by immersion in 1 mg/mL of Surphys-029 (0.3M K₂SO₄ 0.05M MOPS) for 24 hrs at 50° C. After coating, samples were rinsed twice with deionized water and dried in a stream of nitrogen gas.

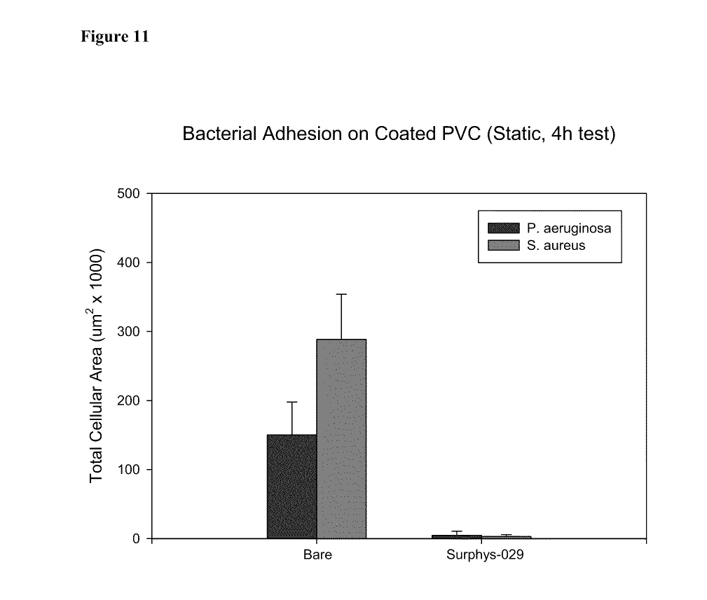

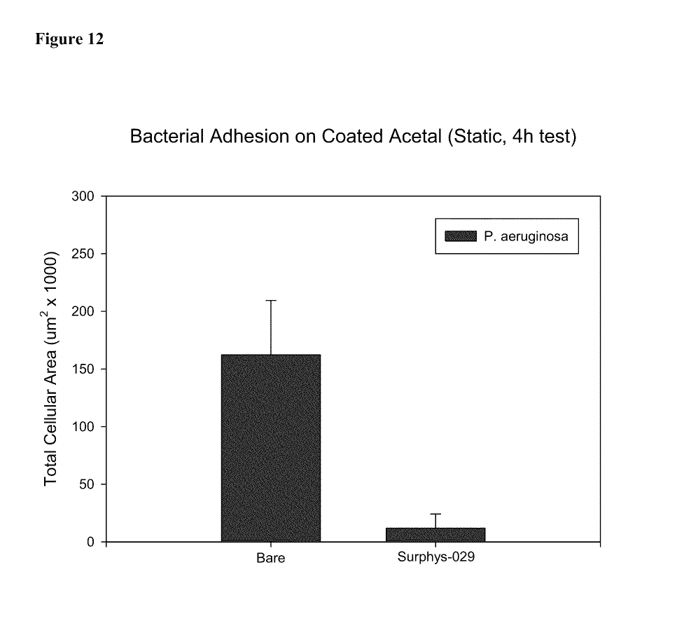

To determine the antifouling ability of these coatings, bacterial cell attachment and biofilm formation was assessed on both coated and uncoated samples. These surfaces were incubated with bacteria (1×10⁸ CFU/mL) in tryptic soy broth (TSB) in a 12-well cell culture plate for 4 hrs at 37° C. After which, each surface was rinsed three times with sterile PBS. The attached bacteria cells were stained with Syto 9 and 9 images per surfaces were capture using epifluorescence microscope. The total coverage of adhered bacteria cells on both PVC and acetal surfaces are shown in FIGS. 11 and 12. It was demonstrated that Surphys-029 coated surfaces demonstrated reduced the amount of bacteria attachment as compared to the uncoated surfaces.

### Example 17

### Coating of Adhesive Polymer onto Biologic Mesh

To coat the adhesive film onto bovine pericardium, a hydrated segment of biologic mesh was placed in a template of the same size (typically 91 mm×91 mm). A polymer solution (15-120 mg/mL) in methanol or chloroform was added and allowed to slowly evaporate in a 37° C. oven for at least one hour. The samples were further dried in a vacuum desiccator for at least 24 hours.

Burst Strength Adhesion Testing

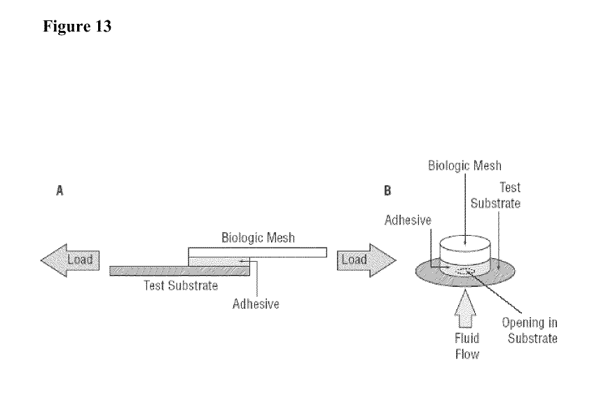

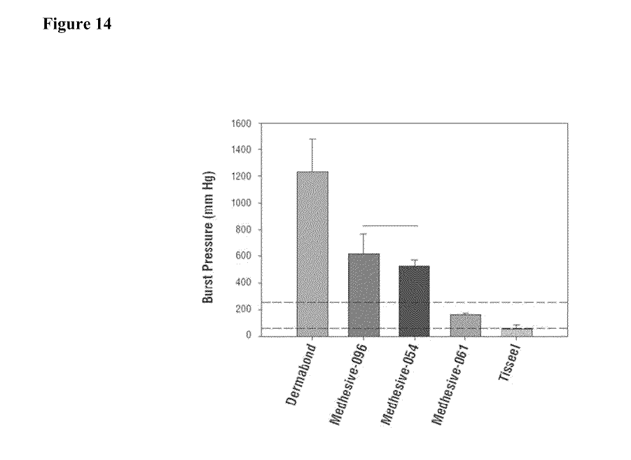

Procedures from American Society for Testing and Materials (ASTM) standards were used to perform burst strength (ASTM F2392) adhesion tests (FIG. 13). The adhesive coated-mesh was cut into 10-15 mm-diameter circular samples for burst strength tests. The test substrates (bovine pericardium) were shaped into 40 mm-diameter circles with a 3 mm-diameter defect at the center. A solution of NaIO₄ (40 μL) was added to the adhesive on the coated mesh prior to bringing the adhesive into contact with the test substrate. The adhesive joint was compressed with a 100 g weight for 10 min, and further conditioned in PBS (37° C.) for another hour prior to testing. A typical sample size was 6 in each test condition. Statistical assessment was performed using an analysis of variance (ANOVA), pair-wise comparisons with the Tukey test, and a significance level of 0.05. The adhesive properties of the bioadhesive constructs were determined and compared to controls: Dermabond®, Tisseel™, and Medhesive-061 (a Nerites liquid tissue adhesive). As seen in FIG. 14, Dermabond exhibited the highest adhesive strengths, and Medhesive-054 and Medhesive-096 significantly outperformed Medhesive-061 and Tisseel.

Lap Shear Adhesion Testing

Lap shear adhesion tests was performed using ASTM procedures (ASTM F2392) (FIG. 13). The adhesive coated-mesh was either cut into 2.5 cm×5 cm strips for lap shear tests. The test substrates (bovine pericardium) were shaped into 2.5 cm×5 cm strips. A solution of NaIO₄ (40 μL) was added to the adhesive on the coated mesh prior to bringing the adhesive into contact with the test substrate. The adhesive joint was compressed with a 100 g weight for 10 min, and further conditioned in PBS (37° C.) for another hour prior to testing. A typical sample size was 6 in each test condition. Statistical assessment was performed using an analysis of variance (ANOVA), pair-wise comparisons with the Tukey test, and a significance level of 0.05.

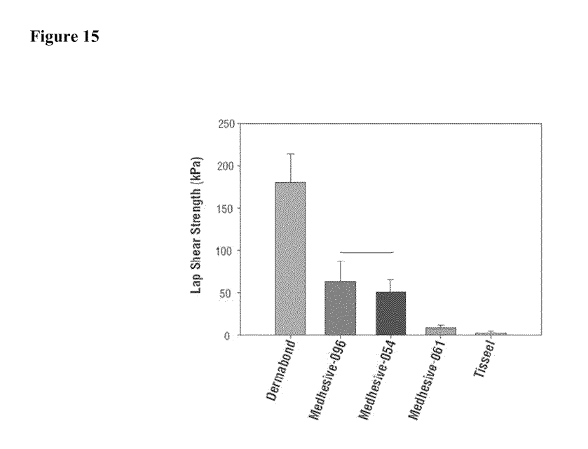

The adhesive properties of the bioadhesive constructs were determined and compared to controls: Dermabond®, Tisseel™, and Medhesive-061 (a Nerites liquid tissue adhesive). For both lap shear adhesion tests (FIG. 15), Dermabond exhibited the highest adhesive strengths, and Medhesive-054 and Medhesive-096 significantly outperformed Medhesive-061 and Tisseel.

### Example 18

### Effect of Periodate Concentration on Adhesive Properties

Using Medhesive-054 coated on bovine pericardium, NaIO₄ concentration was varied between 10-40 mg/mL. Lap shear adhesion test was performed as described above using bovine pericardium as the test substrate. As demonstrated in Table 2, both lap shear adhesion strength and work of adhesion, the total amount of energy required to separate the adhesive joint, increased with increasing NaIO₄ concentration, but exhibited no further increase when the concentration exceeded 20 mg/mL.

TABLE 2

| NaIO₄ | | Work of | |
|---|---|---|---|
| Concentration | Maximum | adhesion | |
| (mg/mL) | strength (kPa) | (J/m²)^{%} | Strain at Failure′ |
| 10 | 9.34 ± 2.89* | 22.2 ± 12.3^{$} | 0.489 ± 0.439 |
| 20 | 46.6 ± 19.3 | 77.0 ± 26.1^{$} | 0.366 ± 0.0698 |
| 30 | 42.3 ± 26.1 | 60.7 ± 34.5 | 0.315 ± 0.0627 |
| 40 | 45.0 ± 20.4 | 60.8 ± 14.6 | 0.168 ± 0.118 |

′Performed using Medhesive-054-coated bovine pericardium
^{%}Normalized by initial area of contact
*Significantly different from other test articles (p < 0.05)
^{$}Significantly different from each other (p < 0.05)

### Example 19

### Effect of Polymer Loading Density on Adhesive Properties

Using Medhesive-054 coated on bovine pericardium, the effect of polymer loading density (15-90 mg/mL) on the adhesive properties of the construct was determined. Lap shear adhesion test was performed as described above using bovine pericardium as the test substrate. As shown in Table 3, higher loading density yielded higher adhesive strengths for both lap shear and burst tests.

TABLE 3

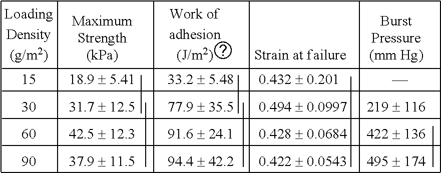

[symbol]

Performed using Medhesive-054-coated bovine pericardium [symbol]
Normalized by initial area of contact
Vertical lines = statistically equivalent; p > 0.05 [symbol]
indicates data missing or illegible when filed

### Example 20

### Effect of Contact Time on Adhesive Properties

Using Medhesive-054 coated on bovine pericardium, the effect of contact time on the adhesive properties of the construct was determined. Lap shear adhesion test was performed as described above using bovine pericardium as the test substrate. As demonstrated in Table 4, the adhesive joint had already reached maximum strength after merely 10 min of contact.

TABLE 4

| Contact | Percentage | Maximum Strength | |
|---|---|---|---|
| Time | Maximum Strength | Work of adhesion | Patterned Feature |
| (min) | (kPa) | (J/m²)^{%} | Strain at failure |
| 10 | 62.0 ± 23.2 | 89.4 ± 42.1 | 0.324 ± 0.137 |
| 70* | 60.6 ± 33.0 | 115 ± 43.6 | 0.479 ± 0.0892 |
| 120* | 55.7 ± 19.4 | 70.0 ± 21.5 | 0.332 ± 0.0361 |
| 180* | 58.2 ± 16.8 | 134 ± 79.9 | 0.518 ± 0.155^{$} |

[symbol]

Performed using Medhesive-054-coated bovine pericardium [symbol]
Normalized by initial area of contact
*Submerged in PBS at 37° C. for the final 60 min before testing
^{$}Statistically higher than 10-min contact time (p < 0.05) [symbol]
indicates data missing or illegible when filed

### Example 21

### Effect of Patterning on Adhesive Properties

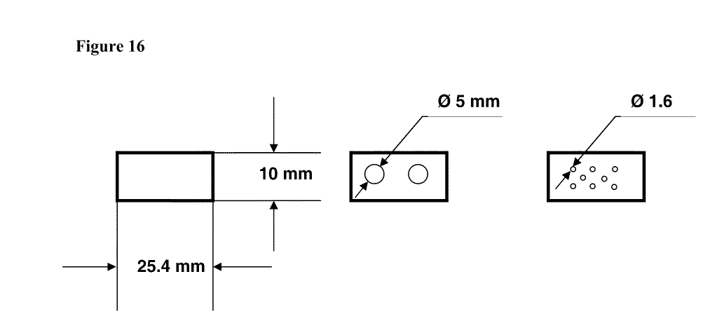

Medhesive-096 (60 g/m²) was coated on bovine pericardium with circular uncoated areas to determine the effect of patterning on the adhesive properties of the bioadhesive construct (FIG. 16). Lap shear adhesion test was performed as described above using bovine pericardium as the test substrate. As demonstrated in Table 5, the adhesive strength in general decreased with decreased areas of uncoated regions.

TABLE 5

| | | | |
|---|---|---|---|
| of Area | | | |
| Coated with | (kPa) | Diameter | Number of |

| Adhesive | Average | St Dev. | (mm) | Features |
|---|---|---|---|---|
| 100% | 107.5 | 24.7 | — | — |
| 95.5% | 86.6 | 13.3 | 1.6 | 8 |
| 84.5% | 70.0 | 8.10 | 5 | 2 |

### Example 22

### Effect of Oxidant Delivery Method on Adhesive Properties

The effect of different oxidant delivery methods was studied by testing lap shear adhesion strengths of Medhesive-054 (120 g/m²) coated on Permacol®. Lap shear adhesion test was performed as described above using bovine pericardium as the test substrate. For the brush method, a solution of 40 μL of 20 mg/mL of NaIO₄ was brushed onto the substrate prior to forming the adhesive joint. For the spray method, NaIO₄ solution (20 mg/mL) was sprayed on the construct prior to contact with the substrate. For the dipping method, the construct was dipped into a 20 mg/mL NaIO₄ solution prior to forming the adhesive joint. Results from the lap shear adhesion test can be seen in Table 6.

TABLE 6

| | Max Strength | | | | Work of | |
|---|---|---|---|---|---|---|
| Delivery | (kPa) | | Failure Strain | | Adhesion (J/m²) | |
| Method | Avg | St. Dev. | Avg | St. Dev. | Avg | St. Dev. |
| Brush | 71.0 | 12.2 | 0.406 | 0.114 | 128 | 71.7 |
| Spray | 94.2 | 4.19 | 0.352 | 0.0695 | 132 | 44.0 |
| Dip | 70.4 | 16.9 | 0.301 | 0.0692 | 89.2 | 44.8 |

### Example 23

### Adhesive Coated on Commercially Available Hernia Meshes

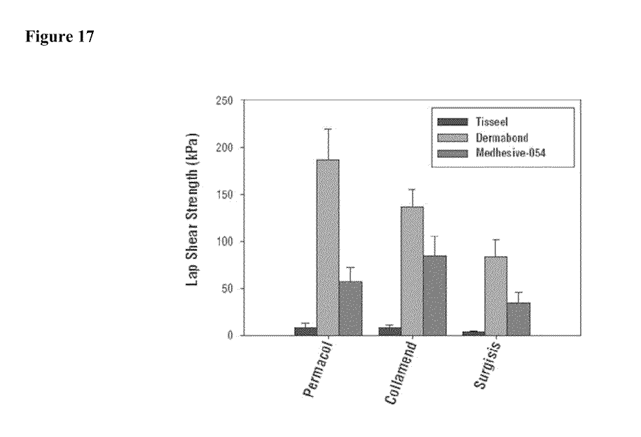

Three commercially available biologic meshes, two crosslinked porcine dermal tissues, Permacol™ and CollaMend™, and a multi-layered porcine small intestinal submucosal tissue, Surgisis™, were coated with Medhesive-054. Lap shear adhesion tests were performed using hydrated bovine pericardium as the test substrate. Although Dermabond exhibited the highest shear strength, meshes fixed with cyanoacrylate were reported to have reduced tissue integration combined with pronounced inflammatory response. Additionally, cyanoacrylate adhesive significantly reduced the elasticity of the mesh and abdominal wall, and impaired the biomechanical performance of the repair. Due to the release of toxic degradation products (formaldehyde), cyanoacrylates are not approved for general subcutaneous applications in the US. Medhesive-054 combined with all mesh types outperformed Tisseel by seven- to ten-fold (FIG. 17). Even with relatively weak adhesive strengths, fibrin-based sealants have demonstrated at least some level of success in mesh fixation in vivo, which suggests that bioadhesive constructs have sufficient adhesive properties for hernia repair. While the Medhesive-054 constructs only exhibited adhesive strengths that were 30-60% of those of Dermabond, it is possible to further optimize the coating technique or adhesive formulation for each mesh type.

### Example 24

### Effect of Sterilization on Adhesive Properties

Medhesive-054 (120 g/m²)-coated Permacol™ was sterilized with electron beam (E-beam) irradiation (15 kGy) and it adhesive properties was compared with a non-sterile construct. Lap shear adhesion test was performed as described above using bovine pericardium as the test substrate. As shown in Table 7, E-beam did not have any effect on the adhesive properties on the bioadhesive construct.

TABLE 7

| | Max Strength | | | | Work of | |
|---|---|---|---|---|---|---|
| Delivery | (kPa) | | Failure Strain | | Adhesion (J/m²) | |
| Method | Avg | St. Dev. | Avg | St. Dev. | Avg | St. Dev. |
| None | 71.0 | 12.2 | 0.406 | 0.114 | 128 | 71.7 |
| Sterile | | | | | | |
| E-beam | 86.3 | 35.3 | 0.361 | 0.0680 | 139 | 93.2 |
| Treated | | | | | | |

### Example 25

### Burst Strength of Adhesive Coated on Commercially Available Biologic Mesh

Burst strength adhesion test (ASTM F2392) was performed on Medhesive-054 (46 g/m²)-coated Strattice®, a porcine dermis mesh, using bovine pericardium as the test substrate. The average maximum pressure was found to be 326±54.4 mmHg.

### Example 26

### Adhesive Coated on Commercially Available Dural Meshes

Burst strength adhesion test (ASTM F2392) was performed on Medhesive-096 (90 g/m²)-coated SyntheCel®, a sheet formed from cellulose fibers, using bovine pericardium as the test substrate. The average maximum pressure was found to be 412.±78.9 mmHg.

### Example 27

### Sealing of Small Intestine

Bovine small intestines were rinsed and cut into 6″ segments. A small incision was made near the center with a #11 scalpel blade and sutured once with 5-0 nylon sutures. 37.1 uL of 20 mg/mL NaIO₄ solution was applied directly to the intestine around the defect and a 15 mm diameter bovine pericardium backing-coated with 60 g/m² of Medhesive-054 was applied over the defect. The adhesive joint was weighted down with a 100 g weight and allowed to cure for 10 min. The tissue was then hydrated for 1 hour in PBS at 37° C. and burst testing was performed by pumping air into the intestine at a rate of 20 mL/min until bubbles appeared from the defect. At which point the pressure was recorded. The average maximum pressure was found to be 49.4±19.2 mmHg.

### Example 28

### Adhesive Coated on a Synthetic Mesh

A polymer solution in methanol or chloroform (70-240 mg/mL) was added onto a fluorinated-release liner and dried in a vacuum desiccator. A synthetic mesh was placed over the dried film and two glass plates were used to sandwich the construct while being held in place with paper binders. The material was put into a desicator which was vacuumed and refilled with Ar gas. The dedicator was incubated at 55° C. for 1 hour and cooled to room temperature prior to use. Lap shear adhesion test (ASTM F2255) was performed using bovine pericardium as the test substrate. For Medhesive-096 (240 mg/mL) coated on a Dacron™ mesh, values for maximum lap shear strength, strain at failure, and work of adhesion were found to be 69.3±9.82 kPa, 0.516±0.0993, and 174±13.8 J/m², respectively, with n=5.

### Example 29

### Adhesive Coated on a Titanium Surface

Titanium (Ti)-coated silicon slides with a dimension of ½ in.×1½ in. were cleaned in four solvents 5% Contrad solution, H₂O, acetone, and isopropanol sequentially in a sonication bath and then treated with oxygen plasma for 5 minutes. 54.4 μl of Medhesive-096 (70 mg/mL) solution in chloroform was dropped onto the end of the Ti slide in a ½ ins×1 cm area, and the solvent were allowed to evaporate. 40 μl of 20 mg/ml of NaIO₄ was brushed onto one adhesive-coated slide and, which was brought into contact with another adhesive-coated slide to form an adhesive joint, which was weighted down by a 100 g weight for 2 hours. Lap shear adhesion test (ASTM D1002) was performed on the adhesive joint and values for maximum strength, strain at failure, and work of adhesion were found to be 307 kPa, 0.90, and 360 J/m², respectively.

### Example 30

### Effect of Blending on Adhesive Properties

To form an adhesive coating blend, Medhesive-054 with PCL-triol (MW=900, 0-30 wt %) was dissolved in methanol (60 g/m²) and coated onto bovine pericardium as previously described. Lap shear adhesion test was performed as described above using bovine pericardium as the test substrate. As shown in Table 8, both maximum lap shear strength and strain at failure did not change statistically. However, at elevated PCL-triol content (30 wt %), the work of adhesion was nearly doubled (p<0.05).

TABLE 8

| | Lap Shear | | | | Work of | |
|---|---|---|---|---|---|---|
| Wt % PCL- | Strength (kPa) | | Strain at Failure | | Adhesion (J/m²) | |
| triol | Avg. | St. Dev. | Avg. | St. Dev. | Avg. | St. Dev. |
| 0 | 70.0 | 9.50 | 0.293 | 0.0498 | 77.7 | 13.3 |
| 5 | 65.6 | 18.8 | 0.358 | 0.0519 | 99.4 | 15.6 |
| 15 | 88.4 | 20.1 | 0.469 | 0.191 | 117 | 15.8 |
| 25 | 61.3 | 20.3 | 0.410 | 0.100 | 95.9 | 35.3 |
| 30 | 74.6 | 29.3 | 0.481 | 0.160 | 131* | 51.2 |

### Example 31

### Effect of Blending on Adhesive Film Degradation

Adhesive films were incubated in PBS at 55° C. and their mass loss over time was recorded. Medhesive-054 films lost over 26.2±3.21 wt % of its original mass after 31 days of incubation. When blended with PCL-triol (30 wt %), mass loss was accelerated, demonstrating 34.5±3.73 wt % loss in only 24 days. However, blending with 5 wt % polyvinyl alcohol (PVA) did not result in changes in the rate of film degradation (22.5±1.11 wt % mass loss over 35 days).

### Example 32

### Adhesive Coated on a Synthetic Mesh

A polymer solution in methanol or chloroform (240 mg/mL) was added onto a fluorinated-release liner and dried in a vacuum dessicator. A synthetic mesh was placed over the dried film and two glass plates were used to sandwich the construct while being held in place with paper binders. The material was put into a dessicator which was vacuumed and refilled with Ar gas. The desicator was incubated at 55° C. for 1 hour and cooled to room temperature prior to use. Lap shear adhesion test (ASTM F2255) was performed using bovine pericardium as the test substrate and the lap shear strength and work of adhesion of construct coated on Dacron™ and polypropylene meshes are shown in Table 9.

TABLE 9

| Adhesive | | Maximum | Work of Adhesion |
|---|---|---|---|
| Polymer | Mesh Type | Strength (kPa) | (J/m²) |
| Medhesive-096 | Dacron | 69.3 ± 9.80 | 174 ± 13.8 |
| Medhesive-112 | Dacron | 44.2 ± 32.2 | 154 ± 128. |
| Medhesive-054 | Polypropylene | 46.0 ± 15.6 | 81.6 ± 47.8 |
| | PPKM404 | | |
| Medhesive-054 | Polypropylene | 45.6 ± 21.2 | 145 ± 33.6 |
| | PPKM602 | | |
| Medhesive-054 | Polypropylene | 26.1 ± 10.2 | 76.8 ± 35.6 |
| | PPKM802 | | |
| Medhesive-054 | Polypropylene | 30.3 ± 17.0 | 47.5 ± 32.3 |
| | PPKM802 | | |
| Medhesive-096 | Polypropylene | 33.9 ± 13.0 | 36.4 ± 19.1 |
| | PPKM802 | | |

### Example 33

### Patterned Adhesive Coating of Mesh for Accelerated Mesh-Tissue Integration

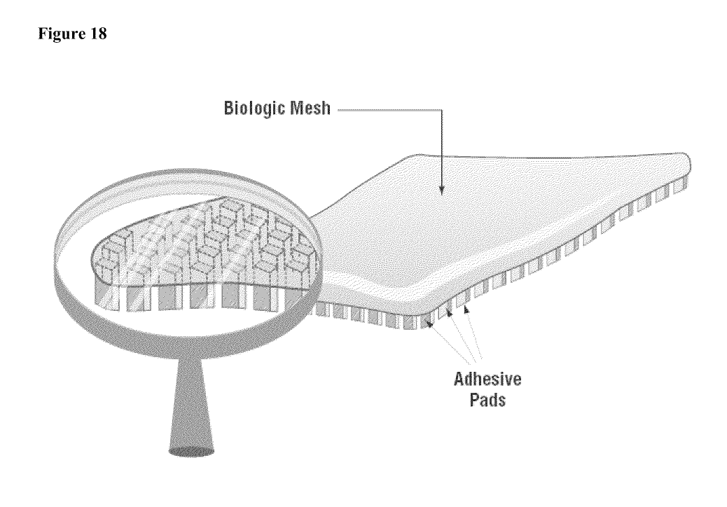

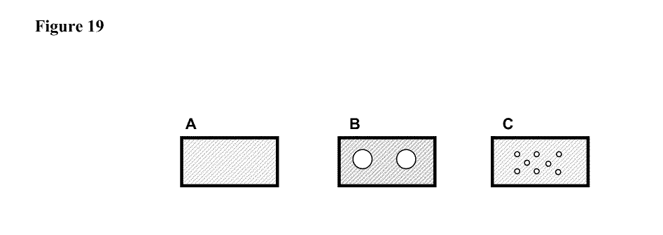

The adhesive polymer can be coated on the mesh in a pattern to promote faster integration of the host tissue and mesh. Unlike other fixation methods, adhesives may act as a barrier for tissue ingrowth into the mesh if their degradation rate is slower than the cell invasion rate and subsequent graft incorporation. Meshes secured with a slow degrading adhesive such as cyanoacrylate demonstrated impaired tissue integration. For meshes secured with conventional methods, the tensile strength of the mesh-tissue interface reached a maximum within four weeks after implantation, indicating that the meshes were fully integrated with the host tissue. This suggests that cellular infiltration occurs earlier. While the adhesive polymers of the invention exhibit a variety of degradation profiles, some formulations may take several months to be completely absorbed. To ensure rapid tissue integration into the mesh while maintaining strong adhesion at the time of implantation, adhesives can be coated onto a mesh in an array of adhesive pads, leaving other areas of the mesh uncoated as shown in FIG. 18. Other patterns with various geometric shapes (circular, rectangular, etc.) can also be created FIG. 19. The regions coated with adhesive will provide the initial bonding strength necessary to secure the mesh in place, while the uncoated regions will provide an unobstructed path for cellular invasion and tissue ingrowth to immediately occur.

To create a patterned adhesive polymer coating, a solvent casting method could be used, in which a metallic lattice will be placed over the mesh while the polymer solution is drying. The lattice will be used to displace the polymer solution so that an uncoated region is formed as the solution dries. By controlling the dimensions (5-10 mm) and the thickness (0.2-1.0 mm) of the lattice, it is possible to vary the ratio of the surface areas of the coated and uncoated regions. Bovine pericardium will be used both as the surrogate backing and test substrate. Lap shear adhesion testing will be performed to determine the effect of the patterned coating on the adhesive properties of the bioadhesive construct. For each coating pattern, a minimum of 10 repetitions will be tested, and statistical analysis will be performed using ANOVA, the Tukey post hoc analysis, and a significance level of p=0.05.

The adhesive strength of the patterned coating will likely be slightly lower compared to the non-patterned adhesive coating since the overall surface area of the adhesive is decreased. By varying the ratio of the surface areas between the coated and uncoated regions, the surface can be tailored adjust for the initial adhesive strength to the rate of tissue ingrowth. A pattern that results in greater than 80% of the adhesive strength of the non-patterned coating will be selected for subsequent animal studies. The rate of tissue ingrowth will be determined by implanting both patterned and non-patterned bioadhesive constructs into a rabbit model.

### Example 34

### Characterization of the Adhesive Polymer Films

Adhesive polymers were cast into films by the slow evaporation of methanol or chloroform in a mold (referred to as adhesive films in this proposal). Their percent swelling, tensile mechanical properties, and in vitro degradation profiles were then determined. For each test, the films were cured by the addition of a sodium periodate (NaIO₄) solution. Additionally, PCL-triol (30 wt %) was formulated into the adhesive film to determine the effect of added PCL content on the physical and mechanical properties of the adhesives. The equilibrium swelling of the adhesive films in phosphate buffered saline (PBS, pH 7.4, 37° C., 24 hours) was calculated by the equation, (Wₛ−Wᵢ)/Wᵢ where Wᵢ and Wₛ are the weights of the dry and swollen films measured before and after the swelling experiment, respectively. As shown in Table 10, the degree of swelling is affected by the composition of the adhesive formulation, as well as by the loading density (mass of polymer per unit area of the mold) of the films. For example, higher PCL content in Medhesive-096 (21 wt %) resulted in less swelling compared to Medhesive-054 (13 wt %). When PCL-triol was added to both polymers, these formulations exhibited significantly less swelling. The extent of water uptake is related to the hydrophobicity of the films. In addition to PCL content, the polymer loading density also affected the extent of swelling, with films formed with half the loading density absorbing 1.4 times more water. The loading density likely affected the crosslinking density of the film, which is inversely proportional to the degree of swelling.

TABLE 10

| | Loading | | Swollen film | Extent of |
|---|---|---|---|---|
| Adhesive | Density | Weight % | thickness | Swelling |
| Polymer | (g/m²) ^{#} | PCL | (□m) ^{$} | (Wₛ − Wᵢ/Wᵢ) * |
| Medhesive-054 | 23 | 0 | 263 ± 9.64 | 9.8 ± 0.90 |
| | 46 | 0 | 368 ± 4.58 | 7.2 ± 0.61 |
| | 46 | 30 | 260 ± 40.1 | 4.2 ± 0.50 |
| Medhesive-096 | 23 | 0 | 189 ± 4.51 | 7.0 ± 0.20 |
| | 46 | 0 | 261 ± 11.9 | 5.0 ± 0.20 |
| | 46 | 30 | 209 ± 6.66 | 4.2 ± 0.20 |

^{#} Amount of polymer used to form the dry film in mass per unit area of the mold;
^{$} Measured with micrometer;
* For each polymer type, the mean values for each test article are significantly different from each other (p < 0.05)

Determination of the tensile mechanical properties of the adhesives was based on American Society for Testing and Materials (ASTM) D638 protocols. Tensile tests on dog-bone shaped films (9.53 mm gauge length, 3.80 mm gauge width, and 12.7 mm fillet radius, swollen in PBS (pH 7.4) for 1 hr) were performed and the maximum tensile strength was measured. Both the Young's modulus and toughness were also determined, based on the initial slope and area under the stress-strain curve, respectively. As shown in Table 11, the mechanical properties of the film were affected by the PCL content. For example, Medhesive-096 demonstrated significantly higher tensile strength and toughness (251±21.2 kPa, and 266±29.1 kJ/m³, respectively), compared to Medhesive-054 (168±31.0 kPa and 167±38.6 kJ/m³). Strength and toughness values for Medhesive-096 formulated with the addition of 30 wt % of PCL-triol were even greater (357±37.5 kPa and 562±93.1 kJ/m³, respectively), suggesting that the mechanical properties of these adhesives can be modulated by blending them with compounds that impart the desired characteristics. The toughness more than doubled with the addition of PCL-triol to Medhesive-096. Elevated film toughness has been found to strongly correlate to high lap shear adhesion strength. Addition of PCL-triol probably increased the crosslinking density in the film, which resulted in the observed increase in mechanical properties. This increase in crosslinking density did not result in brittle films as shown in the elevated strain values.

TABLE 11

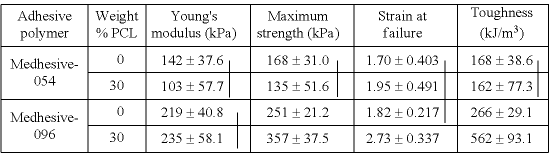

Vertical lines = statistically equivalent; p > 0.05

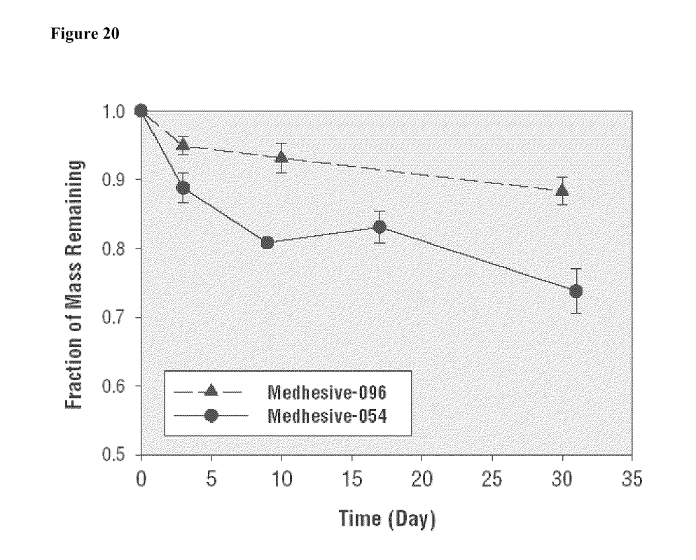

The in vitro degradation was determined by monitoring the mass loss of the adhesive films incubated in PBS (pH 7.4) over time at 55° C. to accelerate the degradation process (FIG. 20). Medhesive-054 lost over 26±3.2% of its original dry mass over one month, while the more hydrophobic Medhesive-096 demonstrated a slower rate of degradation (12±2.0% mass loss). Hydrolysis was also performed at 37° C. where these films lost over 13±2.9% (Medhesive-054) and 4.0±2.3% (Medhesive-096) after 18 and 20 days of incubation, respectively. Since adhesive films degrade mainly through hydrolysis, more water uptake by Medhesive-054 films (collaborated with elevated swelling) resulted in faster degradation.

These results demonstrate that both the chemical architecture and adhesive formulation play a significant role in the physical and mechanical properties of the adhesive films. Specifically, the hydrophobicity of the film had a significant impact on the extent of swelling, which was found to be inversely proportional to the mechanical properties and rate of hydrolysis. By designing the adhesive polymers with different compositions, the polymers were able to be tailored for these properties, which were further refined by blending these polymers with PCL-triol.

### Example 35

### Lap Shear Adhesion Strength of Adhesive Blends

The adhesive polymers Medhesive-096 or Medhesive-116 were coated on to bovine pericardium using the solvent casting method as described above. Solutions of the adhesive polymers were blended at the different concentrations and the mixture was applied to bovine pericardium as the backing material, and then allowed to dry slowly. Before forming the adhesive joint, a dilute solution of sodium periodate (NaIO₄, 20 mg/ml) was added to the pericardium substrate to oxidize the adhesives and lap shear testing was performed following ASTM F2255 protocols. Results for blends of Medhesive-096 and Medhesive-116 are shown in Table 12.

TABLE 12

| Weight % | Weight % | Maximum | | Work of |
|---|---|---|---|---|
| Medhesive- | Medhesive- | Adhesive | Strain at | Adhesion |
| 096 | 116 | Strength (kPa) | Failure | (J/m²) |
| 75 | 25 | 67.3 ± 29.4 | 0.53 ± 0.10 | 158 ± 124 |
| 66 | 33 | 31.5 ± 15.0 | 0.42 ± 0.13 | 74.6 ± 32.7 |
| 50 | 50 | 27.2 ± 12.6 | 0.36 ± 0.10 | 56.7 ± 35.5 |
| 33 | 66 | 13.8 ± 5.47 | 0.25 ± 0.14 | 20.6 ± 13.4 |

### Example 36

### Synthesis of 4-Arm-PEG-PLA-MA Block Copolymer

24.8 g of 4-arm PEG-OH (MW 2,000), 50.0 g of L-lactide (LA), and 200 mL of toluene was added to a round bottom flask equipped with a Dean-Stark apparatus and a condensation column. The mixture was heated in an oil bath (155-165° C.) until 100 mL of toluene was evaporated with argon purging. The mixture was allow to cool to room temperature before 643 μL of tin(II) 2-ethylhexanoate was added. The mixture was stirred in an oil bath (155-165° C.) with argon purging for overnight. Polymer was purified by precipitation in diethyl ether two times. The dried polymer was further reacted with triethylamine (15.1 mL) and methacrylate anhydride (17.4 mL) in 300 mL of chloroform for overnight. The polymer was purified with ether precipitation, followed by washing with 12 mM HCl, saturated NaCl solution, and water. After additional ether precipitation, 23 g of polymer was obtained. From 1H NMR (400 MHz, CDCl₃/TMS), number of LA repeat per arm is 21.1 and the overall MW of the polymer is 8,400 Da.

Blending with Amphiphilic Block Copolymer

Solutions of Medhesive polymers dissolved in either methanol or chloroform were blended with a solution of 4-arm PEG-PLA-MA block copolymer (combined polymer concentration of 100 mg/ml) and cast on to bovine pericardium as the backing material, and then allowed to dry slowly. Before forming the adhesive joint, a dilute solution aqueous of sodium periodate (NaIO₄, 20 mg/ml) was added to the pericardium substrate to oxidize the adhesives and lap shear testing was performed following ASTM F2255 protocols. Adhesive properties of adhesive blends are summarized in Tables 13 and 14 using bovine pericardium and bone tissue as the test substrates, respectively. Increased content of the block copolymer increased the adhesive properties.

TABLE 13

| Weight % | Maximum | | Work of |
|---|---|---|---|
| 4-arm | Adhesive | Strain at | Adhesion |
| PEG-PLA | Strength (kPa) | Failure | (J/m²) |
| 0 | 37.9 ± 11.5 | 0.42 ± 0.050 | 94.4 ± 42.2 |
| 5 | 101 ± 39.1 | 0.50 ± 0.10 | 173 ± 64.7 |
| 10 | 96.2 ± 58.6 | 0.48 ± 0.12 | 177 ± 74.5 |
| 20 | 137 ± 54.2 | 0.55 ± 0.060 | 267 ± 86.3 |

* Coated at 90 g/m²

TABLE 14

| Coating | Weight % | Maximum | | Work of |
|---|---|---|---|---|
| Density | 4-arm | Adhesive | Strain at | Adhesion |
| (g/m²) | PEG-PLA | Strength (kPa) | Failure | (J/m²) |
| 60 | 0 | 50.3 ± 15.9 | 0.53 ± 0.11 | 110 ± 21.0 |
| 60 | 20 | 62.6 ± 7.76 | 0.59 ± 0.18 | 121 ± 28.2 |
| 90 | 20 | 91.5 ± 18.4 | 0.40 ± 0.050 | 134 ± 32.1 |

### Example 37

### Multi-Layered Adhesive Coating

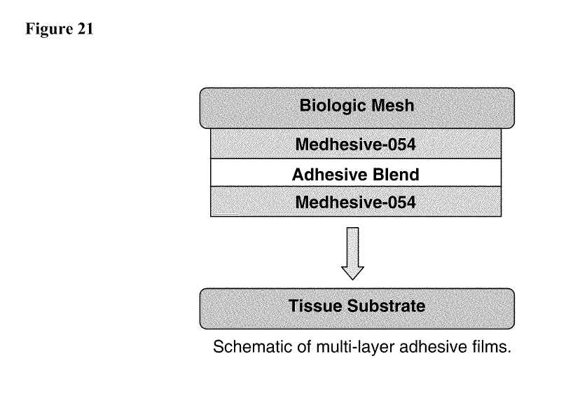

Multi-layer coating (FIG. 21) was achieved through successive solvent casting of Medhesive polymer solutions (dissolved in either methanol or chloroform) on to bovine pericardium as the backing followed by drying in vacuum. Lap shear adhesion tests (ASTM F2255) performed on trilayered adhesive coating is shown in Table 15 using bovine pericardium as the test substrate. The multilayer films consist of a 30 g/m² of Medhesive-112 (blended with 0-20 wt % with a 4-arm PEG-PLA-MA block copolymer) mid-layer sandwiched in between two 15 g/m² Medhesive-054 outer layers.

TABLE 15

| Weight % 4- | Maximum | | Work of |
|---|---|---|---|
| arm PEG-PLA | Adhesive | Strain at | Adhesion |
| in mid-layer | Strength (kPa) | Failure | (J/m²) |
| 0 | 184 ± 47.4 | 0.77 ± 0.28 | 499 ± 196 |
| 5 | 154 ± 42.7 | 0.73 ± 0.34 | 423 ± 95.5 |
| 20 | 190 ± 45.4 | 0.95 ± 0.21 | 576 ± 130 |

### Example 38

### Multi-Layered Adhesive Coating

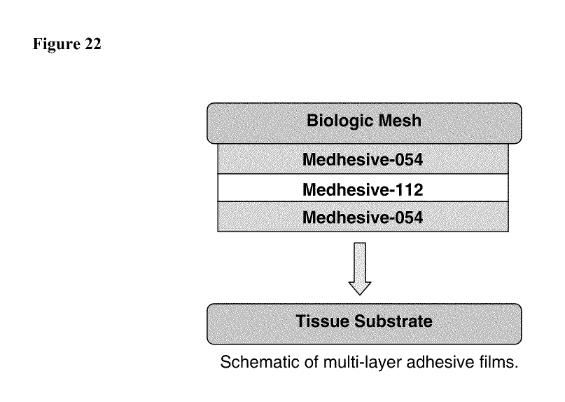

Multi-layer coating was achieved through successive solvent castings of Medhesive polymer solutions (dissolved in either methanol or chloroform) on to bovine pericardium followed by drying in vacuum. Lap shear adhesion tests (ASTM F2255) performed on trilayered adhesive coating is shown in Table 16 using bovine pericardium as the test substrate. Trilayer-1 consists of a 30 g/m² Medhesive-112 middle layer sandwiched in between two 15 g/m² Medhesive-054 outer layers while Trilayer-2 consists of a 60 g/m² Medhesive-112 middle layer sandwiched in between two 15 g/m² Medhesive-054 outer layers (See FIG. 22). These trilayered adhesives exhibited significantly improved adhesive properties as compared to a single layer of either Medhesive-054 or Medhesive-112. Performance of trilayer films on pieces of bone tissue cut from the scapula is shown in Table 17. Trilayer-3 consists of a 30 g/m² Medhesive-116 middle layer sandwiched in between two 15 g/m² Medhesive-054 outer layers while Trilayer-4 consists of a 60 g/m² Medhesive-116 middle layer sandwiched in between two 15 g/m² Medhesive-054 outer layers.

TABLE 16

| | Maximum | | |
|---|---|---|---|
| | Adhesive | | Work of |
| Adhesive | Strength | Strain at | Adhesion |
| Formulation | (kPa) | Failure | (J/m²) |
| Trilayer-1 | 185 ± 47.4 | 0.62 ± 0.19 | 499 ± 196 |
| Trilayer-2 | 144 ± 23.9 | 0.68 ± 0.19 | 400 ± 81.3 |
| Medhesive-054* | 39.0 ± 12.5 | 0.39 ± 0.070 | 71.6 ± 16.3 |
| Medhesive-112* | 8.48 ± 4.64 | 0.46 ± 0.26 | 18.6 ± 9.96 |

*Coated at 90 g/m²

TABLE 17

| | Maximum | | |
|---|---|---|---|
| | Adhesive | | Work of |
| Adhesive | Strength | Strain at | Adhesion |
| Formulation | (kPa) | Failure | (J/m²) |
| Trilayer-3 | 38.4 ± 21.4 | 0.34 ± 0.14 | 61.2 ± 44.1 |
| Trilayer-4 | 35.9 ± 14.1 | 0.52 ± 0.13 | 103 ± 53.0 |
| Medhesive-054* | 50.2 ± 15.9 | 0.53 ± 0.11 | 110 ± 21.0 |

*Coated at 60 g/m²

### Example 39

### Adhesive-Coated on Biotape™

A polymer solution of Medhesive (dissolved in either methanol or chloroform) was coated on a fluorinated release liner using the solvent casting method and dried with vacuum. The dried adhesive film was pressed against Biotape™ (Wright Medical Technology, Inc.), an acellular porcine matrix, and incubated at 55° C. for 1 hour. The bioadhesive construct was tested using lap shear adhesion test (ASTM F2255) using bovine pericardium as the test substrate. Maximum lap shear strength and work of adhesion were found to be 125±16.9 kPa and 269±64.6 J/m², respectively, for Medhesive-096 coated at 240 g/m². A trilayer adhesive coating consist of a 30 g/m² Medhesive-112 (blended with 20 wt % 4-arm PEG-PLA-MA) middle layer sandwiched in between two 15 g/m² Medhesive-054 outer layers demonstrated maximum lap shear strength and work of adhesion were found to be 79.3±9.18 kPa and 216±80.9 J/m², respectively.

### Example 40

### Tensile Testing of Adhesive Polymers

Medhesive polymers were cast into thin-films (70 mg/ml in chloroform) as described and their tensile mechanical properties were tested following ASTM standard D638 protocols. Tensile tests on dog-bone shaped films (9.53-mm gauge length, 3.80-mm gauge width, and 12.7-mm fillet radius, swollen in phosphate buffered saline (PBS) (pH 7.4) for 1 hr) were performed, and the maximal tensile strength was measured (Table 18). Both the Young's modulus and toughness were also determined, based on the initial slope and the area under the stress-strain curve, respectively.

TABLE 18

| | Young's | Maximum | | |
|---|---|---|---|---|
| Adhesive | Modulus | Strength | Strain at | Toughness |
| Formulation | (kPa) | (kPa) | Failure | (J/m²) |
| Medhesive-112 | 379 ± 53.9 | 449 ± 253 | 1.98 ± 1.31 | 716 ± 701 |
| Medhesive-116 | 479 ± 122 | 482 ± 122 | 1.40 ± 0.367 | 305 ± 111 |

### Example 40

### Synthesis of PCL1.25 k-diSA

10 g of polycaprolactone-diol (PCL-diol, MW=1,250, 8 mmol), 8 g of succinic anhydride (SA, 80 mmol), 6.4 mL of pyridine (80 mmol), and 100 mL of chloroform were added to a round bottom flask (250 mL). The solution was refluxed in a 75-85° C. oil bath with Ar purging for overnight. The reaction mixture was allowed to cool to room temperature and 100 mL of chloroform was added. The mixture was washed successively with 100 mL each of 12.1 mM HCl, saturated NaCl, and deionized water. The organic layer was dried over magnesium sulfate and then the volume of the mixture was reduced by half by rotary evaporator. After pouring the mixture into 800 mL of a 1:1 hexane and diethyl ether, the polymer was allowed to precipitate at 4° C. for overnight. The polymer was collected and dried under vacuum to yield 8.1 g of PCL1.25 k-diSA. NMR (400 MHz, DMSO/TMS): δ 12.2 (s, 1H, COOH—), 4.1 (s, 2H, PCL-CO—CH₂—CH₂—COOH—) 4.0 (s, 12H, O—(CO—CH₂—(CH₂)₄—O)₆CO—CH₂—CH₂—COOH), 3.6 (s, 2H, PCL-CO—CH₂—CH₂—COOH—) 3.3 (s, 21-1, —CH₂-PCL₆-SA), 2.3 (t, 12H, O—(CO—CH₂—(CH₂)₃—CH₂—O)₆CO—CH₂—CH₂—COOH), 1.5 (m, 24H, O—(CO—CH₂—CH₂—CH₂—CH₂—CH₂—O)₆CO—CH₂—CH₂—COOH), 1.3 (m, 12H, O—(CO—CH₂—CH₂—CH₂—CH₂—CH₂—O)₆CO—CH₂—CH₂—COOH). Similarly, PCL2k-diSA was synthesized using the procedure with 2,000 MW PCL-diol.

### Example 41

### Synthesis of PCL2k-diGly

10 g of polycaprolactone-diol (5 mmole, MW 2000) with 2.63 g of Boc-Gly-OH (15 mmole) was dissolved in 60 mL chloroform and purged with argon for 30 minutes. 3.10 g of DCC (15 mmole) and 61.1 mg of DMAP (0.5 mmole) were added to the reaction mixture and the reaction allowed to proceed overnight with argon purging. The solution was filtered into 400 mL of diethyl ether along with 40 mL of chloroform. The precipitate was collected through filtration and dried under vacuum to yield 4.30 g of PCL2k-di-BocGly. A Boc protecting group on PCL2k-di-BocGly was removed by reacting the polymer in 14.3 mL of chloroform and 14.3 mL of trifluoroacetic acid for 30 minutes. After precipitation twice in ethyl ether, the polymer was dried under vacuum to yield 3.13 g of PCL2k-diGly. ¹H NMR (400 MHz, CDCl₃/TMS): δ 4.2 (m, 4H, CH₂NH₂—) 4.0 (t, 16H, O—(CO—CH₂—(CH₂)₃CH₂—O)₈CO—CH₂—CH₂—COOH), 3.8 (t, 2H, O—CH₂CH₂—O—CO-PCL), 3.6 (t, 2H, O—CH₂CH₂—O—CO-PCL), 2.3 (t, 16H, O—CH₂CH₂—O—CO—CH₂(CH₂)₄—OCO), 1.7 (m, 32H, O—CH₂CH₂—O—CO—CH₂CH₂CH₂CH₂CH₂—OCO), 1.3 (m, 16H, O—CH₂CH₂—O—CO—CH₂CH₂CH₂CH₂CH₂—OCO). PCL1.25 k-diGly was synthesized using the similar procedure while using 1,250 MW PCL-diol.

### Example 42

### Synthesis of PEG10k-(SA)₄

100 g of 4-armed PEG-OH (10,000 MW; 40 mmol —OH), 20 g of succinic anhydride (200 mmol) was dissolved with 1 L chloroform in a round bottom flask equipped with a condensation column. 16 mL of pyridine was added and refluxed the mixture in a 75° C. oil bath with Argon purging overnight. The polymer solution was cooled to room temperature, and washed successively with equal volume of 12 mM HCl, nanopure water, and saturated NaCl solution. The organic layer was then dried over MgSO₄ and filtered. The polymer was precipitated from diethyl ether and the collected precipitate was dried under vacuum to yield 75 g PEG10k-(SA)₄. NMR (400 MHz, D₂O): δ 4.28 (s, 2H, PEG-CH₂—O—C(O)—CH₂—), 3.73-3.63 (m, PEG), 2.58 (s, 4H, PEG-CH₂—O—C(O)—C₂H₄—COOH). PEG10k-(GA)₄ was synthesized using the similar procedure while using glutaric anhydride instead of succinic anhydride.

### Example 43

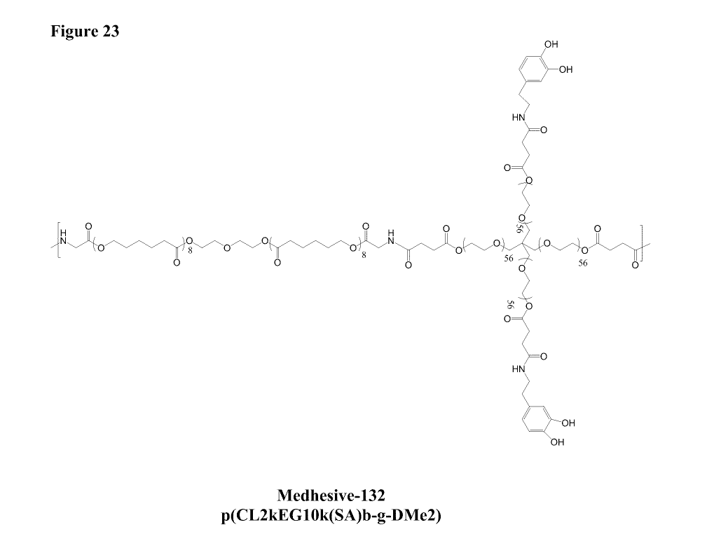

### Synthesis of Medhesive-132 (FIG. 23)

50 grams of PEG 10k-(SA)₄ was dissolved in 200 mL of DMF with 10.35 grams of PCL2k-diglycine, and 1.83 g of Dopamine-HCl in a round bottom flask. HOBt (3.24 g), HBTU (9.125 g), and Triethylamine (4.65 mL) was dissolved in 200 mL of chloroform and 300 mL of DMF. The HOBt/HBTU/Triethylamine solution was added dropwise to the PEG/PCL/Dopamine reaction over a period of 30-60 minutes. The reaction was stirred for 24 hours. 1.11 g of Dopamine and 1.01 mL Triethylamine was added to the reaction and stirred for 4 hours. The solution was filtered into diethyl ether and placed at 4° C. for 4-24 hours. The precipitate was vacuum filtrated and dried under vacuum for 4-24 hours. The polymer was dissolved in 400 mL of 50 mM HCl and 400 mL of methanol. This was then filtered using coarse filter paper and dialyzed in 10 L of water at pH 3.5 for 2 days with changing of the water at least 12 times. The solution was then freeze dried and placed under a vacuum for 4-24 hours. ¹H NMR (400 MHz, DMSO/TMS): δ 8.7-8.5 (s, 1H, C₆H₃(OH)₂—), 7.9 (d, 2H, C₆H₃(OH)₂—), 6.5 (dd, 1H, C₆H₃(OH)₂—), (dd, 1H, C₆H₃(OH)₂—CH₂—CH₂—CONH—CH₂—CH₂—O—), 4.1 (s, 2H, PCL-CO—CH₂—CH₂—COOH—), 4.0 (s, 16H, O—(CO—CH₂—(CH₂)₄—O)₆CO—CH₂—CH₂—COOH), 3.6 (s, 2H, PCL-CO—CH₂—CH₂—COOH—) 3.3 (s, 2H, —CH₂-PCL₆), 2.3 (t, 16H, O—(CO—CH₂—(CH₂)₃—CH₂—O)₆CO—CH₂—CH₂—COOH), 1.5 (m, 32H, O—(CO—CH₂—CH₂—CH₂—CH₂—CH₂—O)₆CO—CH₂—CH₂—COOH), 1.3 (m, 16H, O—(CO—CH₂—CH₂—CH₂—CH₂—CH₂—O)₆CO—CH₂—CH₂—COOH). UV-vis spectroscopy: 0.165±0.024 mmole Dopmaine/mg polymer (2.50±0.35 wt % Dopamine).

### Example 44

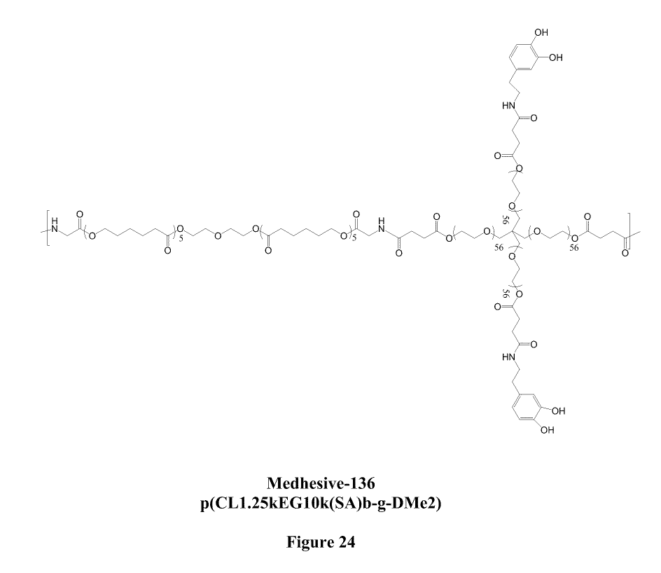

### Synthesis of Medhesive-0136 (FIG. 24)

20.02 grams of PEG10k-(SA)₄ was dissolved in 80 mL of DMF with 2.71 grams of PCL1.25k-diglycine, and 0.73 g of Dopamine-HCl in a round bottom flask. HOBt (1.30 g), HBTU (3.65 g), and Triethylamine (1.86 mL) was dissolved in 80 mL of chloroform and 120 mL of DMF. The HOBt/HBTU/Triethylamine solution was added dropwise to the PEG/PCL/Dopamine reaction over a period of 30-60 minutes. The reaction was stirred for 24 hours. 0.445 g of Dopamine and 0.403 mL Triethylamine were added to the reaction and stirred for 4 hours. The solution was filtered into diethyl ether and placed at 4° C. for 4-24 hours. The precipitate was vacuum filtered and dried under vacuum for 4-24 hours. The polymer was dissolved in 160 mL of 50 mM HCl and 160 mL of methanol. This was then filtered using coarse filter paper and dialyzed in 10 L of water at pH 3.5 for 2 days with changing of the water at least 12 times. The solution was then freeze dried and placed under a vacuum for 4-24 hours. After drying, ¹H NMR and UV-VIS were used to determine purity and coupling efficiency of the catechol. NMR (400 MHz, DMSO/TMS): δ 8.7-8.6 (s, 1H, C₆H₃(OH)₂—), 7.9 (d, 2H, C₆H₃(OH)₂—), 6.5-6.6 (dd, 1H, C₆H₃(OH)₂—), (dd, 1H, C₆H₃(OH)₂—CH₂—CH₂—CONH—CH₂—CH₂—O), 4.1 (s, 2H, PCL-CO—CH₂—CH₂—COOH—) 4.0 (s, 12H, O—(CO—CH₂—(CH₂)₄—O)₆CO—CH₂—CH₂—COOH), 3.6 (s, 2H, PCL-CO—CH₂—CH₂—COOH—) 3.3 (s, 2H, —CH₂-PCL₆-SA), 2.3 (t, 12H, O—(CO—CH₂—(CH₂)₃—CH₂—O)₆CO—CH₂—CH₂—COOH), 1.5 (m, 24H, O—(CO—CH₂—CH₂—CH₂—CH₂—CH₂—O)₆CO—CH₂—CH₂—COOH), 1.3 (m, 12H, O—(CO—CH₂—CH₂—CH₂—CH₂—CH₂—O)₆CO—CH₂—CH₂—COOH). UV-vis spectroscopy: 0.254±0.030 μmole Dopamine/mg polymer (3.86 f 0.45 wt % Dopamine).

### Example 45

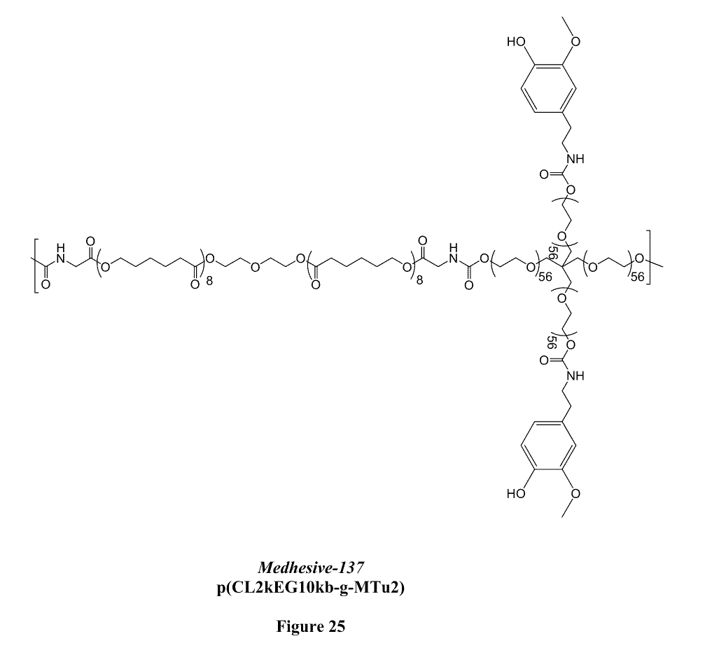

### Synthesis of Medhesive-137 (FIG. 25)

50 g of 10K, 4-arm PEG-OH (5 mmole) was combined with toluene (300 mL) in a 2000 mL round bottom flask equipped with a condenser, Dean-Stark Apparatus and Argon inlet. While purging with argon, the mixture was stirred in a 140-150° C. oil bath until 150 mL of toluene was removed. The reaction was cooled to room temperature and 53 mL (100 mmole) of 20% phosgene solution in toluene was added. The mixture was further stirred at 50-60° C. for 4 hours while purged with argon while using a 20 Wt % NaOH in a 50/50 water/methanol trap. Toluene was removed via rotary evaporation with a 20 Wt % NaOH solution in 50/50 water/methanol in the collection trap. The polymer was dried under vacuum for overnight. 3.46 g (30 mmole) of NHS and 375 mL of chloroform was added to PEG and the mixture was purged with argon for 30 minutes. 4.2 ml (30 mmole) of triethylamine in 50 mL chloroform was added dropwise and the reaction mixture was stirred with argon purging for 4 hours. After which, 2.3 g (11 mmole) of 3-methoxytyramine hydrochloride (MT) in 100 mL of DMF and 1.54 μl (11 mmole) of triethylamine was added and the mixture was stirred for 4 hours. 12 g (5 mmole) of PCL2k-diGly along with 800 mL of DMF was added followed by the addition of 1.4 mL of triethylamine to the mixture, which was further stirred for overnight. 0.72 g (3.5 mmole) of 3-methoxytyramine hydrochloride was added to cap the reaction along with 0.49 ml of triethylamine. The mixture was precipitated in 9 L of 50:50 ethyl diether and hexane, and the collected precipitated was dried under vacuum. The crude polymer was dissolved in 700 mL of methanol and dialyzed (15000 MWCO) in 10 L of water at pH 3.5 for 2 days. Lyophilization yielded the 45 g of Medhesive-137. ¹H NMR (400 MHz, DMSO/TMS): δ 8.7 (s, 1H, C₆H₃(OH)—), 7.6 (t, 1H, -PCL-O—CH₂—CH₂—NHCOO—CH₂—CH₂—O—)), 7.2 (t, 1H, —CH₂—CH₂—NHCOO—CH₂—CH₂—O—)), 6.7 (d, 1H, C₆H₃—), 6.6 (s, 1H, C₆H₃—), 6.5 (s, 1H, C₆H₃—), 4.1-4.0 (m, 32H, OOC(CH₂)₄CH₂—O), 3.8 (s, 3H, C₆H₃(OCH₃)), 3.8-3.3 (m, 224H, PEG), 3.1 (m, 2H, C₆H₃CH₂CH₂), 2.6 (t, 2H, C₆H₃CH₂CH₂), 2.3 (t, 32H, OOCCH₂(CH₂)₄—), 1.5 (m, 64H, —OOCCH₂CH₂CH₂CH₂CH₂—), 1.3 (m, 32H, OOCCH₂CH₂CH₂CH₂CH₂—). MT Wt %=2.97%; PCL Wt %=15.6%. UV-vis spectroscopy: 0.171±0.002 μmole MT/mg polymer (3.1±0.03 wt % MT).

### Example 46

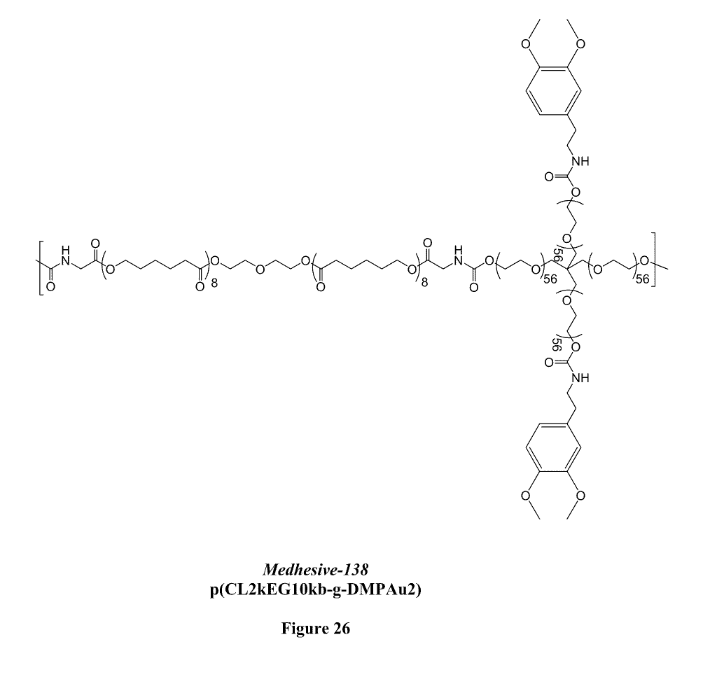

### Synthesis of Medhesive-138 (FIG. 26)

The procedure for synthesizing Medhesive-137 was used in the preparation of Medhesive-138 while using 3,4-dimethoxyphenylamine (DMPA) instead of 3-methoxytyramine hydrochloride. UV-vis spectroscopy: 0.215±0.005 μmole DMPA/mg polymer (3.9 f 0.09 wt % DMPA).

### Example 47

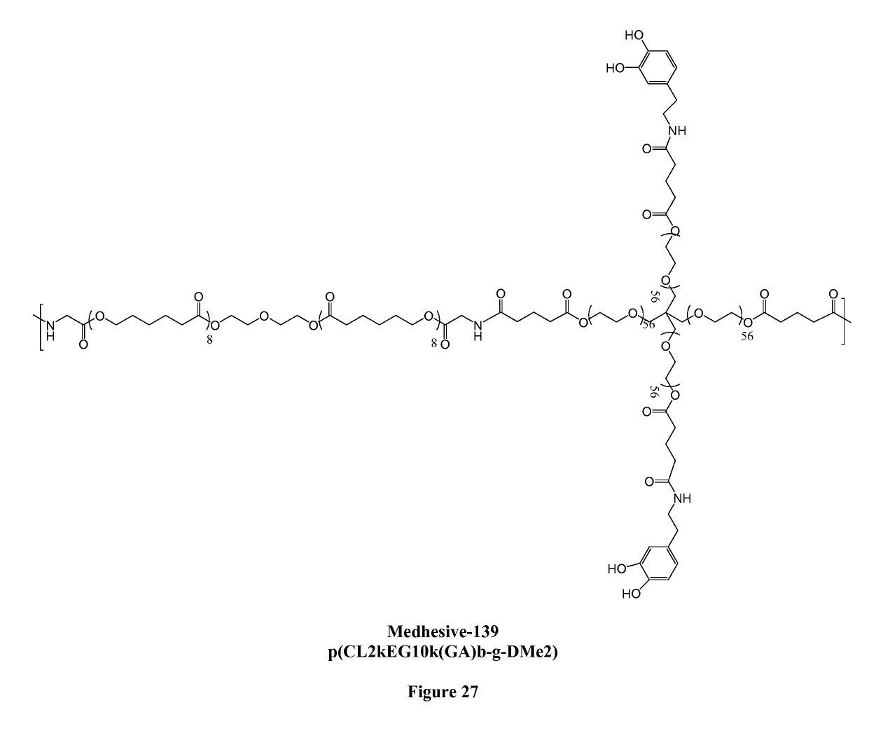

### Synthesis of Medhesive-139 (FIG. 27)

The procedure for Medhesive-132 was used in the synthesis of Medhesive-139 while using PEG10k-(GA)₄ instead of PEG10k-(SA)₄. ¹H NMR (400 MHz, DMSO/TMS): δ 8.7-8.6 (s, 1H, C₆H₃(OH)₂—), 7.9 (dd, 1H, C₆H₃(OH)₂—CH₂—CH₂—CONH—CH₂—CH₂—O—), 6.5-6.6 (dd, 1H, C₆H₃(OH)₂—), 4.1 (s, 2H, PCL-CO—CH₂—CH₂—COOH—) 4.0 (s, 16H, O—(CO—CH₂—(CH₂)₄—O)₈CO—CH₂—CH₂—COOH), 3.6 (s, 2H, PCL-CO—CH₂—CH₂—COOH—), 2.3 (t, 16H, O—(CO—CH₂—(CH₂)₃—CH₂—O)₈CO—CH₂—CH₂—COOH), 1.5 (m, 32H, O—(CO—CH₂—CH₂—CH₂—CH₂—CH₂—O)₈CO—CH₂—CH₂—COOH), 1.2-1.4 (m, 16H, O—(CO—CH₂—CH₂—CH₂—CH₂—CH₂—O)₈CO—CH₂—CH₂—COOH). UV-vis spectroscopy: 0.155±0.005 μmole Dopamine/mg polymer (2.36±0.08 wt % Dopamine).

### Example 48

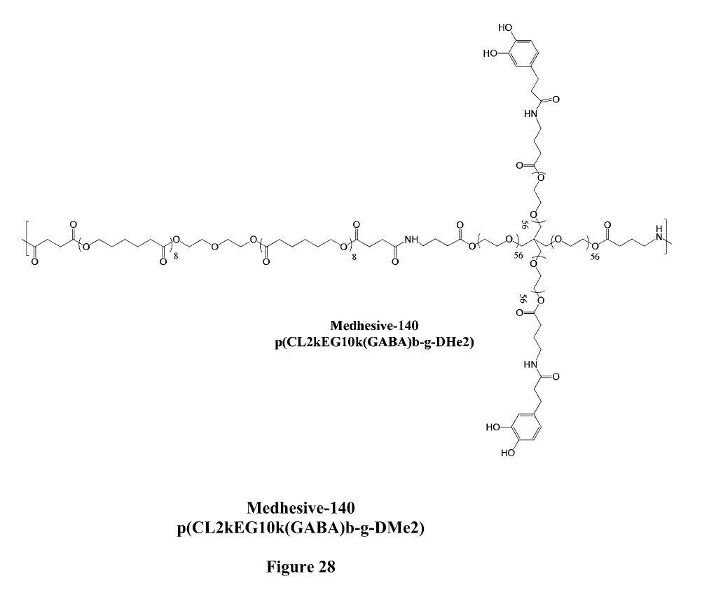

### Synthesis of Medhesive-140 (FIG. 28)

26.25 grams of PEG10k-(GABA)₄ was dissolved in 100 mL of DMF with 5.54 grams of PCL2k-diSA, and 1.14 g of DOHA in a round bottom flask. HBTU (4.74 g) and Triethylamine (2.42 mL) were dissolved in 100 mL of chloroform and 150 mL of DMF. The HBTU/Triethylamine solution was added dropwise to the PEG/PCL/DOHA reaction over a period of 30-60 minutes. The reaction was stirred for 24 hours. 0.69 g of DOHA and 0.525 mL Triethylamine were added to the reaction and stirred for 4 hours. This solution was filtered into diethyl ether and placed at 4° C. for 4-24 hours. The precipitate was vacuum filtrated and dried under vacuum for 4-24 hours. The polymer was dissolved in 400 mL of methanol. This was then filtered using coarse filter paper and dialyzed in 5 L of water at pH 3.5 for 2 days with changing of the water at least 12 times. The solution was then freeze dried and placed under a vacuum for 4-24 hours. After drying, ¹H NMR and UV-VIS were used to determine purity and coupling efficiency of the catechol. ¹H NMR (400 MHz, DMSO/TMS): δ 8.7-8.6 (s, 1H, C₆H₃(OH)₂—), 7.9 (dd, 1H, C₆H₃(OH)₂—CH₂—CH₂—CONH—CH₂—CH₂—O—), 6.5-6.6 (dd, 1H, C₆H₃(OH)₂—), 4.1 (s, 2H, PCL-CO—CH₂—CH₂—COOH—) 4.0 (s, 16H, O—(CO—CH₂—(CH₂)₄—O)₈CO—CH₂—CH₂—COOH), 3.6 (s, 2H, PCL-CO—CH₂—CH₂—COOH—), 2.3 (t, 16H, O—(CO—CH₂—(CH₂)₃—CH₂—O)₈CO—CH₂—CH₂—COOH), 1.5 (m, 32H, O—(CO—CH₂—CH₂—CH₂—CH₂—CH₂—O)₈CO—CH₂—CH₂—COOH), 1.2-1.4 (m, 16H, O—(CO—CH₂—CH₂—CH₂—CH₂—CH₂—O)₈CO—CH₂—CH₂—COOH). UV-vis spectroscopy: 0.237 f 0.023 μmole DOHA/mg polymer (39.1±0.38 wt % DOHA).

### Example 49

### Synthesis of PEG10k-(GABA)₄

150 g of PEG-OH (10,000 MW, 15 mmol) was combined with 300 mL of toluene in a 1 L round bottom flask equipped with a Dean-Stark apparatus, condensation column, and an Argon inlet. The mixture was stirred at 160° C. in an oil bath with Argon purging until 70-80% of the toluene had been evaporated and collected. The reaction mixture was cooled to room temperature. 350 mL of chloroform was added along with 36.6 g (180 mmol) of N-Boc-gamma-aminobutyric acid (Boc-GABA-OH) in 325 mL of chloroform were added to the reaction mixture. 37.1 g (180 mmol) of DCC and 733 mg (6 mmol) of DMAP was added to the reaction mixture. The reaction was stirred under Argon for overnight. The insoluble urea was filtered through vacuum filtration and the resulting mixture was filtered into 3.75 L of ether and the precipitate was collected through vacuum filtration and dried under vacuum for 22 hours. A total of 145.5 g of material was collected and was dissolved in 290 mL of chloroform. 290 mL of trifluoroacetic acid was added slowly to the reaction mixture and was allowed to stir for 30 minutes. The polymer solution was reduced to half through rotary evaporation. The solution was then added to 3 L of ether and placed at 3-5 C for 20 hours. The precipitate was dried under vacuum for 48 hours. A total of 156 g of material was obtained and dissolved in 1560 mL of nanopure water. The solution was then suction filtered and dialyzed (2000 MWCO) against 10 L of nanopure water for 4 hours followed by acidified water (pH 3.5) for 44 hours. The solution was then dialyzed against nanopure water for 4 hours. The solution was filtered lyophilized to yield 83.5 g of PEG10k-(GABA)₄. ¹H NMR (400 MHz, D₂O): δ 4.2 (m, 2H, PEG-CH₂—OC(O)—CH₂—), 3.8-3.4 (m, 224H, PEG), 3.0 (t, 2H, PEG-OC(O)—CH₂CH₂CH₂—NH₂), 2.5 (t, 2H, PEG-OC(O)—CH₂CH₂CH₂—NH₂), 1.9 (t, 2H, PEG-OOC—CH₂CH₂CH₂—NH₂).

### Example 50

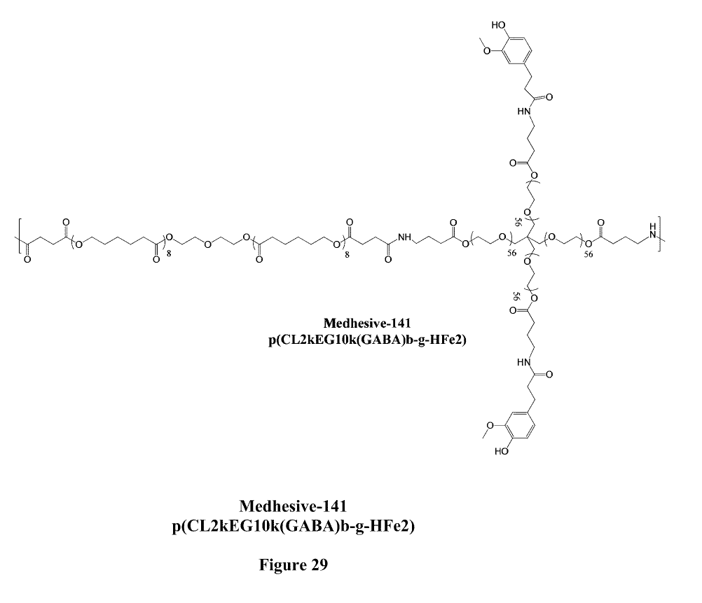

### Synthesis of Medhesive-141 (FIG. 29)

26.22 g (2.5 mmol) of PEG10k-(GABA)₄, 5.5 g (2.5 mmol) of PCL2k-diSA, and 1.228 g (6.25 mmol) of hydroferulic acid (HF) was dissolved in 100 mL of DMF. 4.74 g (12.5 mmol) of HBTU and 2.42 mL of triethylamine (17.4 mmol) was dissolved in 150 mL of DMF and 100 mL of chloroform. The HBTU and triethylamine solution was added to an addition funnel and was added dropwise to the PEG10k-(GABA)₄, PCL2k-diSA, and hydroferulic acid solution over a period of 40 minutes. The reaction was stirred at room temperature for 24 hours. 747 mg (3.8 mmol) of hydroferulic acid was added to the reaction along with 0.525 mL (3.77 mmol) of triethylamine. The reaction was allowed to stir an additional 4 hours. The reaction was gravity filtered into 2.2 L of a 1:1 ditheyl ether/hexane mix. The solution was then placed at 4° C. for 18 hours. The precipitate was suction filtered and dried under vacuum for 48 hours. The precipitate was then dissolved in 400 mL of methanol and placed in 15000 MWCO dialysis tubing. The mixture was dialyzed against 5 L of acidified nanopure water for 44 hours with changing of the dialysate 10 times. The solution was then dialyzed against 5 L of nanopure water for 4 hours with changing of the solution 4 times. The solution was suction filtered, frozen in a lyophilizer flask, and freeze dried. 27.3 g of Medhesive-141 were obtained. ¹H NMR (400 MHz, DMSO/TMS): δ 8.6 (s, 1H, C₆H₃(OH)—), 7.9 (t, 1H, -PCL-O—CH₂—CH₂—NHCO—CH₂—CH₂—O—)), 7.8 (t, 1H, —CH₂—CH₂—NHCO—CH₂—CH₂—O—)), 6.7 (d, 1H, C₆H₃—), 6.6 (s, 1H, C₆H₃—), 6.5 (s, 1H, C₆H₃—), 4.1 (m, 2H, PEG-CH₂—OOC-GABA), 4.0 (m, 2H, PEG-CH₂—OOC-GABA), 3.9 (m, 2H, O—CH₂(CH₂)₄—COO—), 3.7 (s, 3H, C₆H₃(OCH₃) 3.4 (m, 224H, PEG), 3.0 (t, 2H, PEG—OC(O)—CH₂CH₂CH₂—NH₂), 2.7 (t, 2H, C₆H₃CH₂CH₂), 2.5 (t, 2H, PEG-OC(O)—CH₂CH₂CH₂—NH₂), 2.3 (m, 4H, NHOC—CH₂CH₂COO-PCL), 2.3 (m, 32H, —(CH₂)₄—CH₂COO—), 1.6 (m, 2H, PEG-OOC—CH₂CH₂CH₂NH—), 1.6 (m, 64H, —CH₂CH₂CH₂CH₂CH₂COO—), 1.3 (m, 32H, CH₂CH₂CH₂CH₂CH₂COO—): HF Wt %=2.63%; PCL Wt %=17.5%. UV-vis spectroscopy: 0.156±0.011 μmole HF/mg polymer.

### Example 51

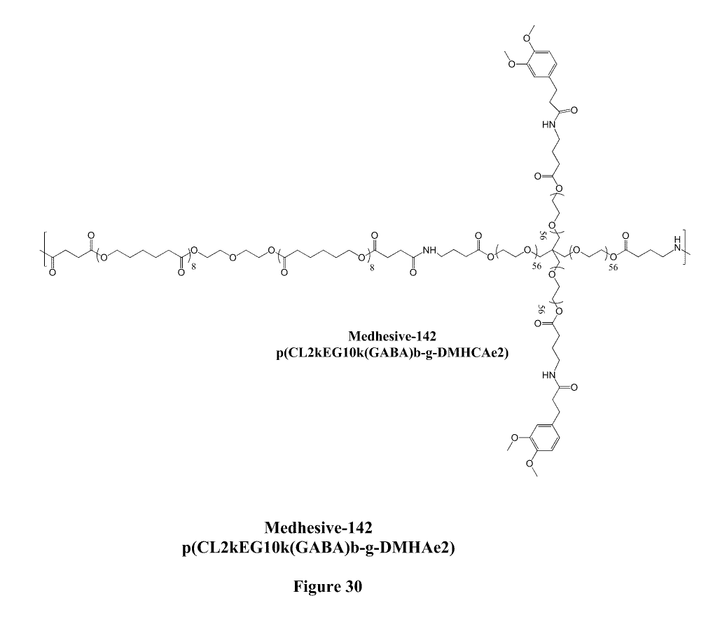

### Synthesis of Medhesive-142 (FIG. 30)

The same procedure for Medhesive-141 was used except instead of hydroferulic acid, 3,4-dimethoxyhydrocinnamic acid (DMHA) was used. UV-vis spectroscopy: 0.180±0.007 μmole DMHA/mg polymer.

### Example 52

### Method for Coating Adhesive onto Mesh Using Solvent Casting

The adhesive polymers were dissolved at 5-15 wt % in chloroform, methanol, or mixture of these solvents. The polymer solutions were solvent casted over the mesh, which is sandwiched between a PTFE mold (80 mm×40 mm or 80 mm×25 mm) and a release liner. The PTFE is sealed with double sided tape or PTFE films with the same dimension as the mold. Typical polymer coating density is between 60 and 240 g/m². The solvent was evaporated in air for 30-120 minutes and further dried with vacuum.

### Example 53

### Method for Preparing Stand-Alone Thin-Film

A stand alone film was made by solvent casting a polymer solution onto a release liner with a PTFE mold using similar parameters and conditions as the solvent casting method. The solvent was evaporated in air for 30-120 minutes and further dried with vacuum.

### Example 54

### Method for Coating Adhesive onto Mesh Using Heat-Press

A stand-alone thin-film adhesive was pressed against a mesh in between two glass plates using clamps. The samples were placed in an oven (55° C.) for 20-120 minutes to yield the adhesive-coated mesh.

### Example 55

### Method for Preparing Oxidant Embedded Stand-Alone Thin-Film

A stand-alone thin-film was made by solvent casting a non-reactive polymer (i.e. Medhesive-138) solution with oxidant onto a release liner with a PTFE mold using similar parameters and conditions as the solvent casting method. The solvent was evaporated at 37° C. for 30-120 minutes and dried under vacuum.

### Example 56

### Method for Preparing Adhesive-Coated Mesh Embedded with Oxidant

An oxidant embedded stand-alone thin-film was heat pressed over a mesh coated with adhesive in between two clamped glass plates. The samples are placed in the oven at 55° C. for 10-60 minutes and placed in the freezer for 5-30 minutes. The samples are then dried under vacuum.

### Example 57

### Method for Lap Shear Adhesion Testing

Lap shear adhesion tests was performed following ASTM procedures (ASTM F2392). Both the adhesive coated-mesh and the test substrates were cut into 2.5 cm×3 cm strips unless stated otherwise. The adhesive was activated through spraying of 20 mg/mL solution of NaIO₄ (PBS was added to NaIO₄ embedded samples) prior to bringing the adhesive into contact with the test substrate. The adhesive joint was compressed with a 100 g weight for 10 min, and further conditioned in PBS (37° C.) for another hour prior to testing. The adhesives were pulled to failure at 10 mm/min using a universal tester.

### Example 58

### Method for In Vitro Degradation

Adhesive coated meshes are cured using 20 mg/mL NaIO₄ solution and then incubated in PBS (pH 7.4) at either 37 or 55° C. At a given time point, the samples are dried with vacuum and weighed. The mass loss overtime is then reported.

### Example 59

### Degradation profile of Medhesive-132

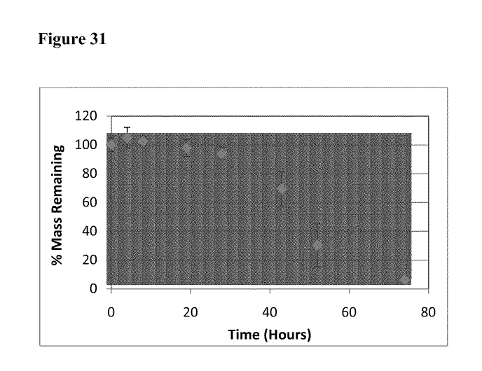

Medhesive-132 coated on a PE mesh was completely degraded with 3-4 days of incubation in PBS (pH 7.4) at 37° C. (FIG. 31). When incubated at a higher temperature (55° C.), Medhesive-132 films completely dissolve within 24 hours. Although Medhesive-132 has a similar PCL content (−20 wt %) as Medhesive-096, Medhesive-096 lost only 12% of its original mass over 120 days. This indicates that hydrolysis occurs at a faster rate for the ester bond linking PEG and succinic acid than those within the PCL block. PEG is more hydrophilic than PCL and increased water uptake resulted in faster degradation rate.

### Example 60

### Performance of adhesive-coated on PTFE mesh

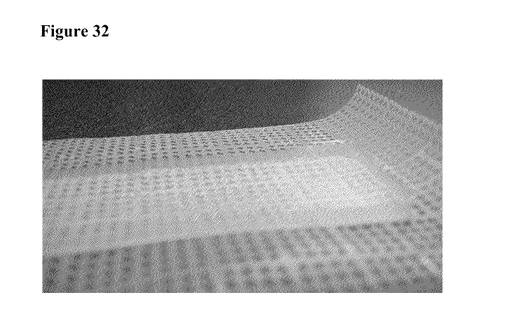

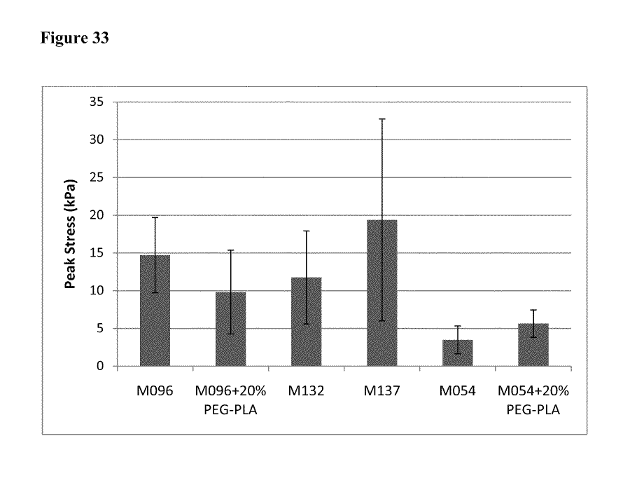

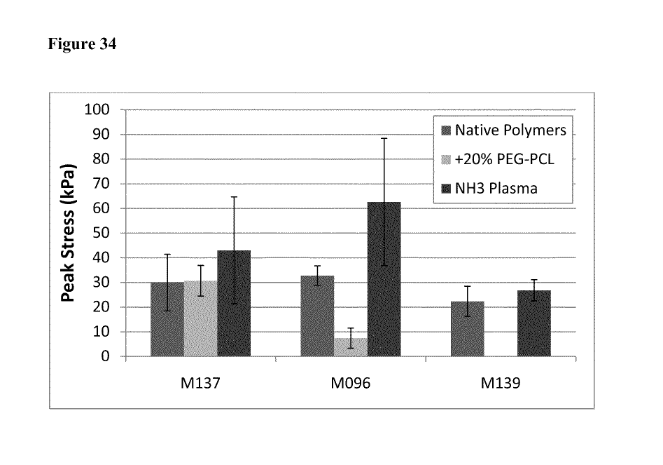

Several adhesive formulations were coated onto PTFE (Motif) mesh using solvent casting method (FIG. 32) and lap shear adhesion test was performed (FIG. 33, FIG. 34) Adhesive formulations were blended with either 4-armed PEG-PLA or PEG-PCL up to 20 wt %. PTFE treated with ammonium plasma for 3 min prior to coating in resulted in higher peak stress value for Medhesive-096.

### Example 61

### Performance of Adhesive-Coated on Polyester Mesh

Various adhesives were solvent casted on to PETKM2002 polyester (PE) mesh (0.5 mm pore, 30 g/m²) and lap shear adhesion test was performed (Table 19). These adhesives all demonstrated strong water-resistant adhesive properties to bovine pericardium. The maximum shear strengths measured were between 56 and 78 kPa.

TABLE 19

| | Maximum Strength (pKa) | | |
|---|---|---|---|
| | | | Number |
| | Average | St. Dev. | of repeat |
| Medhesive-139 | 56.2 | 20.9 | 30 |
| Medhesive-140 | 77.7 | 25.9 | 17 |
| Medhesive-141 | 57.4 | 27.3 | 12 |

*240 g/m² coating density

### Example 62

### Performance of Adhesive-Coated on Polypropylene Mesh

Stand-alone thin-film adhesives were heat-pressed onto NovaSilk polypropylene (PP) mesh at a coating density of 240 g/m² and lap shear adhesion test was performed (Table 20). Medhesive-096 formulations generally fail at the adhesive-tissue interface. On the other hand, Medhesive-054+20 wt % PEG-PLA demonstrate a maximum load of 5.5±0.8 pounds of force prior to complete rupture of the adhesive joint. In most cases, this formulation resulted in failure of the synthetic mesh material prior to failure for the adhesive.

TABLE 20

| | PEG- | Maximum Load | | Maximum | |
|---|---|---|---|---|---|
| | PLA | (Lbf) | | Strength (pKa) | |
| Adhesive Type | (wt %) | Average | St. Dev. | Average | St. Dev. |
| Medhesive-054 | 0 | 3.3 | 0.6 | 12 | 2.0 |
| Medhesive-054 | 20 | 5.5 | 0.8 | 19 | 3.0 |
| Medhesive-096 | 0 | 3.5 | 0.7 | 12 | 2.2 |
| Medhesive-096 | 20 | 2.2 | 0.7 | 7.5 | 2.5 |

*240 g/m² coating density; contact area = 500-600 mm²; pulled at 5 mm/min.

### Example 63

### Performance of Oxidant-Embedded PE Mesh

Oxidant embedded films were tested for adhesion using PETKM2002 PE mesh (Table 21). The adhesive films were coated with 240 g/m² of adhesive film on one side of PE mesh and 120 g/m² of none-reactive film on the other side, which is embedded with NaIO₄. The formulations were activated by applying moisture (PBS) to both sides of the mesh while in contact with tissue.

TABLE 21

Maximum
Strength (pKa)

| | | | St. |
|---|---|---|---|
| Adhesive Layer | Non-reactive Layer | Average | Dev. |
| Medhesive-137 | Medhesive-138 | 88.0 | 32.2 |
| Medhesive-141 | Medhesive-142 | 104 | 26.4 |

### Example 64

### Performance of adhesive-coated on human dermis

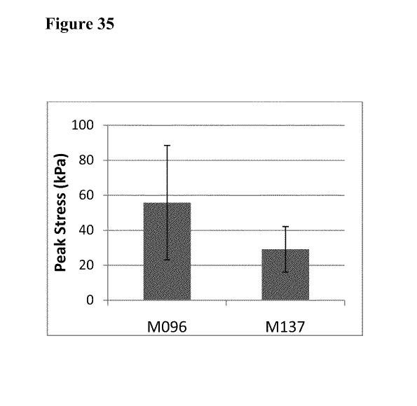

Adhesives were formulated into stand alone thin-films at a coating density of 150 g/m² and heat pressed onto human dermis for 1 hr at 55° C. Lap shear adhesion test was performed and peak stress was determined (FIG. 35).

### Example 64

### Adhesion of Adhesive Construct to Bone

Medhesive-137 was coated on bovine pericardium at 90 g/m² and the effect of adhesive joint incubation prior to testing was determined (FIG. 36). Adhesives were also blended with up to 20 wt % of 4-arm PEG-PLA or PEG-PCL. The sample was either unmodified (no hydration), covered in moist gauze to keep the adhesive joint wet, or soaked in saline prior to testing. Medhesive-137 with no additional hydration showed the highest adhesive strength to bone of all samples tested.

### Example 65

### Adhesive-Coated Construct in Rotator Cuff Repair

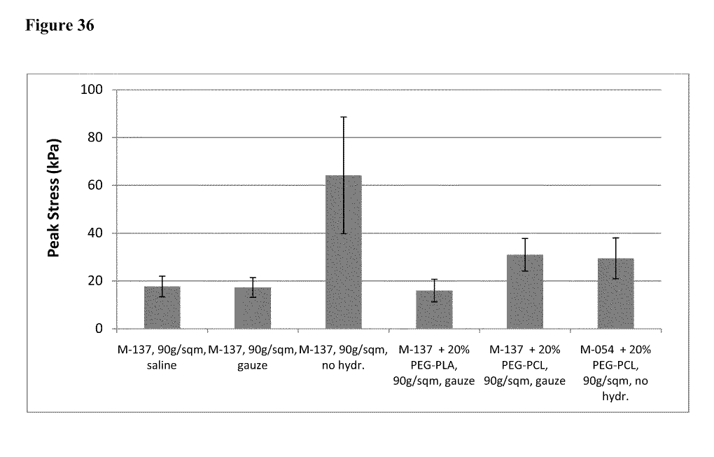

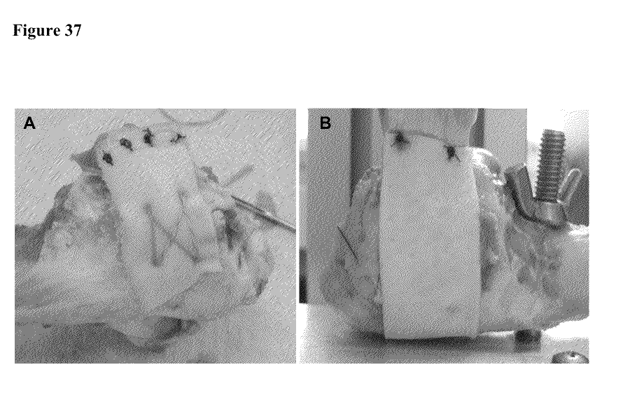

Adhesive-coated Biotape was used to augment primary suture repaired ovine shoulder and compared to non-adhesive Biotape which was secured using sutures. (FIG. 37) In both test groups, ovine shoulders were first repaired with primary suture repair. Briefly, the infraspinatus tendon was completely released from its attached point using a scalpel and the tendon was secured using a double-row fixation. Suture anchors (Arthrex 5.5 mm Bio-Corkscrew) were placed medial to the tendon footprint and sutures were tied through the distal end of the tendon using a mattress stitch. The suture tails were then passed across the lateral border of the tendon and inserted through transosseous tunnels to form the lateral row. The primary repair was further augmented with Biotape (approx. 20×60 mm) which was anchored to the musculotendinous junction using four interrupted absorbable sutures. The suture tails were passed up through the Biotape above the mattress stitches and then down through the transosseous tunnels. For adhesive-coated (Medhesive-137+20 wt % PEG-PCL) Biotape augmented repair, the construct was first anchored to the musculotendinous junction using two interrupted absorbable sutures. The adhesive film was then activated by spraying with a mist of aqueous crosslinking solution (NaIO₄, 20 mg/mL). The film was immediately approximated to the tissue surface and was covered with moist gauze. The repaired tissue assembly was incubated at 37° C. for 1 h prior to mechanical testing.

Mechanical testing was performed on computer-controlled servomechanical universal testing machine (ADMET, Inc., Norwood, Mass.) equipped with a 500N load cell. Repaired tendon-humerus assemblies were placed in a custom-fabricated bracket which was affixed in the test grips. The repaired tendons were preloaded cyclically from 2-10 N for 10 cycles to permit alignment of the tendon fibers and subsequently pulled to failure at 10 mm/min.

Mechanical testing result demonstrated no significant differences between shoulders augmented with non-adhesive and adhesive-coated Biotape (Table 22). These data suggest that a repair that is as strong as sutured Biotape can be achieved in less time since fewer sutures are used in conjunction with the Medhesive-coated Biotape. The resultant reduction in repair time would be expected to reduce overall operative time and associated costs.

TABLE 22

| | Sutured Biotape | Biotape + M-137 | |
|---|---|---|---|
| | (n = 5) | (n = 9) | Definition |
| Relative Stiffness | 18.9 ± 6.8 | 21.2 ± 4.0 | Based on the |
| (N/mm) | | | slope prior to |
| | | | failure |
| Tendon Peak | 192 ± 86.4 | 228 ± 75.0 | Maximum load |
| Load (N) | | | prior to rupture |
| | | | of the tendon |
| Medhesive | N/A | 191 ± 62.5 | Maximum load |
| Failure Load (N) | | | prior to |
| | | | detachment of |
| | | | BioTape from |
| | | | the humerus |

### Example 66

### Cytoxicity

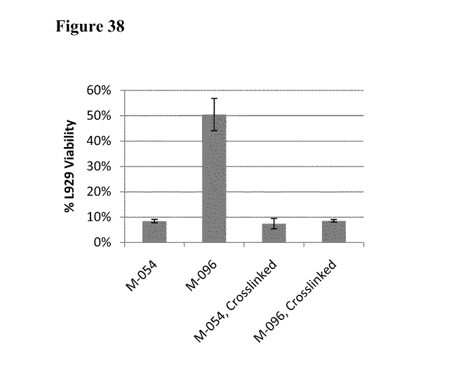

Cytotoxicity testing was performed on adhesive formulations using the MEM Elution assay according to ISO 10993-5. In activated adhesive formulations (activated with an oxidant, NaIO4) were placed in 50 mL culture tubes and covered with MEM growth media. Test articles were extracted for 24 h at 37° C., and L-929 fibroblasts were incubated in these extracts for 48 hours at 37° C. Cell viability was then determined using the MIT assay, with 80% cell viability needed to pass the cytotoxicity test. Certain adhesive formulations (Medhesive-054 and -096) may be cytotoxic (FIG. 38).

To determine the source of cytotoxicity, a series of polyethylene glycol (PEG) modified with adhesive catechol moieties (dopamine or 3,4-hydroxyhydrocinnamic acid) as model compounds were used. The PEG-catechol conjugates have similar compositions (˜80-87% similarity), starting materials (PEG and catechol), and synthesis reagents as the adhesives used in the current project, while at the same time having a reduced number of synthesis steps and simpler characterization methodologies. For example, the PEG-catechol conjugates have fixed molecular weights and are water soluble. Additionally, cytotoxicity of each key component (i.e., catechol and PEG) used to synthesized the adhesive polymers was determined. It was found that during the process of oxidation, either mediated by the crosslinker or through auto-oxidation of catechol, reactive oxygen species (ROS) were produced in the cell culture media, which contributed to high oxidative stress and a “pro-oxidant” environment which led to cell death. Furthermore, in vitro growth media are generally deficient in the protective mechanisms present in vivo, rarely containing antioxidants like ascorbic acid or tocopherol. When antioxidants (ascorbic acid) or free radical scavengers (superoxide dismutase, catalase, and glutathione) were formulated into the media, an increase in cell viability was observed.

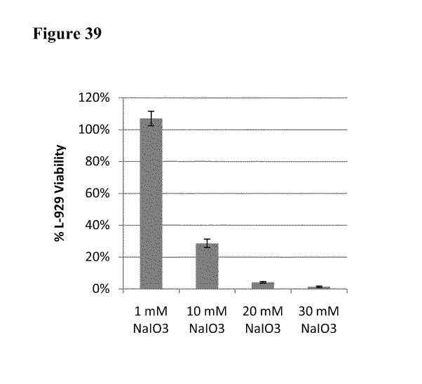

Inherent cytotoxicity is one of the suspected byproducts of the crosslinking reagent (NaIO4) used. NaIO4 is necessary for transforming catechol into highly reactive quinone, which can react with a tissue surface through covalent bond formation. During the crosslinking process, sodium periodate is reduced to sodium iodate (NaIO3), which in turn is further reduced to sodium iodide. A dose response of sodium iodate (NaIO3) in ISO Elution testing was performed, and NaIO3 was found to be cytotoxic at quantities greater than 1-10 mM (FIG. 39).

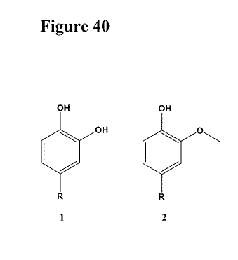

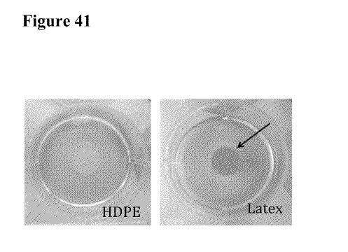

To further improve the biocompatibility of our adhesive, we have synthesized polymers functionalized with a methoxy group at the meta-position (FIG. 40, compound 2) instead of a dihydroxy catechol (FIG. 40, compound 1). These adhesive moieties are unable to undergo auto-oxidation and have been shown to be non-cytotoxic in our MEM eluting assay. We developed the capability to conduct cytotoxicity assays by the agarose overlay method (FIG. 41) ISO 10993-5), which has been frequently used for other commercial tissue adhesives (FLOSEAL™, COSEAL™, ProGEL™, and DuraSeal®). Per the guidelines provided by ISO 10993-5, we conclude that this methodology is appropriate for the proposed product configuration of the thin-film adhesive applied to a surgical mesh. We will use this method in screen our adhesive formulations as well as oxidant type and oxidant delivery method and preliminary animal studies will be designed to screen adhesive formulations.

### Example 67

### Mesh Types

Biologic mesh acts as an extracellular matrix that is closer in composition to native tissue, and can degrade and be remodeled in vivo. Additionally, biologic meshes have reduced the rate of postoperative infection, and can be used for treatment in an infected field. However, synthetic meshes are used more frequently in the clinical setting than their biologic counterparts, are more developed, and their performance has been better documented. Modifications to synthetic meshes may be more easily adopted by surgeons. Cost of materials is also a concern as synthetic meshes are a couple of orders of magnitude less expensive than biologic meshes. Mesh materials include: expanded and condensed polytetrafluoroethylene (PTFE), polyester (PE), polypropylene (PP) of varying weights and pore sizes, polypropylene-based composite meshes with a variety of absorbable and nonabsorbable adhesion barriers, polyester-based composite meshes with adhesion barriers, and resorbable meshes (polylactic and polyglycolic acid). Polypropylene meshes or composite meshes with a polypropylene base and resorbable anti-adhesion barrier are the most widely used. However, it has been reported that available PP meshes are over-engineered, having stiffness that is 10 time stronger than the abdominal wall. Additionally, heavy-weight PP meshes with small pore size leads to an intense inflammatory reaction that results in rapid incorporation into the abdominal wall. The fibrosis spans the small space between the threads, forming a dense scar plate that encapsulates the entire mesh, reducing abdominal compliance. Polypropylene also leads to formation of tenacious adhesions. The scar plate is formed to a lesser extent in lighter-weight meshes with larger pores. The fibrosis surrounds each fiber, but is not connected, and doesn't form as rigid of a scar plate with as much mesh shrinkage as heavy-weight polypropylene meshes. Delayed or less robust ingrowth can actually be better in that it may more closely match the compliance of the native abdominal wall. Reduction in scar plate formation seems to correlate with reduction in polypropylene material. Because of its widespread use, and greater flexibility and lower inflammatory response compared with heavy-weight polypropylene mesh, light-weight polypropylene mesh with large pore size is included as a mesh type. Polyester, when used alone or in combination with an adhesion barrier, generally exhibits less inflammatory response, fewer adhesions, and better incorporation than PP. PE is chosen as a second mesh type for further consideration with our technology. Additionally, both raw PP and PE meshes are available in large quantities by multiple vendors, which make them ideal for development work. Ciniclan reports of PTFE meshes are positive. Thin PTFE with large sized pores exhibits better or equivalent inflammatory response, scar plate formation, and integration with abdominal wall tissues compared with PP composite meshes. It can also be visualized with current imaging techniques. PTFE is used as an alternative backing if difficulty is encountered working with either PP or PP meshes. Degradable meshes may also be used.

### Example 68

### Synthesis of New Polymers

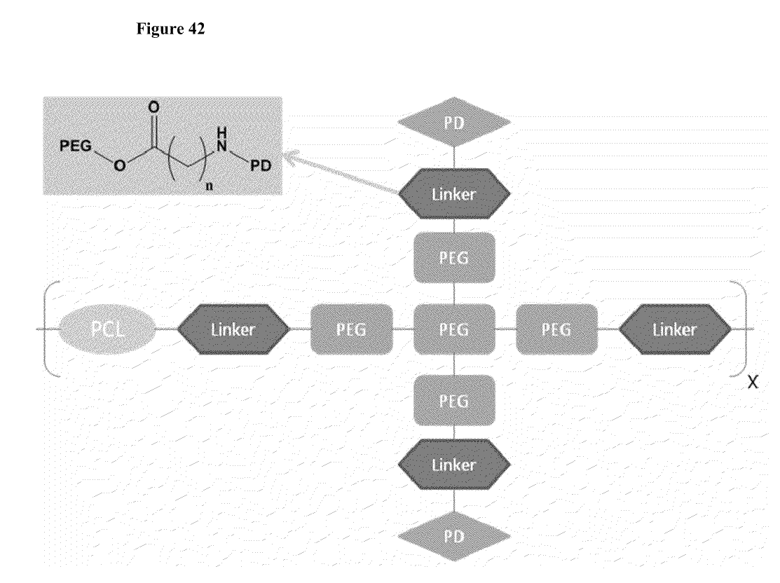

Early adhesive polymers have acceptable adhesive properties, however their degradation rates were on the order of months to possibly years. Given that tissue-mesh integration is nearly complete after 2-4 weeks, it is necessary to tailor adhesives to degrade within months so that the adhesive does not act as a barrier for tissue ingrowth when its function is no longer needed. To increase the degradation rate, a modification in the chemical architecture was made, where a hydrolysable ester linkage is inserted between the hydrophilic PEG and adhesive molecule, DHP (FIG. 42). For example, Medhesive-132, with a succinic acid linker that forms an ester bond with PEG, degrades in less than 24 hours under accelerated conditions (55° C.) in vitro. The hydrophobicity of the linker is adjusted to further fine tune the rate of degradation.

In addition to modulating the rate of degradation, a polymer (Medhesive-137) with a more biocompatible adhesive moiety, 3-methoxy, 4-hydroxyphenyl(FIG. 40, compound 2) was synthesized. The 3-methoxy group does not undergo auto-oxidation, and will not generate ROS that contributed to cell death in in vitro cell culture.

### Example 69

### Adhesive-Coating on Synthetic Mesh

Polymer solutions in either chloroform or methanol were solvent casted onto the mesh at different coating densities (90-240 g/m2). Additionally, both PP and PE meshes of different mesh weights and pore sizes were used, and lap shear adhesion tests were performed. The adhesive results were favorable as these adhesive-coated meshes and demonstrated strong adhesive properties to wetted tissue (bovine pericardium) and reproducibility (Table 23). While these values are slightly lower than those obtained using biologic meshes (50-100 kPa), adhesive formulations are optimized to improve the performance of adhesive-coated on synthetic meshes.

TABLE 23

| Adhesive | | | Mesh Weight | | | | Lap Shear |
|---|---|---|---|---|---|---|---|
| Formulation* | | Mesh Type | (g/m₂) | | Pore Size (mm) | | Strength |
| Average (kPa) | | St. Dev. (kPa) | | CV** | | Sample Size | |
| Medhesive-132 | PP | 25 | 1.5 × 1.2 | 39.0 | 14.1 | 36.3 | 28 |
| Medhesive-132 | PP | 68 | 1.0 | 36.6 | 12.4 | 33.8 | 12 |
| Medhesive-132 | PE | 30 | 0.5 | 39.7 | 13.9 | 35.0 | 30 |
| Medhesive-139 | PE | 30 | 0.5 | 56.2 | 20.9 | 37.1 | 30 |

*Coating density of 240 g/m2
**Coefficient of Variation; CV = St. Dev./Average × 100

### Example 70

### Oxidant Embedding

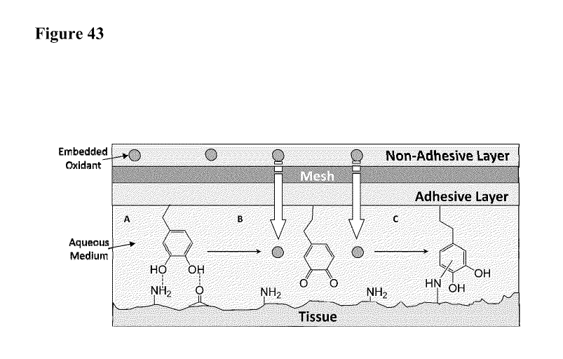

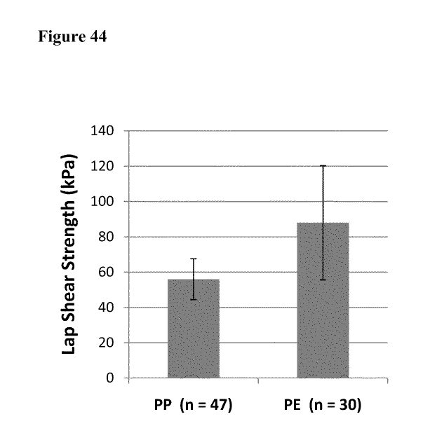

The adhesive is activated by spraying an oxidant solution (NaIO4) onto the adhesive film prior to contacting tissue. While strong adhesive strength was demonstrated, this oxidant delivery method may not be desirable in the clinical setting, and excess oxidant may cause irritation. Specifically, the oxidants may be cytotoxic. To simplify the delivery of oxidant and enhance general biocompatibility of the adhesive films, we have developed a method to embed the oxidant using a multi-layer approach (FIG. 43). The oxidant is embedded in a non-reactive polymer (Non-Adhesive Layer, Medhesive-138) and then heat pressed over the top of an adhesive-coated mesh. To activate the adhesive, an aqueous solution is added to the films. As the films swell, the oxidant is dissolved and diffuses into the adhesive layer (Medhesive-137) in contact with tissue, which results in formation of an interfacial bond. A controlled amount of oxidant is delivered to the adhesive film and reduced to its benign form prior to contact with the abdominal wall. This method has shown excellent adhesive performance and reproducibility using both PP and PE meshes (FIG. 44).

### Example 71

### Preliminary Sterilization and Shelf Life Study

The effect of 2 sterilization methods, electron-beam (E-beam) and ethylene oxide (EtO), on the performance of adhesive-coated meshes was determined (Table 24). For formulations coated onto PE meshes, lap shear data revealed no statistical differences before and after E-beam sterilization (25 kGy). PP meshes were sterilized with EtO since irradiation has been found to cause chain scission of the polymer, reducing its strength. Although no statistical differences were observed before and after sterilization with EtO at 30° C., the oxidant embedded samples showed a decrease in lap shear adhesion strength. Additionally, these samples displayed a dark brown color, indicating pre-oxidation of the adhesive. This pre-oxidation is believed to be due to the high humidity associated with EtO sterilization.

TABLE 24

| Adhesive | | | | Sterilization | | Lap Shear | |
|---|---|---|---|---|---|---|---|
| Formulation | | Mesh Type | | Method | | Strength | |
| Average (kPa) | | | St. Dev. (kPa) | | Sample Size | | |
| Medhesive- | PE | | Non-sterile | 88.0 | 32.3 | | 30 |
| 137/138 | | | | | | | |
| Oxidant | | | | | | | |
| Embedded | | | | | | | |
| E-beam | | 128 | | 18.2 | | 6 | |
| Medhesive- | PE | | Non-sterile | 39.7 | 13.9 | | 28 |
| 132 | | | | | | | |
| E-beam | | 44.8 | | 9.43 | | 4 | |
| Medhesive- | PP | | Non-sterile | 56.0 | 11.6 | | 30 |
| 137/138 | | | | | | | |
| Oxidant | | | | | | | |
| Embedded | | | | | | | |
| EtO | | 30.4 | | 20.8 | | 6 | |
| Medhesive- | PP | | Non-sterile | 39.0 | 14.2 | | 28 |
| 132 | | | | | | | |
| EtO | | 38.4 | | 16.0 | | 6 | |

A preliminary shelf-life study was performed on E-beam sterilized samples. There were no statistical differences in terms of lap shear results for storage up to 22 and 35 days for E-beam-sterilized Medhesive-132 and oxidant embedded samples, respectively (Table 25). However, Medhesive-132 tested on day 22 showed an increase in variability of the lap shear data, while the oxidant embedded samples showed a drop in measured lap shear strength. These observations suggest that samples are negatively affected over time. Adhesives may be packaged in an air-permeable pouch, which exposes the adhesive to moisture and oxygen, both of which can lead to premature oxidation of the adhesive.

TABLE 25

| Adhesive | | Days Post | | | | |
|---|---|---|---|---|---|---|
| Formulation | | Sterilization | | Lap Shear Strength | | |
| Average (kPa) | | St. Dev. (kPa) | | Sample Size | | |
| Medhesive- | Non-sterile | 88.0 | | 32.3 | | 30 |
| 137/138 | | | | | | |
| Oxidant | | | | | | |
| Embedded | | | | | | |
| 8 | 69.7 | | 32.2 | | 8 | |
| 35 | 41.9 | | 12.2 | | 2 | |
| Medhesive- | Non-sterile | 39.7 | | 13.9 | | 30 |
| 132 | | | | | | |
| 2 | 44.8 | | 9.43 | | 4 | |
| 22 | 69.8 | | 43.0 | | 4 | |

### Example 72

### Bilateral Placement of Adhesive-Coated Meshes on the Dorsal Surface of the Intact Peritoneum of the Rabbit

Coated meshes remain fixed to the peritoneum over a 7-day period as assessed by possible construct detachment, migration, curled edges, and shrinkage. The biocompatibility of coated meshes with the surrounding tissues was monitored by adhesion formation, inflammatory response, and incorporation of the mesh into the abdominal wall.

Materials and Methods

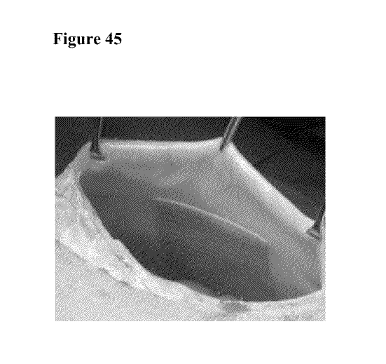

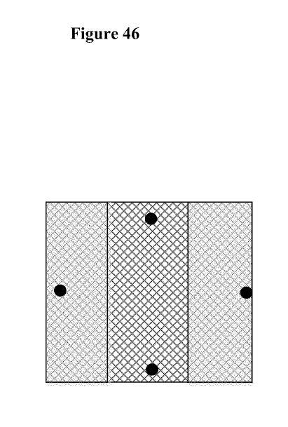

Six New Zealand white rabbits are used. A 10-cm midline incision is made in the abdominal wall to expose the peritoneum. Two 4×4 cm segments of adhesive-coated mesh are secured to the dorsal surface of the intact peritoneum (in an “underlay” position—(FIG. 45) one on each side of the incision. Two E-beam sterilized formulations, Medhesive-132 and Medhesive-137/138 (Table 26), are chosen. These adhesives are coated onto segments of light-weight polyester mesh according to the pattern in (FIG. 46) such that both ends of the segment of mesh are coated with adhesive, and the middle portion remains uncoated and accessible to tissue ingrowth. The oxidant for the adhesives are sodium periodate. For Medhesive-132, the oxidant is brushed onto the visceral side of the implanted porous mesh, and passes through the mesh onto the adhesive layer on the peritoneal side, thereby activating the adhesive. For the Medhesive-137/138 coating, the embedded oxidant is released through hydration of the films.

TABLE 26

| Animal | Animal's right side | Animal's left side |
|---|---|---|
| 1 | Mesh only + suture (no | M137/138 + suture (no |
| | adhesive) | oxidant) |
| 2 | M132 + oxidant + suture | M132 + oxidant |
| 3 | M132 + oxidant + suture | M132 + oxidant |
| 4 | M137/138 + oxidant + | M137/138 + oxidant |
| | suture | |
| 5 | M137/138 + oxidant + | M137/138 + oxidant |
| | suture | |
| 6 | M132 + oxidant | M137/138 + oxidant |

The fixation of the coated meshes in Table 26 is by adhesive alone, while the adhesive fixation of other coated meshes is reinforced on the four sides with non-absorbable sutures (black dots in FIG. 46). If mesh fixation is not maintained over the entire study period with adhesive alone, this additional fixation allows us to obtain histologic data regarding adhesion formation, tissue ingrowth and inflammation. The meshes fixed with adhesive alone are marked by non-absorbable sutures placed adjacent to the medial corners of the mesh. Using these suture markers enables determination if mesh migration has occurred. After mesh implantation, the wound is closed, and the animals are allowed to recover and are euthanized 7 days after surgery.

Assessments:

Assessments includes adhesion formation (which organs are involved, adhesion tenacity, % of mesh covered with adhesions), attachment of the constructs to the peritoneal wall, mesh migration, curling of mesh corners or edges, mesh shrinkage, degree of scar formation around and over the mesh, and histologic assessment of acute and chronic inflammation and tissue ingrowth into the mesh.

A confirmatory study in mini-pig is a clinically relevant animal model as hernia is created in these animals and repaired using our materials. synthetic mesh types (lightweight PP and PE) are used with satisfactory results. The optimal polymer formulation is applied to 2 representative synthetic hernia meshes rather than the 3 biologic meshes. There are 4 treatments (adhesive-coated mesh and mesh alone for the 2 mesh types) at the 2 time points (30 and 90 days), with 10 hernia sites per treatment/time point, or 80 total hernia sites.

The Robust Design technique is used to screen adhesive formulations based on lap shear adhesive performance, swelling, and degradation time. The effect of different factors such as film thickness, adhesive composition, oxidant type, and oxidant delivery method on these parameters is determined. Three formulations with optimal lap shear strength, a suitable degradation rate (1-3 months), and favorable cytotoxicity results are chosen for the further screening in a second preliminary animal study. The animal studies are used to screen adhesives for biocompatibility.

### Example 73

### Bioadhesive-Coated Scaffold Suitable for Achilles Tendon Repair

The Achilles tendon is the most frequently ruptured tendon, with an estimated 225,000 ruptures and 50,000 repairs of ruptures occurring annually in the US. Tendon ruptures, both acute and chronic (neglected), can dramatically affect a patient's quality of life, and require a prolonged period of recovery before return to pre-injury activity levels. While numerous surgical techniques and rehabilitative regimens have been proposed to shorten the recovery period without introducing additional complications, the standard of care remains primary suture repair. An adhesive-coated biologic membrane may be used to augment primary suture repair. The adhesive portion is a synthetic mimic of a mussel adhesive protein that can adhere to various surfaces in a wet environment, including biologic tissues. When combined with biologic membranes such as bovine pericardium or porcine dermal tissue for tendon repair, the adhesive constructs demonstrated adhesive strengths significantly higher than that of fibrin glue. Tensile mechanical testing of transected and repaired porcine tendons showed that suture repair augmented with these adhesive constructs exhibited increased stiffness (25-40%), failure load (24-44%), and energy to failure (27-63%) when compared to controls with suture repair alone. With further development, a pre-coated bioadhesive membrane may represent a potential new treatment option for Achilles tendon repair.

Achilles Tendon Repair

The Achilles tendon is ruptured more frequently than any other tendon. It accounts for 40-60% of all operative tendon repairs, with 75% of these procedures stemming from sports-related activities. (Leppilahti, 1998, Strauss, 2007, White, 2007) The number of ruptures has increased over the last several decades, and the rate has doubled nearly every 10 years. (Maffulli, 1999, Houshian, 1998, Pajala, 2002) The aging population, the increased popularity of recreational sports among the middle-aged, and medical advances that enable an aging population to participate in recreational sports all contribute to this increase. An estimated 50,000 surgical repairs of Achilles tendon ruptures are performed annually in the US, costing over $40,000 per case, including months of postoperative rehabilitation.

Primary suture repair is the current standard of care and many different suture techniques are available (e.g., Krackow, Bunnell, Kessler, 3-loop pulley, epitendinous suture augmentation). (Lee SJ, 2008, Lee SJ, 2009, Shepard, 2008, Pasternak, 2007, Korenkov, 2002, Herbort, 2008) However, primary repair of ruptured Achilles tendons has resulted in partial or complete re-ruptures in over 5% of patients. (Nistor, 1981, Winter, 1998) The suture-tendon junction is usually the weak link in primary tendon repairs due to the structure of tendinous tissue—the strength between the fibers is much less than that of the fibers themselves, so sutures can tear through the tendon when force is applied. (Kummer, 2005)

To reduce the rate of re-rupture and accelerate rehabilitation, primary suture repair is sometimes reinforced with biologic scaffolds or grafts (e.g., bovine pericardium, small intestinal submucosa (SIS), acellular human and porcine dermal matrix). (Gilber5, 2007, Liden, 2009) In addition to improved mechanical support, these biologic materials provide an extracellular matrix for the in-growth of tissue so that they become well-incorporated into the tendon. Patients augmented with biologic grafts were able to undergo an aggressive rehabilitation program and enjoyed early return-to-activity without rerupture or complications. (Lee DK, 2007, Lee DK, 2008) However, these grafts are secured to the tendon with suture which can cause local impairment of circulation with compromised healing. (Hohendorff, 2008, Hohendorff, 2009) Regardless of the treatment method, complete regeneration of the tendon is never achieved. (Tozer, 2005)

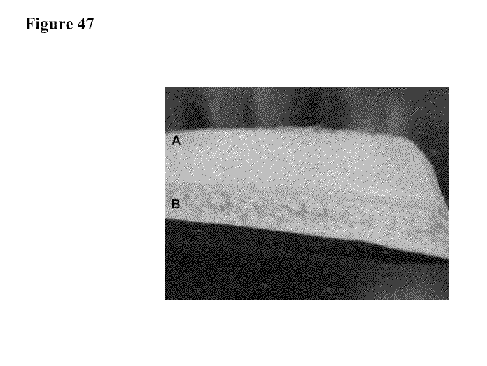

A further surgical option that can be used to augment primary suture repair of Achilles tendon ruptures is by affixing an adhesive-coated scaffold to the tendon surface. As shown in FIG. 47 a biological scaffold is pre-coated with a water-resistant adhesive that is a synthetic mimic of mussel adhesive proteins (MAPs) that allow marine mussels to bind tenaciously to various substrates in a wet, turbulent, and saline environment. (Waite, 1987, Yamamoto, 1996) A structural feature of MAPs is 3,4-dihydroxyphenylalanine (DOPA), an amino acid arising from post-translational modification of tyrosine. (Kramer, 1991) DOPA is a surface adhesion promoter and a crosslinking precursor. (Deming, 1999, Waite, 1991, Yu, 1999) Oxidation transforms DOPA into a reactive quinone that crosslinks with various functional groups (e.g., —NH₂, —SH) present on soft tissue surfaces. (Guvendiren, 2008, Lee H, 2006, Lee H 2007) Synthetic adhesives containing DOPA and its derivatives exhibit water-resistant adhesion to many surfaces (e.g., metal, soft tissues). (Brubaker, 2010, Burke, 2007, Lee BP, 2002, Lee BP, 2006)

Herein a MAP-mimetic synthetic adhesive was combined with either bovine pericardium or a commercial porcine dermal tissue (Biotape™, Wright Medical Technology, Inc), and characterized the adhesive properties of these adhesive constructs (AC). Additionally, tensile failure testing was performed on transected porcine tendons that had received primary suture repair with and without augmentation with these adhesive constructs.

Materials and Methods

Materials

The adhesive polymer, Medhesive-096, was prepared as described. Bovine pericardium was obtained from Nirod Corporation (Ames, Iowa), while Biotape™ was purchased from Wright Medical Technology, Inc. (Arlington, Tenn.). Porcine tendon (rear leg deep flexor) was purchased from Spear Products (Coopersburg, Pa.).

### Coating and Testing Adhesive-Coated Biologic Scaffolds

Solutions of Medhesive-096 (100 mg/mL in chloroform) were casted over bovine pericardium and Biotape, and then dried under vacuum overnight to create the adhesive-coated constructs denoted as AC1 and AC2, respectively. Lap shear testing was performed according to American Society for Testing and Materials (ASTM) standards (ASTM F2255). Wetted bovine pericardium was used as the tissue substrate. Prior to forming the adhesive joint, the adhesive was activated with a solution of NaIO₄ (20 mg/mL, 40 μL), compressed with a 100 g weight for 10 minutes, and further conditioned in phosphate buffered saline (PBS, pH 7.4, 37° C.) for an hour before testing. The adhesive joints were installed in the grips of a materials test machine (Admet, Inc., Norwood, Mass.), and loaded to failure at a rate of 10 mm/min. The maximum lap-shear strength needed to separate the adhesive joints was recorded. Commercially available tissue adhesives, Dermabond® (Ethicon Inc.) and Tisseel™ (Baxter Healthcare Corporation), were investigated for comparison purposes. The adhesives were applied in situ according to the manufacturer's instructions. The sample size was 6 in each test group.

Mechanical Testing of Repaired Tendons

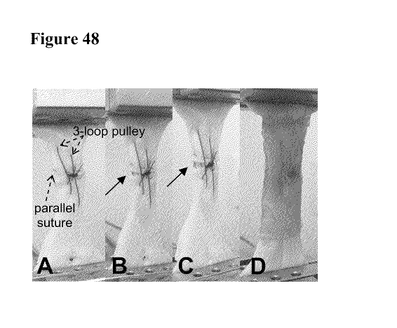

Tensile failure testing was performed on transected tendons repaired using a suture technique with and without augmentation with the proposed adhesive-coated meshes. Transected porcine tendons were sutured with both parallel (Polysorb™ Braided Lactomer™ 4-0, Covidien) and 3-loop pulley (Maxon™ monofilament polyglyconate, 0, Covidien) suture patterns (FIG. 48). The parallel sutures (horizontal) were used to keep the two ends of the transected tendon in intimate contact in order to minimize gap formation, while the 3-loop pulley (vertical) was intended to be the main structural component that held the severed tendon together. The adhesive construct was first secured to the tendon with three stay sutures, and then a solution of NaIO₄ (20 mg/mL) was sprayed onto the adhesive prior to wrapping it around the tendon, to activate the adhesive. AC1 was wrapped around the tendon twice whereas AC2 was wrapped around once with 1-cm of overlap. The wrapped tendons were held tightly for 10 min and incubated at 37° C. (PBS, pH 7.4) for 1 hour prior to testing. After preconditioning the repaired tendons (cycled 10 times between 2 to 10 N), both sutured tendons and adhesive-wrapped tendons were loaded to failure at a rate of 25 mm/min, and load vs. displacement data were recorded. The initial grip length of 6 cm was used to compute strain. The failure load was determined to be the load where the parallel sutures began to fail—where irreversible failure of the repair occurred. The stiffness of the repair was determined from the slope of the linear portion of the load vs. strain curve, and the energy to failure was determined from the area under the load vs. strain curve up to the failure load. The sample size was 10 for each test group.

Statistical Analysis

Mechanical data resulting from treatments in lap shear testing and tendon repair testing were compared using analysis of variance (ANOVA) and Tukey post hoc analysis with a significance level of p=0.05.

Results

Adhesive Properties of Novel Adhesive Constructs

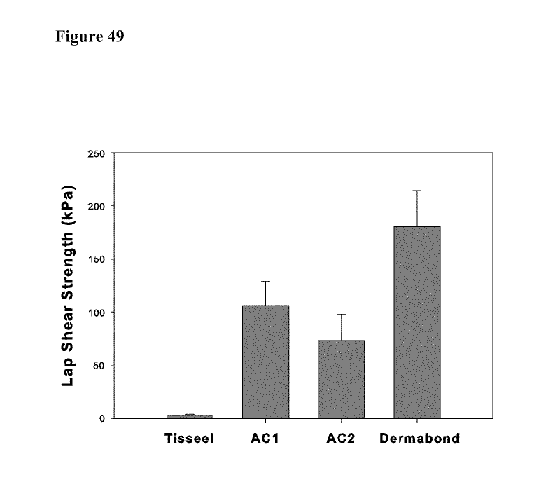

Lap shear adhesion testing (FIG. 49) demonstrated that both adhesive constructs exhibited failure strengths that were 28-40 times greater than that of fibrin glue (Tisseel). While Dermabond exhibited the highest adhesive strength among the adhesives tested, cyanoacrylate-based adhesives have safety concerns (Sierra, 1996, Ikada, 1997, Bilic, 2010) and can dramatically alter the biomechanical properties of the repaired tissues. (Fortelny, 2007) Both AC1 and AC2 were used in subsequent testing to augment the suture repair of transected tendons.

Mechanical Testing of Repaired Tendons

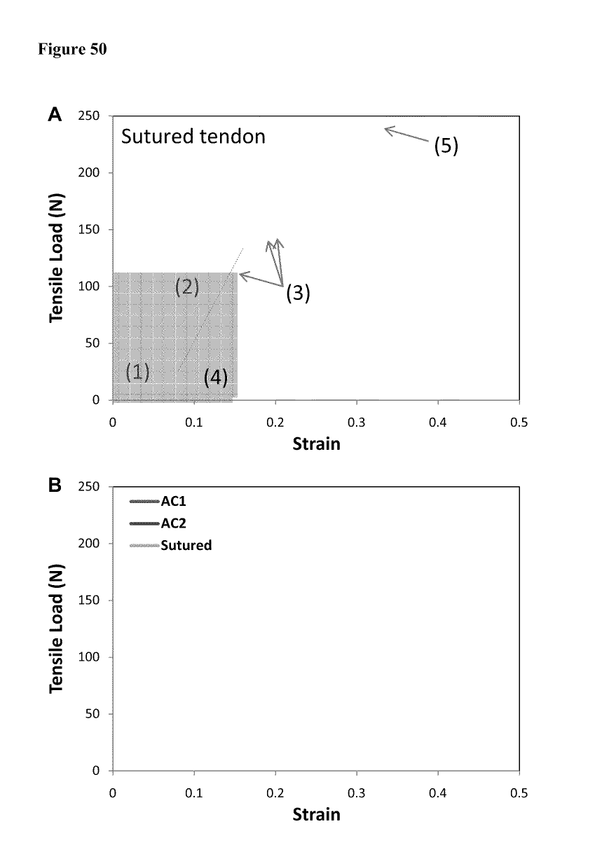

FIG. 50A shows a representative load vs. strain curve for a sutured tendon, which contains typical features that were evident in all test groups (FIG. 50B) (1) non-linear toe region where the fibers are being recruited as the tendon is stretched, (2) linear region representing the linear stiffness of the repaired tendon, (3) arrows pointing to reduction in the load corresponding with the parallel sutures being pulled off the tendon, with the first of these instances being considered as the irreversible failure of the repair (failure load), (4) the area under the load-strain curve up to the failure load, used to calculate energy to failure, and (5) peak load where the 3-loop pulley began to fail as it is pulled through the tendon.

Both AC1- and AC2-augmented tendons exhibited greater load to failure (24-44%), stiffness (25-39%), and energy to failure (27-63%), compared with suture-only controls (Table 27).

TABLE 27

| | | | |
|---|---|---|---|
| Linear Stiffness (N) | 1045 ± 305 | 1451 ± 254* | 1305 ± 340^{#} |
| Load to Failure (N) | 105 ± 25.1 | 151 ± 37.4* | 130 ± 45.5^{#} |
| Strain to Failure | 0.158 ± 0.0208 | 0.159 ± 0.0318 | 0.159 ± 0.0298 |
| Energy to Failure (J) | 0.386 ± 0.131 | 0.630 ± 0.194* | 0.492 ± 0.236 |
| Peak Load (N) | 217 ± 45.7 | 231 ± 35.6 | 245 ± 35.8 |
| Strain @ Peak Load | 0.356 ± 0.0602 | 0.370 ± 0.0612 | 0.380 ± 0.0606 |

*p < 0.05 compared to suture only;
^{#}p < 0.15 compared to suture only.
n = 10 replicates per treatment.

These differences were statistically significant for AC1 (p<0.05). While suture-only tendons readily formed a gap at the transected site at loads as low as 10 N no visible gap was formed in AC1-wrapped tendons until failure. Gap formation has been attributed to inflammation and inadequate healing as a result of poorly aligned collagen fibers. The strains to failure for all test groups were not statistically different, indicating that the parallel sutures begin to fail when tendons were being loaded to the same strain, regardless of treatment. Similarly, peak loads were not statistically different between the three test groups. While the 3-loop suture is the primary structural component that holds the tendon together, irreversible failure had already occurred when the parallel sutures were pulled out of the tendons. Initial failure load, and not peak load, is likely the more important failure metric when considering repeated loading of a healing tendon.

Bio-Adhesive Tendon Repair

A synthetic bioadhesive is utilized herein as an adhesive coating for securing surgical graft material to Achilles tendons. This coating contains an active adhesive functional group, dopamine, which resembles the catecholic side chain of DOPA that marine mussels utilize to form strong bonds in the presence of water. Other dopamine-modified synthetic polymers have strong adhesive properties. Catechol's ability to crosslink is exploited with both the biologic mesh and tissue substrate to generate interfacial bonds. Catechols are oxidized to form highly reactive quinones, which form covalent crosslinking with other catechols within the adhesive film (cohesive crosslinking) or functional groups such as amine and thiol found on tissue surfaces (adhesive crosslinking).

The adhesive catechol is chemically attached to biocompatible and biodegradable multiblock copolymers consisting of poly(ethylene glycol) (PEG) and polycaprolactone (PCL). The presence of PEG allows the adhesive polymer to remain relatively hydrophilic in order to achieve good “wetting” or adhesive contact with a biologic mesh or tissue substrate, while the hydrophobic PCL segments increase cohesive strength and prevent rapid dissolution of the film in the presence of water. The adhesive film degrades through hydrolysis of ester linkages in PCL (20% mass loss over 5 months in vitro).

The adhesive polymer was solvent casted onto two biologic scaffolds to demonstrate the feasibility of using the adhesive-coated construct in Achilles tendon repair. Bovine pericardium was chosen as one of the backing materials because it is an inexpensive and readily abundant extracellular matrix with suitable mechanical properties (tensile failure load of 41±9.8 N/cm).

Clinical Uses

The adhesive-coated biologic membrane is a treatment option for surgical repair of Achilles tendon ruptures. AC-reinforced tendons exhibited significantly higher stiffness, load to failure, and energy to failure, as well as reduced gap formation, when compared to primary suture repair alone. A more mechanically secure fixation method may allow patients with adhesive-wrapped tendon repairs to initiate a rehabilitation program at an earlier time point or perform a more aggressive rehabilitation regimen. Conventional postoperative treatment for surgically repaired Achilles tendons has meant immobilization in a below-the-knee plaster cast for six to eight weeks with little to no weight-bearing. However, complications of prolonged immobilization include muscle atrophy, joint stiffness, tendocutaneous adhesions, deep vein thrombosis, and ulceration of joint cartilage. Recent clinical studies, including randomized controlled follow-ups, strongly suggest that early mobilization and weight-bearing, when compared with immobilization, produce less tendon elongation, greater isokinetic calf muscle strength, improved quality of life, and more rapid resumption of normal activities without rerupture.

Various biologic scaffolds or meshes (e.g., bovine pericardium, small intestinal submucosa, acellular human and porcine dermal matrix) have been evaluated for tendon repair augmentation. In addition to mechanical support, these biologic graft materials serve as a matrix for tissue in-growth, and have become well-incorporated into the tendon tissue in animal models and clinically. An augmented repair allowed patients to undergo an aggressive rehabilitation program with subsequent early return-to-activity without rerupture or complications. However, these non-adhesive biologic grafts require multiple intratendinous, interlocking sutures placed throughout the construct to prevent motion along the tendon/graft interface, thereby potentially disrupting local blood flow. The adhesive-coated constructs reported herein reduce the number of sutures or completely replace the use of sutures in graft fixation.

The adhesive performance of a biologically-inspired synthetic adhesive coated onto two biologic membranes, bovine pericardium and Biotape, was compared. These adhesive-coated constructs demonstrated significantly higher adhesive strength compared with commercial fibrin glue. Tensile mechanical testing was performed on transected porcine Achilles tendons with primary suture repair with or without adhesive construct reinforcement. Tendons augmented with AC wraps exhibited elevated stiffness, failure load, and energy to failure, as well as reduced gap formation, compared with the suture-only controls.

### REFERENCES

Leppilahti J, Orava, S. Total Achilles Tendon Rupture: A Review. Sports Med. 1998; 25(2):79-100.
Strauss E J, Ishak, C., Jazrawi, L., Sherman, O., Rosen, J. Operative Treatment of acute Achilles tendon ruptures: An institutional review of clinical outcomes. Injury, International Journal of the care of the Injured. 2007; 38:832-8.
White D W, Wenke J C, Mosely D S, Mountcastle S B, Basamania C J. Incidence of major tendon ruptures and anterior cruciate ligament tears in US army soldiers. Am J Sports Med. 2007; 35(8):1308-14.
Maffulli N. Rupture of the Achilles Tendon. The Journal of Bone and Joint Surgery. 1999; 81-A(7):1019-36.
Houshian S, Tscherning T, Riegels-Nielsen P. The epidemiology of Achilles tendon rupture in a Danish county. Injury. 1998; 29:651-4.
Pajala A, Kangas, J., Ohtonen, P., Leppilahti, J. Rerupture and Deep Infection Following Treatment of Total Achilles Tendon Rupture. The Journal of Bone and Joint Surgery. 2002; 84-A(11):2016-21.
Williams G R J, et al. Rotator Cuff Tears: Why do we repair them? The Journal of Bone and Joint Surgery. 2004; 86-A(12):2764-76.
Lee S J, Goldsmith S, Nicholas S J, McHugh M, Kremenic I, Ben-Avi S. Optimizing Achilles tendon repair: effect of epitendinous suture augmentation on the strength of achilles tendon repairs. Foot Ankle Int 2008; 29(4):427-32.
Lee S J, Sileo M J, Kremenic I J, Orishimo K, Ben-Avi S, Nicholas S J, et al. Cyclic loading of 3 Achilles tendon repairs simulating early postoperative forces. Am J Sports Med. 2009; 37(4):786-90.
Shepard M E, Lindsey D P, Chou L B. Biomechanical comparison of the simple running and cross-stitch epitenon sutures in achilles tendon repairs. Foot Ankle Int. 2008; 29(5):513-7.
Pasternak B, Missios A, Askendal A, Tengvall P, Aspenberg P. Doxycycline-coated sutures improve the suture-holding capacity of the rat Achilles tendon. Acta Orthop Scand. 2007; 78(5):680-6.
Korenkov M, Sauerland, S., Arndt, M., Bograd, L., Neugebauer, E. A. M., Troidl, H. Randomized clinical trial of suture repair, polypropylene mesh or autodermal hernioplasty for incisional hernia. British Journal of Surgery. 2002; 89:50-6.
Herbort M, Haber A, Zantop T, Gosheger G, Rosslenbroich S, Raschke M J, et al. Biomechanical comparison of the primary stability of suturing Achilles tendon rupture: a cadaver study of Bunnell and Kessler techniques under cyclic loading conditions. Arch Orthop Trauma Surg. 2008; 128(11):1273-7.
Nistor L. Surgical and Non-surgical Treatment of Achilles Tendon Rupture. The Journal of Bone and Joint Surgery. 1981; 63-A(3):394-9.
Winter E, Weise, K., Weller, S., Ambacher, T. Surgical Repair of Achilles Tendon Rupture: Comparison of Surgical with Conservative Treatment. Arch Orthop Trauma Surg. 1998; 117:364-7.
Kummer F J, Iesaka, K. The Role of Graft Materials in Suture Augmentation for Tendon Repairs and Reattachment. J Biomed Mater Res Part B: Appl Biomater. 2005; 74B:789-91.
Gilbert T W, Stewart-Akers A M, Simmons-Byrd A, Badylak S F. Degradation and Remodeling of Small Intestinal Submucosa in Canine Achilles Tendon Repair. The Journal of Bone and Joint Surgery (American). 2007; 89:621-30.
Liden B A, Simmons M. Histologic evaluation of a 6-month GraftJacket matrix biopsy used for Achilles tendon augmentation. J Am Podiatr Med. Assoc. 2009; 99(2):104-7.
Lee D K. A preliminary study on the effects of acellular tissue graft augmentation in acute Achilles tendon ruptures. J Foot Ankle Surg. 2008 47(1):8-12.
Hohendorff B, Siepen W, Spiering L, Staub L, Schmuck T, Boss A. Long-term results after operatively treated Achilles tendon rupture: Fibrin glue versus suture. J Foot Ankle Surg. 2008; 47(5):392-99.
Hohendorff B, Siepen W, Staub L. Treatment of acute Achilles tendon rupture: Fibrin glue versus fibrin glue augmented with plantaris longus tendon. J Foot Ankle Surg. 2009; 48(4):439-46.
Tozer S, Duprez, D. Tendon and Ligament: Development, Repair and Disease. Birth Defects Research (Part C). 2005; 75:226-36.
Waite J H. Nature's underwater adhesive specialist. Int J Adhes Adhes. 1987; 7(1):9-14.
Yamamoto H. Marine adhesive proteins and some biotechnological applications. Biotechnol Genet Eng Rev. 1996; 13:133-65.
Kramer K J, Morgan T D, Hopkins T L, Christensen A, Schaefer J. Insect cuticle tanning: Enzymes and cross-link structure. Natural Occurring Pest Bioregulators; 1991. p. 87-105.
Deming T J, Yu M, Hwang J. Mechanical studies of adhesion and crosslinking in marine adhesive protein analogs. Polymeric Materials: Science and Engineering. 1999; 80:471-2.
Waite J H. Mussel beards: A coming of Age. Chemistry and Industry. 1991 2 Sept.; 2 Sept.:607-11.
Yu M, Hwang J, Deming T J. Role of L-3,4-dihydroxyphenylanine in mussel adhesive proteins. Journal of American Chemical Society. 1999; 121(24):5825-6.
Guvendiren M, Messersmith P B, Shull K R. Self-Assembly and Adhesion of DOPA-Modified Methacrylic Triblock Hydrogels. Biomacromol.
Lee H, Scherer N F, Messersmith P B. Single Molecule Mechanics of Mussel Adhesion. Proc Natl Acad. Sci. 2006; 103:12999-3003.
Brubaker C E, Kissler H, Wang L-J, Kaufman D B, Messersmith P B. Biological performance of mussel-inspired adhesive in extrahepatic islet transplantation. Biomaterials. 2010; 31:420-7.
Burke S A, Ritter-Jones M, Lee B P, Messersmith P B. Thermal gelation and tissue adhesion of biomimetic hydrogels. Biomed Mater. 2007; 2:203-10.
Lee B P, Dalsin J L, Messersmith P B. Synthesis and Gelation of DOPA-Modified Poly(ethylene glycol) Hydrogels. Biomacromol. 2002; 3(5):1038-47.
Lee B P, Chao C-Y, Nunalee F N, Shull K R, Messersmith P B. Rapid Photocurable of Amphiphilic Block Copolymers Hydrogels with High DOPA Contents. Maclomolecules. 2006; 39:1740-48.
Sierra D, Saltz R. Surgical Adhesives and Sealants: Current Technology and Applications. Lancaster, Pa.: Technomic Publishing Company, Inc; 1996.
Ikada Y. Tissue adhesives. In: Chu C C, von Fraunhofer J A, Greisler H P, editors. Wound Closure Biomaterials and Devices. Boca Raton, Fla.: CRC Press, Inc.; 1997. p. 317-46.
Bilic G, Brubaker C, Messersmith P B, Mallik A S, Quinn T, Done E, et al. Injectible candidate sealants for fetal membrane repair: Bonding and toxicity in vitro. American Journal of Obstetrics and Gynecology. 2010; 202(85):1-9.
Fortelny R H, Petter-Puchner A H, Walder N, Mittermayr R, Öhlinger W, Heinze A, et al. Cyanoacrylate tissue sealant impairs tissue integration of macroporous mesh in experimental hernia repair Surgical Endoscopy. 2007; 21(10):1781-5.
Lee H, Lee B P, Messersmith P B. A Reversible Wet/Dry Adhesive Inspired by Mussels and Geckos. Nature. 2007; 448(19 July):338-41.

### Example 74

### Effect of Formulation on Degradation Rate

In this example, sample adhesives were incubated in 15 mL of 1×PBS buffer at 37 or 55° C., respectively (Table 28).

TABLE 28

| | | Degradation | | Days to |
|---|---|---|---|---|
| | | Temperature | | 100% |
| Compound(s) | NalO₄:HFA | (C.) | Sterilization | Degradation |
| Med-141/142 | 2.8:1 | 55 | No | 13 |
| Med-141/142 | 2.8:1 | 37 | No | 63 |
| Med-141/142 | 2.1:1 | 55 | No | 8-11 |
| Med-141/142 | 2.1:1 | 37 | No | 56-66 |
| Med-141/142 | 2.1:1 | 55 | Yes | 9-11 |
| Med-141/142 | 2.1:1 | 37 | Yes | 47-55 |
| Med-141/142 | 1.4:1 | 55 | No | 8 |
| Med-141/142 | 1.4:1 | 37 | No | 44-49 |
| Med-141/142 | 1.4:1 | 55 | Yes | 8-10 |
| Med-141/142 | 1.4:1 | 37 | Yes | 42-49 |

### Example 75

### Adhesive Strength

In this Example, 9 separate batches of Medhesive-141/142 were tested 30 times each for n=270 at an oxidant concentration of 2.8:1 NaIO₄:HFA. Peak Load was observed to be (N)=27.45+/−9.43 N(CV=34.37%). Peak Stress was observed to be (kPa)=106.52+/−36.84 N(CV=34.58%)

### Example 76

### Oxidant Concentration

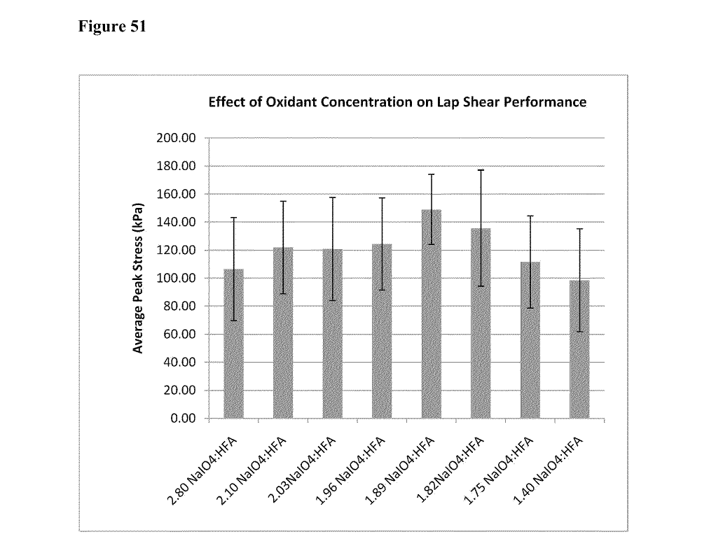

In this example the of varying oxidant concentration (for n≧12) was observed to demonstrate no statistical difference over the concentrations tested (FIG. 51).

### Example 77

### Effect of Oxidant Concentration on Swelling

In this example, the effect of oxidant concentration on the swelling ratio of Mehesive 141/142 were tested (Table 30)

TABLE 29

| Compound(s) | NalO₄:HFA | Swelling Ratio | Sterilization |
|---|---|---|---|
| Med-141/142 | 1.4:1 | 1.85 +/− 0.11 | No |
| Med-141/142 | 1.4:1 | 1.63 +/− 0.24 | Yes |
| Med-141/142 | 2.1:1 | 1.22 +/− 0.28 | No |
| Med-141/142 | 2.1:1 | 1.39 +/− 0.10 | Yes |
| Med-141/142 | 2.8:1 | 2.13 +/− 0.30 | No |

### Example 78

### In Vivo Testing in an Inguinal Porcine Model Methods

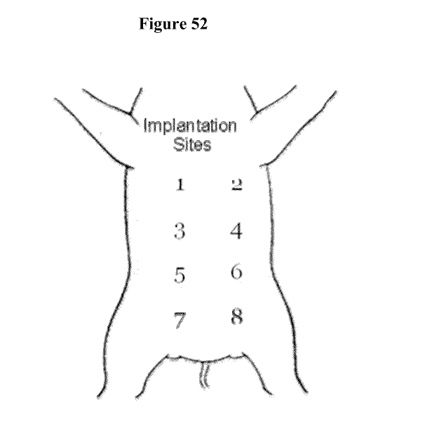

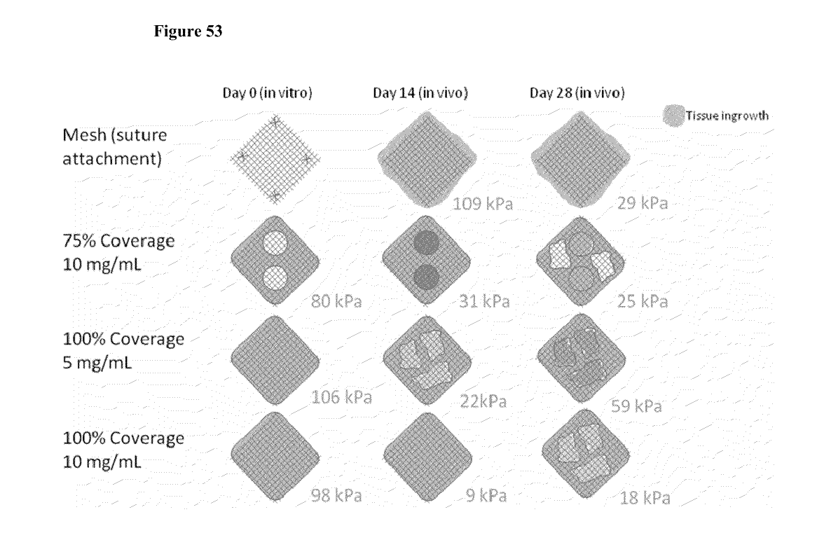

2″×3″ polyester meshes meshes were coated with adhesive in a pattern (75% coverage) and throughout the entirety of the mesh (100% coverage). Additionally, oxidant was varied from 2.8:1 NaIO₄:HFA (10 mg/mL) to 1.4:1 NaIO₄:HFA (5 mg/mL). Implantation sites are depicted in FIG. 52. The adhesive characteristics of the material were tested by pulling on the 2″×3″ polyester mesh using a hand held tensile tester. The peak load registered on the tensile tester was then normalized by the surface area of the mesh attached between the peritoneum and muscle/fascia layer. The testing was performed at necropsy at Days 14 and 28 and these results were compared to testing in vitro at Day 0 (FIG. 53).

Results

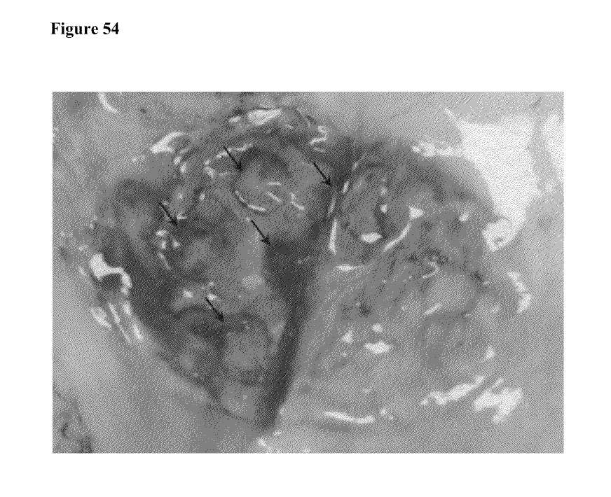

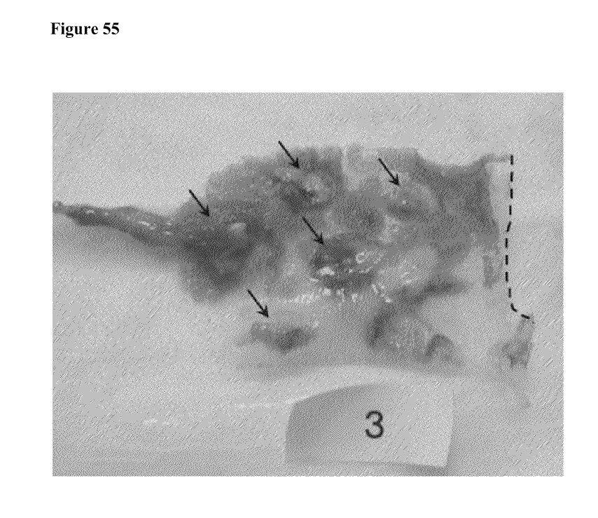

At day 14, one pig was euthanized and the implant site was explanted (FIG. 54). An edge of the adhesive construct was separated from the tissue and the construct was pulled with a handheld tensile tester until failure. The tensile load needed to separate the patterned adhesive coated mesh from the tissue was measured to be 54.6 N, which resulted in mesh failure. The portion of the mesh that remain attached to the tissue was subjected to a second tensile testing, requiring 66.7 N to be completely detached. There was significant amount of ingrowth in the regions not coated with adhesive where the tissue remained attached to the mesh (FIG. 55).

### Example 79

### Extraperitoneal Implantation of Adhesive Mesh with Embedded Oxidant

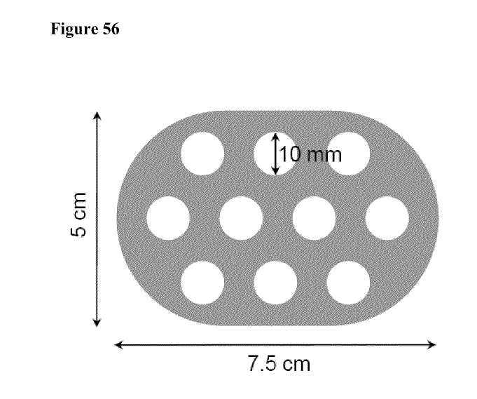

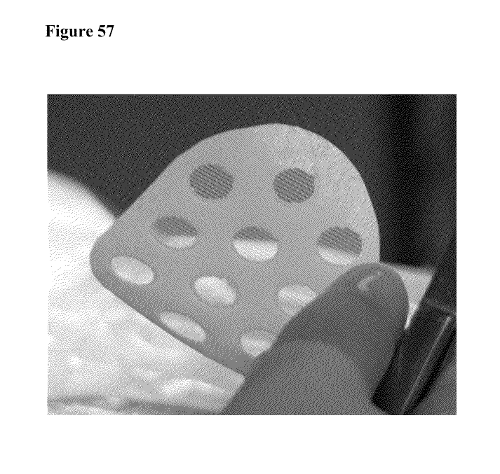

3 samples (Table 31) of 5×7.5 cm (oval-shaped) adhesive-coated meshes were implanted extraperitoneally in a porcine model (2 pigs). PE mesh was sandwiched between a layer of Medhesive-141 (240 g/m²) and Medhesive-142 (120 g/m²) embedded with oxidant (NaIO₄). One of the three samples had patterns of 5-mm circles not coated with Medhesive-141 and Medhesive-142 for rapid tissue ingrowth (FIG. 56, FIG. 57).

TABLE 30

| | | | NaIO₄ |
|---|---|---|---|
| | | | Concentration |
| Sample | Adhesive | Pattern | (g/m²) |
| Control | No adhesive, Sutured | No | No |
| 25015A | Yes | No | 14 |
| 25016A | Yes | No | 7.1 |
| 25014A | Yes | Yes (75% surface | 14 (75% coverage) |
| | | coverage w/ | |
| | | adhesive) | |

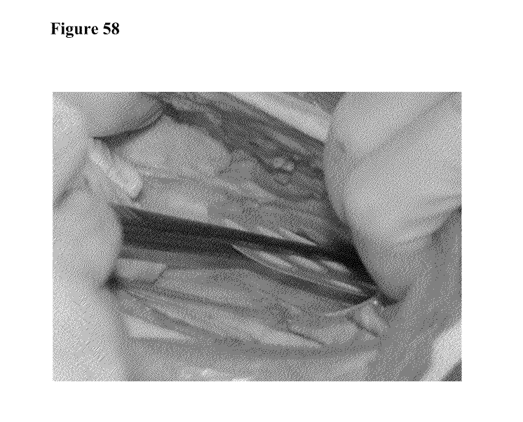

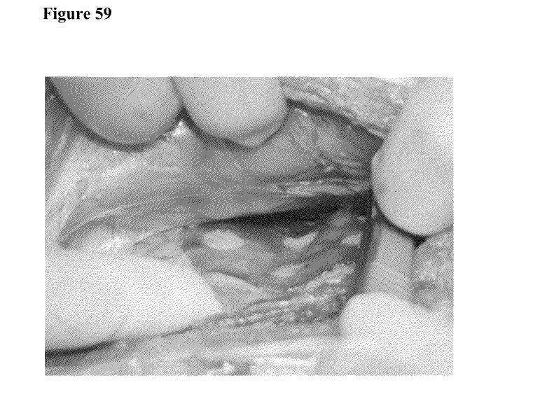

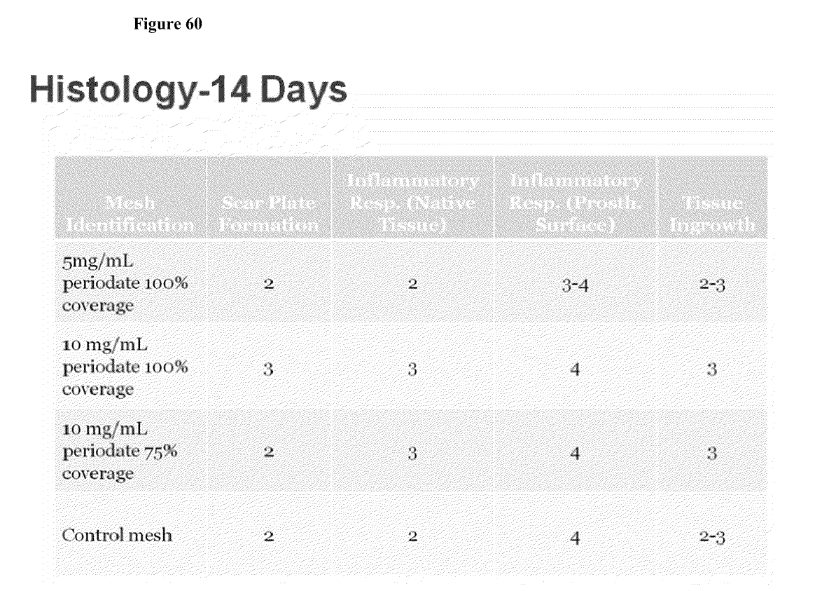

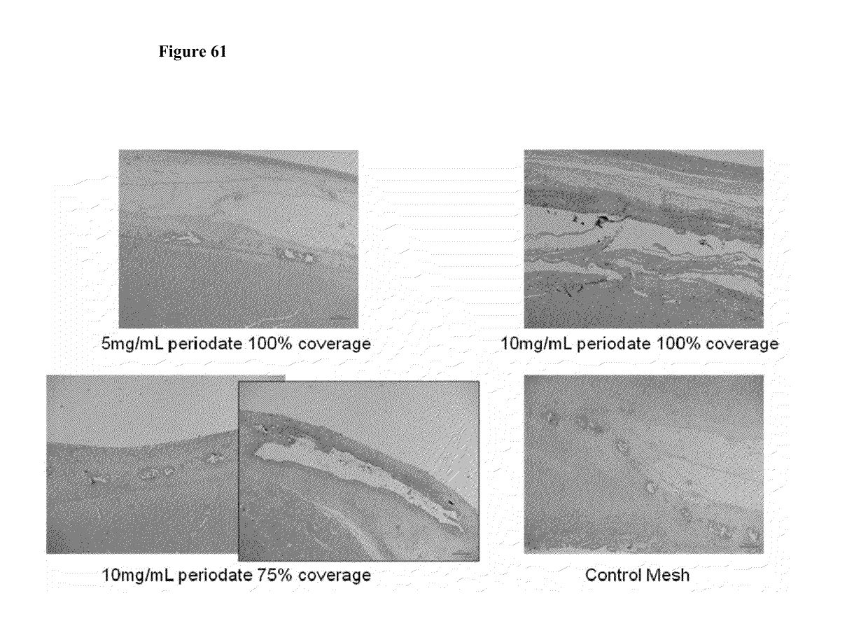

The samples were placed directly on the surgically exposed peritoneal surface of the animal, in bilateral rows of four each in a discrete tissue pocket between the peritoneum and muscle/fascial layer. The positioning of the medial side of the mesh was marked by placing a surgical staple in the overlying muscle tissue. The dry adhesive-coated meshes were placed in the tissue pocket and held with digital pressure for 5 minutes (FIG. 58). The adhesive was activated with the moisture in the tissue, which dissolved and released the oxidant during hydration (FIG. 59). Control PE meshes were sutured to peritoneum. The animals are euthanized at days 14 and 28, and the test articles are subjected to gross, mechanical, and histological evaluation of tissue response and initial tissue ingrowth. Day 14 histologic results are shown in FIG. 60, and photomicrographs of an the inguinal porcine model are shown in FIG. 61.

Although the present invention has been described with reference to preferred embodiments, persons skilled in the art will recognize that changes may be made in form and detail without departing from the spirit and scope of the invention. All references cited throughout the specification, including those in the background, are incorporated herein in their entirety. Those skilled in the art will recognize, or be able to ascertain, using no more than routine experimentation, many equivalents to specific embodiments of the invention described specifically herein. Such equivalents are intended to be encompassed in the scope of the following claims.

What is claimed is:

1. A compound comprising the formula (I) 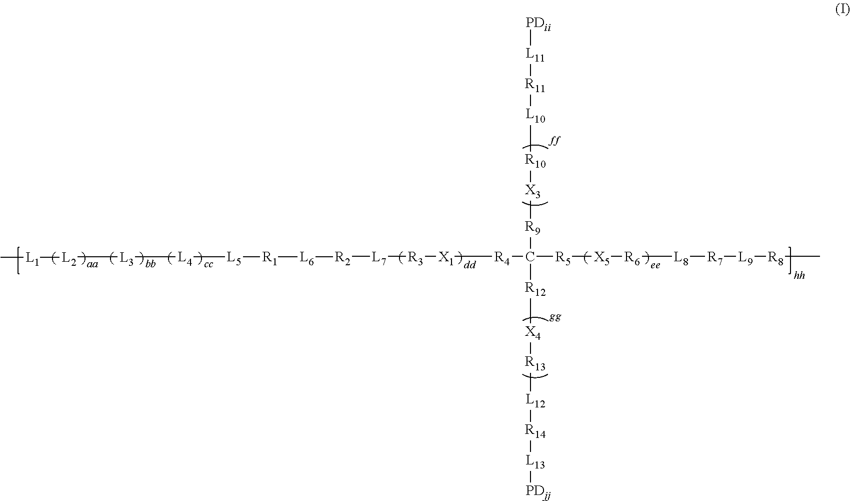
wherein
each L₂, L₃ and L₄ independently, is a linker;
each L₁, L₅, L₆, L₇, L₈, L₉, L₁₀, L₁₁ L₁₂ and L₁₃, independently, is a linker or a suitable linking group selected from amine, amide, ether, ester, urea carbonate or urethane linking groups;
each X₁, X₂, X₃ and X₄ independently, is an oxygen atom or NR;
R, if present, is H or a branched or unbranched C₁-C₁₀ alkyl group;
each R₁, R₂, R₃, R₄, R₅, R₆, R₇, R₈, R₉, R₁₀, R₁₁, R₁₂, R₁₃ and R₁₄ independently, is a branched or unbranched C1-C15 alkyl group;
each PDᵢᵢ and PDⱼⱼ, independently, is a phenyl derivative residue;
aa is a value from 0 to about 80;
bb is a value from 0 to about 80;
cc is a value from 0 to about 80;
dd is a value from 1 to about 120;
ee is a value from 1 to about 120;
ff is a value from 1 to about 120;
gg is a value from 1 to about 120; and
hh is a value from 1 to about 80.

2. The compound of claim 1, wherein L₂ is a residue of a C1-C15 alkyl lactone or lactam, a poly C1-C15 alkyl lactone or lactam, a polyester, or a compound comprising the formula Y₄—R₁₇—C(═O)—Y₆, wherein Y₄ is OH, NHR, a halide, or an activated derivative of OH or NHR; R₁₇ is a branched or unbranched C1-C15 alkyl group; and Y₆ is NHR, a halide, or OR.

3. The compound of claim 2, wherein the polylactone is a polycaprolactone.

4. The compound of claim 1, wherein L₃ is a residue of an alkylene diol, an alkylene diamine or a poly(alkylene oxide) polyether or derivative thereof.

5. The compound of claim 4, wherein L₃ is a poly(alkylene oxide) or —O—CH₂CH₂—O—CH₂CH₂—O—.

6. The compound of claim 1, wherein L₂ or L₄ is a residue of a C1-C15 alkyl lactone or lactam, a poly C1-C15 alkyl lactone or lactam, or a compound comprising the formula Y₄—R₁₇—C(═O)—Y₆, wherein Y₄ is OH, NHR, a halide, or an activated derivative of OH or NHR; R₁₇ is a branched or unbranched C1-C15 alkyl group; and Y₆ is NHR, a halide, or OR.

7. The compound of claim 6, wherein the polylactone is polycaprolactone.

8. The compound of claim 1, wherein X₁, X₂, X₃ and X₄ are each O or NH.

9. The compound of claim 1, wherein R₃, R₆, R₁₀ and R₁₃ are each —CH₂CH₂—.

10. The compound of claim 1, wherein X₁, X₂, X₃ and X₄ are each O.

11. The compound of claim 1, wherein R₄, R₅, R₉ and R₁₂ are each —CH₂—.

12. The compound of claim 1, wherein R₁, R₂, R₇, R₈, R₁₁ and R₁₄ are a branched or unbranched alkane.

13. The compound of claim 16, wherein R₁, R₂, R₇, R₈, R₁₁ and R₁₄ are —CH₂—CH₂— or CH₂—CH₂—CH₂—.

14. The compound of claim 1, wherein L₁, L₅, L₆, L₇, L₈, L₉, L₁₀, L₁₁, L₁₂, and L₁₃ form an amide, ester or carbamate.

15. The compound of claim 1, wherein each PDₓₓ and PD_{dd}, independently, is a residue of a formula comprising: 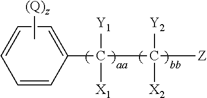
wherein Q is an OH or OCH3;
“z” is 1 to 5;
Each X₁, independently, is H, NH₂, OH, or COOH;
Each Y₁, independently, is H, NH₂, OH, or COOH;
Each X₂, independently, is H, NH₂, OH, or COOH;
Each Y₂, independently, is H, NH₂, OH, or COOH;
Z is COOH, NH₂, OH or SH;
aa is a value of 0 to about 4;
bb is a value of 0 to about 4; and
optionally provided that when one of the combinations of X₁ and X₂, Y₁ and Y₂, X₁ and Y₂ or
Y₁ and X₂ are absent, then a double bond is formed between the Cₐₐ and C_{bb}, further provided that aa and bb are each at least 1 to form the double bond when present.

16. The compound of claim 1, wherein PDₓₓ and PD_{dd} residues are selected from the group consisting of 3,4-dihydroxyphenylalanine (DOPA), 3,4-dihydroxyphenethylamine (dopamine), 3,4-dihydroxyhydrocinnamic acid (DOHA), 3,4-dihydroxyphenyl ethanol, 3,4-dihydroxyphenylacetic acid, 3,4-dihydroxyphenylamine, 3,4-dihydroxybenzoic acid, 3-(3,4-dimethoxyphenyl)propionic acid, 3,4-dimethoxyphenylalanine, 2-(3,4-dimethoxyphenyl)ethanol, 3,4-dimethoxyphenethylamine, 3,4-dimethoxybenzylamine, 3,4-dimethoxybenzyl alcohol, 3,4-dimethoxyphenylacetic acid, 3-(3,4-dimethoxyphenyl)-2-hydroxypropanoic acid, 3,4-dimethoxybenzoic acid, 3,4-dimethoxyaniline, 3,4-dimethoxyphenol, 3-(4-Hydroxy-3-methoxyphenyl)propionic acid, homovanillyl alcohol, 3-methoxytyramine, 3-methoxy-L-tyrosine, homovanillic acid, 4-hydroxy-3-methoxybenzylamine, vanillyl alcohol, vanillic acid, 5-amino-2-methoxyphenol, 2-methoxyhydroquinone, 3-hydroxy-4-methoxyphenethylamine, 3-hydroxy-4-methoxyphenylacetic acid, 3-hydroxy-4-methoxyphenylacetic acid, 3-hydroxy-4-methoxybenzylamine, 3-hydroxy-4-methoxybenzyl alcohol, isovanillic acid.

17. The compound of claim 1, wherein
L₂ is a residue of a polycaprolactone, a caprolactone, a polylactic acid, a polylactone or a lactic acid or lactone;
L₃ is a residue of polyethylene glycol;
L₄ is a residue of a polycaprolactone, a caprolactone, a polylactic acid, a polylactone or a lactic acid or lactone;
X₁, X₂, X₃ and X₄ are each O or NH;
R₁, R₃, R₆, R₈, R₁₀, and R₁₃ are each —CH₂CH₂—;
R₄, R₅, R₉ and R₁₂ are each —CH₂—;
R₂, R₇, R₁₁ and R₁₄ are each —(CH₂)ₙ—, wherein n is 3;
L₁, L₅, L₇, L₈, L₁₀, L₁₂ form an ester;
L₆, L₉, L₁₁, and L₁₃ form an amide; and
PDₓₓ and PD_{dd} are residues selected from the group consisting of 3,4-dihydroxyhydrocinnamic acid (DOHA), hydroferulic acid (HFA), or 3,4-dimethoxyhydrocinnamic acid (3,4-DMHCA).

18. The compound of claim 1, wherein
L₂ is a residue of a polycaprolactone, a caprolactone, a polylactic acid, a polylactone or a lactic acid or lactone;
L₃ is a residue of polyethylene glycol;
L₄ is a residue of a polycaprolactone, a caprolactone, a polylactic acid, a polylactone or a lactic acid or lactone;
X₁, X₂, X₃ and X₄ are each O or NH;
R₃, R₆, R₁₀, and R₁₃ are each —CH₂CH₂—;
R₁, R₈, R₄, R₅, R₉ and R₁₂ are each —CH₂—;
R₂, R₇, R₁₁ and R₁₄ are each —(CH₂)ₙ—, wherein n is 2 or 3;
L₁, L₅, L₇, L₈, L₁₀, L₁₂ form an ester;
L₆, L₉, L₁₁, and L₁₃ form an amide; and
PDₓₓ and PD_{dd} are residues selected from the group consisting of 3,4-dihydroxyphenylethylamine, 3-methoxytyramine.

19. A bioadhesive construct, comprising:
a support suitable for tissue repair or reconstruction; and
a coating comprising a phenyl derivative (PD) functionalized polymer (PDp) of claim 1.

20. The bioadhesive construct of claim 20, further comprising an oxidant.

21. The bioadhesive construct of claim 21, wherein the oxidant is formulated with the coating.

22. The bioadhesive construct of claim 21, wherein the oxidant is applied to the coating.

23. The bioadhesive construct of claim 20, wherein the support is a film, mesh, a membrane, a nonwoven or a prosthetic.

24. A blend of a polymer and a compound of claim 1.

25. The blend of claim 24, wherein the polymer is present in a range of about 1 to about 50 percent by weight.

26. The blend of claim 25, wherein the polymer is present in a range of about 30 percent by weight.

27. A bioadhesive construct comprising:
a support suitable for tissue repair or reconstruction; and
a coating comprising the blend of claim 24.

28. The bioadhesive construct of claim 27, further comprising an oxidant.

29. The bioadhesive construct of claim 28, wherein the oxidant is formulated with the coating.

30. The bioadhesive construct of claim 28, wherein the oxidant is applied to the coating.

31. The bioadhesive construct of claim 27, wherein the support is a film, a mesh, a membrane, a nonwoven or a prosthetic.

32. A bioadhesive construct comprising:
a support suitable for tissue repair or reconstruction;
a first coating comprising a phenyl derivative (PD) functionalized polymer (PDp) of claim 1 and a polymer; and
a second coating coated onto the first coating, wherein the second coating comprises a phenyl derivative (PD) functionalized polymer (PDp) of claim 1.

33. A bioadhesive construct comprising:
a support suitable for tissue repair or reconstruction;
a first coating comprising a first phenyl derivative (PD) functionalized polymer (PDp) of claim 1 and a first polymer; and
a second coating coated onto the first coating, wherein the second coating comprises a second phenyl derivative (PD) functionalized polymer (PDp) of claim 1 and a second polymer, wherein the first and second polymer may be the same or different and wherein the first and second PDp can be the same or different.

34. A bioadhesive construct comprising:
a support suitable for tissue repair or reconstruction;
a first coating comprising a first phenyl derivative (PD) functionalized polymer (PDp) of claim 1; and
a second coating coated onto the first coating, wherein the second coating comprises a second phenyl derivative (PD) functionalized polymer (PDp) of claim 1, wherein the first and second PDp can be the same or different.
